SEQUENCE LISTING (43 sequences)

SEQ ID NO: 1 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 217 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNHFTQKSLSLSPGK

SEQ ID NO: 3 - protein, 15 aa, Homo sapiens
EPKSCDKTHTCPPCP

SEQ ID NO: 4 - protein, 10 aa, Homo sapiens
DKTHTCPPCP

SEQ ID NO: 5 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 6 - DNA, 575 bp, Artificial
  Synthetic polynucleotide
ctgcaggtgttcactcctgcgtcaacccccccaccgtgcagaacgcctacatcgtgtcccggcagatgagcaagtacccctccggtgagagggtgagataccagtgccgttccccctacgagatgttcggggacgaggaggtgatgtgcctcaacggtaactggaccgagcccccccagtgcaaggactccaccggtaagtgtggaccccccccccccatcgacaacggcgggatcacctccttcccgctgtccgtctacgccccggcctcctccgtcgagtatcagtgccagaacctgtaccagctcgagggcaacaagaggatcacctgccgtaacggtcagtggtccgagccccccaagtgcctccatccctgcgtgatcagccgtgagatcatggagaactacaacatcgccctgcggtggaccgccaagcagaagctctactccaggaccggcgagagcgtcgagtttgtgtgcaagcgtggttaccggctctcctccaggtcccataccctcaggaccacctgctgggacggcaagctcgagtaccccacctgtgccaagagggagctc

SEQ ID NO: 7 - DNA, 1329 bp, Artificial
  Synthetic polynucleotide
atggagtggagctggatcttcttgttcttgctcagcggcactgcaggtgttcactcctgcgtcaacccccccaccgtgcagaacgcctacatcgtgtcccggcagatgagcaagtacccctccggtgagagggtgagataccagtgccgttccccctacgagatgttcggggacgaggaggtgatgtgcctcaacggtaactggaccgagcccccccagtgcaaggactccaccggtaagtgtggaccccccccccccatcgacaacggcgggatcacctccttcccgctgtccgtctacgccccggcctcctccgtcgagtatcagtgccagaacctgtaccagctcgagggcaacaagaggatcacctgccgtaacggtcagtggtccgagccccccaagtgcctccatccctgcgtgatcagccgtgagatcatggagaactacaacatcgccctgcggtggaccgccaagcagaagctctactccaggaccggcgagagcgtcgagtttgtgtgcaagcgtggttaccggctctcctccaggtcccataccctcaggaccacctgctgggacggcaagctcgagtaccccacctgtgccaagagggccgctgccggtgggagctctgagcctaagtcatgcgataagactcatacttgtcctccttgtccggcgcctgaacttcttggaggaccttctgttttcctttttcctcctaagccaaaggacactcttatgattagtaggactcctgaagttacttgcgttgttgttgatgtttctcacgaagatcctgaagtgaagtttaactggtatgttgatggagttgaggtgcacaatgctaagactaagcctcgtgaggaacaatataattctacttacagggtagtgtctgtgttgacagtgcttcatcaagattggcttaacgggaaagaatataagtgcaaggtttctaataaggctctcccggcgcccattgaaaagaccatttctaaggctaagggacaacctagagaacctcaagtttacactcttcctccttctcgtgatgagttaaccaagaaccaggtttctcttacttgtttggtgaagggattctacccaagtgacattgctgtggagtgggaatctaatggacagcctgagaacaactataagactactcctcctgttcttgattctgacggatcattcttcttatacagcaagttgaccgttgataagtccagatggcaacaaggaaatgttttcagttgctctgttatgcatgaagcattacataaccactacactcaaaagtctctttctctttcgccggggaaataa

SEQ ID NO: 8 - protein, 442 aa, Artificial
  Synthetic polypeptide
MEWSWIFLFLLSGTAGVHSCVNPPTVQNAYIVSRQMSKYPSGERVRYQCRSPYEMFGDEEVMCLNGNWTEPPQCKDSTGKCGPPPPIDNGGITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRAAAGGSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1341 bp, artificial
  Synthetic polynucleotide
atggagtggagctggatcttcttgttcttgctcagcggcactgcaggtgttcactcctgcgtcaacccccccaccgtgcagaacgcctacatcgtgtcccggcagatgagcaagtacccctccggtgagagggtgagataccagtgccgttccccctacgagatgttcggggacgaggaggtgatgtgcctcaacggtaactggaccgagcccccccagtgcaaggactccaccggtaagtgtggaccccccccccccatcgacaacggcgggatcacctccttcccgctgtccgtctacgccccggcctcctccgtcgagtatcagtgccagaacctgtaccagctcgagggcaacaagaggatcacctgccgtaacggtcagtggtccgagccccccaagtgcctccatccctgcgtgatcagccgtgagatcatggagaactacaacatcgccctgcggtggaccgccaagcagaagctctactccaggaccggcgagagcgtcgagtttgtgtgcaagcgtggttaccggctctcctccaggtcccataccctcaggaccacctgctgggacggcaagctcgagtaccccacctgtgccaagaggggcggcggtggttctggtggtggcgggagctctgagcctaagtcatgcgataagactcatacttgtcctccttgtccggcgcctgaacttcttggaggaccttctgttttcctttttcctcctaagccaaaggacactcttatgattagtaggactcctgaagttacttgcgttgttgttgatgtttctcacgaagatcctgaagtgaagtttaactggtatgttgatggagttgaggtgcacaatgctaagactaagcctcgtgaggaacaatataattctacttacagggtagtgtctgtgttgacagtgcttcatcaagattggcttaacgggaaagaatataagtgcaaggtttctaataaggctctcccggcgcccattgaaaagaccatttctaaggctaagggacaacctagagaacctcaagtttacactcttcctccttctcgtgatgagttaaccaagaaccaggtttctcttacttgtttggtgaagggattctacccaagtgacattgctgtggagtgggaatctaatggacagcctgagaacaactataagactactcctcctgttcttgattctgacggatcattcttcttatacagcaagttgaccgttgataagtccagatggcaacaaggaaatgttttcagttgctctgttatgcatgaagcattacataaccactacactcaaaagtctctttctctttcgccggggaaataa

SEQ ID NO: 10 - protein, 446 aa, artificial
  Synthetic polypeptide
MEWSWIFLFLLSGTAGVHSCVNPPTVQNAYIVSRQMSKYPSGERVRYQCRSPYEMFGDEEVMCLNGNWTEPPQCKDSTGKCGPPPPIDNGGITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGGGSGGGGSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1356 bp, Artificial
  Synthetic polynucleotide
atggagtggagctggatcttcttgttcttgctcagcggcactgcaggtgttcactcctgcgtcaacccccccaccgtgcagaacgcctacatcgtgtcccggcagatgagcaagtacccctccggtgagagggtgagataccagtgccgttccccctacgagatgttcggggacgaggaggtgatgtgcctcaacggtaactggaccgagcccccccagtgcaaggactccaccggtaagtgtggaccccccccccccatcgacaacggcgggatcacctccttcccgctgtccgtctacgccccggcctcctccgtcgagtatcagtgccagaacctgtaccagctcgagggcaacaagaggatcacctgccgtaacggtcagtggtccgagccccccaagtgcctccatccctgcgtgatcagccgtgagatcatggagaactacaacatcgccctgcggtggaccgccaagcagaagctctactccaggaccggcgagagcgtcgagtttgtgtgcaagcgtggttaccggctctcctccaggtcccataccctcaggaccacctgctgggacggcaagctcgagtaccccacctgtgccaagaggggcggcggtggttctgggggtggcgggtctggcggcggggggagctctgagcctaagtcatgcgataagactcatacttgtcctccttgtccggcgcctgaacttcttggaggaccttctgttttcctttttcctcctaagccaaaggacactcttatgattagtaggactcctgaagttacttgcgttgttgttgatgtttctcacgaagatcctgaagtgaagtttaactggtatgttgatggagttgaggtgcacaatgctaagactaagcctcgtgaggaacaatataattctacttacagggtagtgtctgtgttgacagtgcttcatcaagattggcttaacgggaaagaatataagtgcaaggtttctaataaggctctcccggcgcccattgaaaagaccatttctaaggctaagggacaacctagagaacctcaagtttacactcttcctccttctcgtgatgagttaaccaagaaccaggtttctcttacttgtttggtgaagggattctacccaagtgacattgctgtggagtgggaatctaatggacagcctgagaacaactataagactactcctcctgttcttgattctgacggatcattcttcttatacagcaagttgaccgttgataagtccagatggcaacaaggaaatgttttcagttgctctgttatgcatgaagcattacataaccactacactcaaaagtctctttctctttcgccggggaaataa

SEQ ID NO: 12 - protein, 451 aa, Artificial
  Synthetic polypeptide
MEWSWIFLFLLSGTAGVHSCVNPPTVQNAYIVSRQMSKYPSGERVRYQCRSPYEMFGDEEVMCLNGNWTEPPQCKDSTGKCGPPPPIDNGGITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGGGSGGGGSGGGGSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1314 bp, Artificial
  Synthetic polynucleotide
atggagtggagctggatcttcttgttcttgctcagcggcactgcaggtgttcactcctgcgtcaacccccccaccgtgcagaacgcctacatcgtgtcccggcagatgagcaagtacccctccggtgagagggtgagataccagtgccgttccccctacgagatgttcggggacgaggaggtgatgtgcctcaacggtaactggaccgagcccccccagtgcaaggactccaccggtaagtgtggaccccccccccccatcgacaacggcgggatcacctccttcccgctgtccgtctacgccccggcctcctccgtcgagtatcagtgccagaacctgtaccagctcgagggcaacaagaggatcacctgccgtaacggtcagtggtccgagccccccaagtgcctccatccctgcgtgatcagccgtgagatcatggagaactacaacatcgccctgcggtggaccgccaagcagaagctctactccaggaccggcgagagcgtcgagtttgtgtgcaagcgtggttaccggctctcctccaggtcccataccctcaggaccacctgctgggacggcaagctcgagtaccccacctgtgccaagaggagctctgagcctaagtcatgcgataagactcatacttgtcctccttgtccggcgcctgaacttcttggaggaccttctgttttcctttttcctcctaagccaaaggacactcttatgattagtaggactcctgaagttacttgcgttgttgttgatgtttctcacgaagatcctgaagtgaagtttaactggtatgttgatggagttgaggtgcacaatgctaagactaagcctcgtgaggaacaatataattctacttacagggtagtgtctgtgttgacagtgcttcatcaagattggcttaacgggaaagaatataagtgcaaggtttctaataaggctctcccggcgcccattgaaaagaccatttctaaggctaagggacaacctagagaacctcaagtttacactcttcctccttctcgtgatgagttaaccaagaaccaggtttctcttacttgtttggtgaagggattctacccaagtgacattgctgtggagtgggaatctaatggacagcctgagaacaactataagactactcctcctgttcttgattctgacggatcattcttcttatacagcaagttgaccgttgataagtccagatggcaacaaggaaatgttttcagttgctctgttatgcatgaagcattacataaccactacactcaaaagtctctttctctttcgccggggaaataa

SEQ ID NO: 14 - protein, 437 aa, Artificial
  Synthetic polypeptide
MEWSWIFLFLLSGTAGVHSCVNPPTVQNAYIVSRQMSKYPSGERVRYQCRSPYEMFGDEEVMCLNGNWTEPPQCKDSTGKCGPPPPIDNGGITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1338 bp, Artificial
  Synthetic polynucleotide
atggagtggagctggatcttcttgttcttgctcagcggcactgcaggtgttcactccgataagactcatacttgtcctccttgtccggcgcctgaacttcttggaggaccttctgttttcctttttcctcctaagccaaaggacactcttatgattagtaggactcctgaagttacttgcgttgttgttgatgtttctcacgaagatcctgaagtgcagtttaagtggtatgttgatggagttgaggtgcacaatgctaagactaagcctcgtgaggaacaatataattctactttcagggtagtgtctgtgttgacagtgcttcatcaagattggcttaacgggaaagaatataagtgcaaggtttctaataaggctctcccggcgcccattgaaaagaccatttctaagactaagggacaacctagagaacctcaagtttacactcttcctccttctcgtgaggagatgaccaagaaccaggtttctcttacttgtttggtgaagggattctacccaagtgacattgctgtggagtgggaatcttctggacagcctgagaacaactataacactactcctcctatgcttgattctgacggatcattcttcttatacagcaagttgaccgttgataagtccagatggcaacaaggaaatattttcagttgctctgttatgcatgaagcattacataaccacttcactcaaaagtctctttctctttcgccggggaaagcggccgcgggcggcggtggttctggtggtggcgggagcacctcctgcgtcaacccccccaccgtgcagaacgcctacatcgtgtcccggcagatgagcaagtacccctccggtgagagggtgagataccagtgccgttccccctacgagatgttcggggacgaggaggtgatgtgcctcaacggtaactggaccgagcccccccagtgcaaggactccaccggtaagtgtggaccccccccccccatcgacaacggcgggatcacctccttcccgctgtccgtctacgccccggcctcctccgtcgagtatcagtgccagaacctgtaccagctcgagggcaacaagaggatcacctgccgtaacggtcagtggtccgagccccccaagtgcctccatccctgcgtgatcagccgtgagatcatggagaactacaacatcgccctgcggtggaccgccaagcagaagctctactccaggaccggcgagagcgtcgagtttgtgtgcaagcgtggttaccggctctcctccaggtcccataccctcaggaccacctgctgggacggcaagctcgagtaccccacctgtgccaagaggtaa

SEQ ID NO: 16 - protein, 465 aa, Artificial
  Synthetic polypeptide
MEWSWIFLFLLSGTAGVHSDKTHTCPPCPAPELLGGPSVFLFPPKPPCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNHFTQKSLSLSPGKAAAGGGGSGGGGSTSCVNPPTVQNAYIVSRQMSKYPSGERVRYQCRSPYEMFGDEEVMCLNGNWTEPPQCKDSTGKCGPPPPIDNGGITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 17 - DNA, 8885 bp, Artificial
  Synthetic polynucleotide
aattccgcccctagaaatatttgcgactcttctggcatgtaatatttcgttaaatatgaagtgctccatttttattaactttaaataattggttgtacgatcactttcttatcaagcgttactaaaatgcgtcaatctctttgttcttccatattcatatgtcaaaatctatcaaaattcttatatatctttttcgaatttgaagtgaaatttcgataatttaaaattaaatagaacatatcattatttaggtgtcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggcgcggggcgcgcccggtccaaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaattcacaacacaaatcagatttatagagagatttataaaaaaaaaaaaaacaatggagtggagctggatcttcttgttcttgctcagcggcactgcaggtgttcactccgataagactcatacttgtcctccttgtccggcgcctgaacttcttggaggaccttctgttttcctttttcctcctaagccaaaggacactcttatgattagtaggactcctgaagttacttgcgttgttgttgatgtttctcacgaagatcctgaagtgcagtttaagtggtatgttgatggagttgaggtgcacaatgctaagactaagcctcgtgaggaacaatataattctactttcagggtagtgtctgtgttgacagtgcttcatcaagattggcttaacgggaaagaatataagtgcaaggtttctaataaggctctcccggcgcccattgaaaagaccatttctaagactaagggacaacctagagaacctcaagtttacactcttcctccttctcgtgaggagatgaccaagaaccaggtttctcttacttgtttggtgaagggattctacccaagtgacattgctgtggagtgggaatcttctggacagcctgagaacaactataacactactcctcctatgcttgattctgacggatcattcttcttatacagcaagttgaccgttgataagtccagatggcaacaaggaaatattttcagttgctctgttatgcatgaagcattacataaccacttcactcaaaagtctctttctctttcgccggggaaagcggccgcgggcggcggtggttctggtggtggcgggagcacctcctgcgtcaacccccccaccgtgcagaacgcctacatcgtgtcccggcagatgagcaagtacccctccggtgagagggtgagataccagtgccgttccccctacgagatgttcggggacgaggaggtgatgtgcctcaacggtaactggaccgagcccccccagtgcaaggactccaccggtaagtgtggaccccccccccccatcgacaacggcgggatcacctccttcccgctgtccgtctacgccccggcctcctccgtcgagtatcagtgccagaacctgtaccagctcgagggcaacaagaggatcacctgccgtaacggtcagtggtccgagccccccaagtgcctccatccctgcgtgatcagccgtgagatcatggagaactacaacatcgccctgcggtggaccgccaagcagaagctctactccaggaccggcgagagcgtcgagtttgtgtgcaagcgtggttaccggctctcctccaggtcccataccctcaggaccacctgctgggacggcaagctcgagtaccccacctgtgccaagaggtaatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgaccgggcggccggccgcccctagaaatatttgcgactcttctggcatgtaatatttcgttaaatatgaagtgctccatttttattaactttaaataattggttgtacgatcactttcttatcaagcgttactaaaatgcgtcaatctctttgttcttccatattcatatgtcaaaatctatcaaaattcttatatatctttttcgaatttgaagtgaaatttcgataatttaaaattaaatagaacatatcattatttaggtgtcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggcgggtggagggggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgtacatcaccgacgagcaaggcaagaccgagcgcctttccgacgctcaccgggctggttgccctcgccgctgggctggcggccgtctatggccctgcaaacgcgccagaaacgccgtcgaagccgtgtgcgagacaccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgacaagctacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggactctagctagaggatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgatcgaatactaacgtctctaccagatatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgactctagctagagtccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagttccttcccgcttcagtgacaacgtcgagcacagctgcgcaaggaacgcccgttgtggccagccacgatagccgcgctgcctcgtcttggagttcattcagggcaccggacaggtcggtcttgacaaaaagaaccgggcgcccctgcgctgacagccggaacacggcggcatcagagcagccgattgtttgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctcagtggctccttcaacgttgcggttctgtcagttccaaacgtaaaacggcttgtcccgcgtcatcggcgggggtcataacgtgactcccttaattctccgctcatgatcgatatccattgaagagcaagct

SEQ ID NO: 18 - DNA, 37 bp, Artificial
  Synthetic polynucleotide
cacctattgacaatgggggcattacttcattcccgtt

SEQ ID NO: 19 - DNA, 29 bp, Artificial
  Synthetic polynucleotide
gaattcgtgtgtgaatccgcccacagtac

SEQ ID NO: 20 - DNA, 32 bp, Artificial
  Synthetic polynucleotide
gccgcggggggcgagcccaaatcttgtgacaa

SEQ ID NO: 21 - DNA, 35 bp, Artificial
  Synthetic polynucleotide
agcccaaatcttgtgacaaaactcacacatgccca

SEQ ID NO: 22 - DNA, 22 bp, Artificial
  Synthetic polynucleotide
cgggtaaatgagtgctagctgg

SEQ ID NO: 23 - protein, 62 aa, Artificial; identical to SEQ ID NO: 5
  Synthetic polypeptide

SEQ ID NO: 24 - DNA, 63 bp, Artificial
  Synthetic polynucleotide
gaaagcggccgcgggcggcggtggttctggtggtggcgggagcgactccaccggtaagtgtgg

SEQ ID NO: 25 - DNA, 30 bp, Artificial
  Synthetic polynucleotide
tttctctagattacctcttggcacaggtgg

SEQ ID NO: 26 - DNA, 63 bp, Artificial
  Synthetic polynucleotide
gaaagcggccgcgggcggcggtggttctggtggtggcgggagccatccctgcgtgatcagccg

SEQ ID NO: 27 - DNA, 30 bp, Artificial; identical to SEQ ID NO: 25
  Synthetic polynucleotide

SEQ ID NO: 28 - DNA, 45 bp, Artificial
  Synthetic polynucleotide
gagggttacgagctctcctcctcctcccataccctcaggaccacc

SEQ ID NO: 29 - DNA, 44 bp, Artificial
  Synthetic polynucleotide
ggaggaggagagctcgtaaccctccttgcacacaaactcgacgc

SEQ ID NO: 30 - DNA, 45 bp, Artificial
  Synthetic polynucleotide
ctcggttacaacctctcctcctcctcccataccctcaggaccacc

SEQ ID NO: 31 - DNA, 44 bp, Artificial
  Synthetic polynucleotide
ggaggaggagaggttgtaaccgagcttgcacacaaactcgacgc

SEQ ID NO: 32 - DNA, 42 bp, Artificial
  Synthetic polynucleotide
actctttacattaccagggagcctgaagttacttgcgttgtt

SEQ ID NO: 33 - DNA, 39 bp, Artificial
  Synthetic polynucleotide
aggctccctggtaatgtaaagagtgtcctttggcttagg

SEQ ID NO: 34 - DNA, 48 bp, Artificial
  Synthetic polynucleotide
tctgttcttcatgaagcattacattctcacttcactcaaaagtctctt

SEQ ID NO: 35 - DNA, 45 bp, Artificial
  Synthetic polynucleotide
gtgagaatgtaatgcttcatgaagaacagagcaactgaaaatatt

SEQ ID NO: 36 - protein, 217 aa, Artificial
  Synthetic polypeptide
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAMEWESSGQPENNYKTTPPVLDSDGSFFLYSKLTVDKSRWQQGNIFSCSVMHEALHNHYTQKSLSLSPGK

SEQ ID NO: 37 - DNA, 17 bp, Artificial
  Synthetic polynucleotide
ctgcaggtgttcactcc

SEQ ID NO: 38 - protein, 5 aa, Artificial
  Synthetic polypeptide
GGGGS

SEQ ID NO: 39 - protein, 10 aa, Artificial
  Synthetic polypeptide
GGGGSGGGGS

SEQ ID NO: 40 - protein, 15 aa, Artificial
  Synthetic polynucleotide
GGGGSGGGGSGGGGS

SEQ ID NO: 41 - protein, 6 aa, Artificial
  Synthetic polypeptide
GGGGSS

SEQ ID NO: 42 - protein, 11 aa, Artificial
  Synthetic polypeptide
GGGGSGGGGSS

SEQ ID NO: 43 - protein, 16 aa, Artificial
  Synthetic polypeptide
GGGGSGGGGSGGGGSS

(19) United States
(12) Patent Application Publication
(10) Pub. No.: US 2023/0357338 A1
(43) Pub. Date: Nov. 9, 2023

(54) FACTOR H FRAGMENT Fc FUSIONS WITH IMPROVED POTENCY AND MANUFACTURABILITY

(71) Applicant: Planet Biotechnology, Inc., Hayward, CA (US)

(72) Inventors: Keith WYCOFF, Hayward, CA (US); Y TRAN, Hayward, CA (US)

(73) Assignee: Planet Biotechnology, Inc., Hayward, CA (US)

(21) Appl. No.: 18/245,453

(22) PCT Filed: Sep. 15, 2021
(86) PCT No.: PCT/US2021/050533

§ 371 Date: Mar. 15, 2023

Related U.S. Application Data
(60) Provisional application No. 63/204,194, filed on Sep. 16, 2020.
(60) Provisional application No. 63/258,022, filed on Apr. 5, 2021.
(60) Provisional application No. 63/259,003, filed on Jun. 11, 2021.

(51) Int. Cl. C07K 14/47; A61P 31/04; A61K 38/17

(52) U.S. Cl. CPC C07K 14/4702; A61P 31/04; A61K 38/1725; C07K 2319/30

(57) ABSTRACT

The present disclosure relates generally to protein fusions of Factor H (FH) and Fc, where the fusions include variant linkers, additional N-terminal amino acids, and other variants of the linear structure and amino acid sequences that provide the enhanced microbicidal efficacy and/or manufacturability of the proteins. The present disclosure also provides compositions comprising these protein fusions or their encoding polynucleotide sequences, methods for their preparation, including recombinant production in plant hosts, and the use of these protein fusions for the reduction or eradication of pathogenic microbes in organisms, including prophylactic or therapeutic treatment of mammals, such as humans, for diseases caused by pathogenic microbes, including Lyme disease.

### CROSS REFERENCE TO RELATED APPLICATIONS

This application claims priority from U.S. provisional patent application 63/204,194, filed Sep. 16, 2020, U.S. provisional patent application 63/258,022 filed Apr. 5, 2021 and U.S. provisional patent application 63/259,003 filed Jun. 11, 2021, each of which is hereby incorporated by reference herein for all purposes.

### FIELD

The present disclosure relates to protein fusions of Factor H fragment and an Fc region capable of binding to pathogens, and methods for the use and manufacture of these proteins.

### REFERENCE TO SEQUENCE LISTING

The official copy of the Sequence Listing is submitted concurrently with the specification as an ASCII formatted text file via EFS-Web, with a file name of “16188-002WO1_SeqList_ST25.txt”, a creation date of Sep. 15, 2021, and a size of 54,192 bytes. The Sequence Listing filed herewith is part of the specification and is incorporated in its entirety by reference herein.

### BACKGROUND

Antimicrobial resistance is a major public health problem worldwide. Neisseria gonorrhoeae (Ng), the causative agent of the sexually transmitted infection gonorrhea, has become multidrug-resistant and has achieved “superbug” status. In addition, between 6% and 12% of women successfully treated for gonorrhea are re-infected within three months. Over the years, N. gonorrhoeae has become resistant to almost every antibiotic that has been used for treatment (Unemo et al. 2014, Unemo et al. 2019). The recent emergence of azithromycin-resistant isolates in several countries (Xue et al. 2015, Brunner et al. 2016, Liang et al. 2016, Katz et al. 2017) could render the first-line therapy for Ng, ceftriaxone plus azithromycin, recommended by the Centers for Disease Control and Prevention (see e.g., https://www.cdc.gov/std/treatment-guidelines/gonorrhea-adults.htm) ineffective in the near future. Novel therapeutics against Ng are urgently needed.

Complement (or “C′”) is a key arm of innate immune defenses. A mechanism used by several pathogens to escape C′ is to bind to a host's C′ inhibitor called Factor H (referred to herein as “FH”). FH is a complement control protein that includes 20 short consensus repeat (or “SCR”) domains organized in a head-to-tail manner as a single chain. Only the four N-terminal SCR domains (domains 1-4) possess C′ inhibiting activity; the remainder of the FH molecule is important for recognition of host cell surfaces. By binding to the surface of host cells of with SCR domains 1-4 intact, FH protects the surface of the host cells from damage caused by C′ activation.

Many pathogens have evolved the ability to bind FH domains 6-7 and/or 18-20 and thereby coopt the protective domains of Factor H so that the pathogen is protected by the same C′ inhibiting activity of FH domains 1-4. This strategy is used by a number of human pathogens including Neisseria gonorrhoeae (Ng), N. meningitidis, group A streptococci and non-typeable Haemophilus influenzae infections. Ng binds two FH regions: FH domains 6-7 and FH domains 18-20. In addition, the same strategy is used by tick-borne pathogens such as bacteria identified as the cause of Lyme disease, in particular Borrelia burgdorferi and other Borrelia species including Borrelia burgdorferi sensu lato (collectively referred to herein as the “Lyme borreliae”), B. burgdorferi sensu stricto (Bb) and B. afzelii (Ba), B. garinii (Bg), B. bavariensis (Bbav), and B. miyamotoi (Bm).

Tickborne pathogens (TBPs) have evolved immune-evasion strategies, both within the human host and in ticks' blood meal (Hart et al. 2018). The complement system is a critical component of innate immune defense that is central to controlling pathogen infections. Host cells are protected from complement attack by host complement regulatory proteins. TBPs use these same host proteins to escape from complement-mediated killing. For example, Lyme borreliae produce the outer surface proteins CspA, CspZ and OspE paralogs (hereafter OspE), whereas the relapsing fever borreliae Bm produces CbiA (Skare et al. 2020, Lin et al. 2020). Unlike OspA, which rapidly diminishes from the surface of Lyme borreliae after transmission from tick vector into the host, these proteins, which are present on these pathogens both within the tick vector and persist after tick bite and transmission into the human or animal host, recruit the soluble host complement inhibitor, FH, to the bacterial surface to inactivate complement, resulting in pathogen survival in host tissue and/or blood (Hart et al. 2018). CspA- and CspZ-mediated FH-binding activity facilitates spirochete transmission from ticks to host and dissemination in hosts, respectively, indicating the pivotal role of FH-mediated complement evasion in promoting bacterial survival in the infection cycle (Rottgerding et al. 2017, Kraiczy et al. 2013). Further, even non-Ixodes tick transmitted TBPs, such as Rickettsia sp. (Rocky Mountain spotted Fever) and Francisella tularensis (Tularemia), have been shown to escape complement attack by binding FH (Riley et al. 2012, Ben Nasr et al. 2008). These findings support FH-binding mediated complement evasion as a convergent mechanism for TBPs to evade host immune responses.

A recombinant fusion of a IgG Fc region and FH domains 18-20 has been identified with a point mutation at position 1119 of the FH domain 19, which abrogates binding to host cells. This point mutant has been designated herein as “FH”, and its fusion with an Fc region is designated herein as “FH*/Fc.” FH*/Fc has been found to bind to and promotes Complement (C′)-dependent killing of Ng but does not lyse human erythrocytes. Also previously described are fusions of FH* and Fc using a linker of at least 2 amino acids, but only linkers with specific Gly-Ala composition are disclosed. Topically administered FH*/Fc has been found to attenuate Ng infection in the mouse vaginal colonization model.

FH*/Fc has previously been made in mammalian cell culture and the interposition of linkers having at least two amino acids between the FH* and Fc sequences has been suggested as well. Two linker sequences fitting this description GAAGG and AAAGG have been previously disclosed (see e.g., Shaughnessy et al. 2016, and U.S. Pat. No. 10,975,131, each of which is hereby incorporated by reference herein). Furthermore, a general strategy for forming fusion proteins of an Fc region and another protein sequence so that the protein recognizes and binds to another molecule, that may for example be found on the surface of a pathogen, has been described in the art. In general, to generate a fusion protein, the sequences encoding the hinge region of an Ig (immunoglobulin) are retained and a region coding for a short (e.g., about 5 amino acid) linker is added between the pathogen recognition module coding region and the region coding for the Fc (N-terminal to the hinge). The main effector region of the Fc (i.e., the region that binds complement and protein A, and the single glycosylation site that is required to stabilize an Fc dimer—the effector functions are C-terminal to the hinge region) should be included. The previously suggested linkers comprise at least one alanine and/or glycine residue, and in addition to glycine residues remaining as part to the Fc region can include from 2 to 7 additional amino acid residues. Small, slightly hydrophilic amino acids such as glycine, alanine, serine, threonine, and methionine are preferred over charged, ring or aromatic amino acid residues. Specific examples of such linkers include the before-mentioned GAAGG and AAAGG.

IgG Fc fusions of FH domains 6-7 also have been previously disclosed; however, the fusions of FH 6-7-Fc are not described as having amino acids or particular combinations of amino acids as linker(s) interposed between the FH domains 6-7 and the Fc region (see e.g., Wong et al. 2016, and Shaughnessy et al. 2018).

The production of immunoadhesins, which are fusion proteins or glycoproteins comprised of an immunoglobulin Fc region and a ligand able to bind a target or a receptor to which another molecule usually binds, in planta is well known and previously described in detail in U.S. Pat. No. 7,591,378, which is hereby incorporated by reference herein. Expression of an FH*/Fc immunoadhesin in tobacco plants has been achieved with high yields (300-600 mg/kg biomass). The activity of the plant-produced FH*/Fc against gonococci was found to be similar to a CHO-cell produced FH*/Fc, both in vitro and in a mouse vaginal colonization model of gonorrhea.

### SUMMARY

The present disclosure relates generally to protein fusions of FH and Fc, with variant linker, additional N-terminal amino acids, and other variants of the linear structure and amino acid sequences that provide the enhanced microbicidal efficacy and/or manufacturability of the proteins. The present disclosure also provides compositions comprising these protein fusions or their encoding polynucleotide sequences, methods for their preparation, including recombinant production in plant hosts, and the use of these protein fusions for the reduction or eradication of pathogenic microbes in organisms, including prophylactic or therapeutic treatment of mammals, such as humans, for diseases caused by pathogenic microbes, including Lyme disease. This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure provides a fusion protein comprising an Fc and at least one Factor H (FH) short consensus repeat (SCR) domain capable of binding to a pathogen, wherein the Fc and FH SCR domains are fused by a linker consisting of glycine and serine residues; optionally, wherein the at least one FH SCR domain is selected from the group consisting of SCR 20, SCR 19-20, SCR 18-20, and SCR 6-7. In at least one embodiment, the fusion protein is selected from S2366, S2368, S2370, S2381, S2417, S2477, S2479, S2481, S2493, S2499, S2507, S2509, S2534, S2538, and S2635.

In at least one embodiment of the fusion protein of the present disclosure, the at least one FH SCR domain is domain 19 and has a point mutation at position 1119 which abrogates binding to host cells.

In at least one embodiment of the fusion protein of the present disclosure, the number of glycine residues exceeds the number of serine residues in the linker.

In at least one embodiment of the fusion protein of the present disclosure, the ratio of glycine residues to serine residues in the linker is 4 to 1. In at least one embodiment, the number of amino acid residues in the linker is selected from 5, 10, and 15. In at least one embodiment, the linker is selected from the group consisting of GGGGS, (GGGGS)₂ and (GGGGS)₃; optionally, wherein the linker comprises an amino acid sequence selected from SEQ ID NO: 38-43.

In at least one embodiment of the fusion protein of the present disclosure, the at least one FH SCR is at the N-terminus and the Fc is at the C-terminus of the fusion protein.

In at least one embodiment of the fusion protein of the present disclosure, the at least one FH SCR is at the C-terminus and said the Fc is at the N-terminus of the fusion protein.

In at least one embodiment of the fusion protein of the present disclosure, the Fc comprises Fc of human IgG1 or IgG3. In at least one embodiment, the fusion protein further comprises the hinge region of IgG1; optionally, wherein the hinge region comprises an amino acid sequence selected from SEQ ID NO: 3, and 4. In at least one embodiment, the fusion protein further comprises the hinge region of IgG3; optionally, wherein the hinge region comprises an amino acid sequence selected from SEQ ID NO: 5, and 23.

In at least one embodiment of the fusion protein of the present disclosure, the at least one FH SCR domain is domain 19 and has a point mutation at position 1119 which abrogates binding to host cells and further comprises additional N-terminal amino acids attached to FH*, wherein the additional N-terminal amino acids are selected from the group consisting of: TS (threonine, and serine); DTS (aspartic acid, threonine, and serine); and RDTS (arginine, aspartic acid, threonine, and serine). In at least one embodiment, the amino acid sequence of the fusion protein has the linear structure: N-terminus-[additional N-terminal amino acids]-FH*-linker-Fc-C-terminus. In at least one embodiment, the amino acid sequence of the fusion protein has the linear structure: N-terminus-Fc-linker-[additional N-terminal amino acids]FH*-C-terminus. In at least one embodiment, the fusion protein has the linear structure selected from: (i) N-terminus-Fc-linker-TS-FH*-C-terminus; (ii) N-terminus-Fc-linker-DTS-FH*-C-terminus; and (iii) N-terminus-Fc-linker-RDTS-FH*-C-terminus.

In at least one embodiment of the fusion protein of the present disclosure, the Fc comprises Fc of human IgG1 or IgG3 and the at least one FH SCR domain capable of binding to a pathogen is SCR 6-7.

In at least one embodiment of the fusion protein of the present disclosure, the Fc comprises Fc of human IgG1 and further comprises the hinge region of IgG1.

In at least one embodiment of the fusion protein of the present disclosure, the Fc comprises Fc of human IgG3 and further comprises the hinge region of IgG3.

In at least one embodiment of the fusion protein of the present disclosure, the at least one FH SCR domain is SCR 20, and the SCR 20 domain comprises amino acid modifications selected from the group consisting of: R1203E, R1206E, and R1210S or R1203UR1206N/R1210S.

In at least one embodiment of the fusion protein of the present disclosure, the Fc is IgG3 Fc and comprises amino acid modifications thereof selected from the group consisting of: M252Y/S254T/T256E or M428L/N434S.

In at least one embodiment of the fusion protein of the present disclosure, the Fc comprises the amino acid sequence of IgHg3*17; optionally, wherein the amino acid sequence comprises SEQ ID NO: 36.

In at least one embodiment, the present disclosure provides an FH 6-7/Fc and FH*/Fc fusion protein with enhanced microbicidal efficacy or enhanced opsinophagocytotic efficacy.

In another aspect, the present provides a polynucleotide or an expression vector encoding a fusion protein of the present disclosure. In at least one embodiment, the expression vector is suitable for expressing the polynucleotide in a mammalian host cell or a mammalian tissue; optionally, wherein the mammalian cell or tissue comprises a CHO cell. In at least one embodiment, the expression vector is suitable for expressing the polynucleotide in a plant cell or plant tissue; optionally, wherein plant cell or tissue is from N. benthamiana.

In another aspect, the present disclosure provides a method for reducing the duration and/or burden of colonization of a microbe in a mammalian host, the method comprising providing to the mammalian host a fusion protein of the present disclosure in an amount effective to reduce the duration and/or burden of colonization. In at least one embodiment of the method, the microbes are selected from the group consisting of Neisseria gonorrhoeae (Ng), N. meningitidis, group A streptococci, methicillin resistant Staphylococcus aureus non-typeable Haemophilus influenzae, Borrelia burgdorferi sensu lato (collectively referred to as the Lyme borreliae), B. burgdorferi sensu stricto (Bb) and B. afzelii (Ba), B. garinii (Bg), B. bavariensis (Bbav), Borrelia miyamotoi (Bm), Rickettsia sp., and Francisella tularensis.

In at least one embodiment, the present disclosure provides a method for reducing a population of pathogenic microbes in an organism, the method comprising treating the organism with an effective amount a fusion protein of the present disclosure. In at least one embodiment of the method, the microbes are selected from the group consisting of Neisseria gonorrhoeae (Ng), N. meningitidis, group A streptococci, methicillin resistant Staphylococcus aureus non-typeable Haemophilus influenzae, Borrelia burgdorferi sensu lato (collectively referred to as the Lyme borreliae), B. burgdorferi sensu stricto (Bb) and B. afzelii (Ba), B. garinii (Bg), B. bavariensis (Bbav), Borrelia miyamotoi (Bm), Rickettsia sp., and Francisella tularensis.

In at least one embodiment, the present disclosure provides a method for preventing and/or treating a microbe infection in a subject, the method comprising administering to the subject an effective amount a fusion protein of the present disclosure. In at least one embodiment of the method, the microbes are selected from the group consisting of Neisseria gonorrhoeae (Ng), N. meningitidis, group A streptococci, methicillin resistant Staphylococcus aureus non-typeable Haemophilus influenzae, Borrelia burgdorferi sensu lato (collectively referred to as the Lyme borreliae), B. burgdorferi sensu stricto (Bb) and B. afzelii (Ba), B. garinii (Bg), B. bavariensis (Bbav), Borrelia miyamotoi (Bm), Rickettsia sp., and Francisella tularensis.

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1A, FIG. 1B, FIG. 1C, and FIG. 1D depict plots of results obtained using FH*/Fc fusion proteins of the present disclosure measured as described in the Examples. FIG. 1A depicts plots of results of binding affinity to N. gonorrhoeae H041 as measured by median fluorescence for four variant FH*/Fc fusion proteins of the present disclosure as produced in tobacco plants as compared to a FH*/FC fusion protein made in CHO cells. FIG. 1B shows serum microbicidal activity assessed in bactericidal assays using N. gonorrhoeae H041 bacteria for four variant FH*/Fc fusion proteins of the present disclosure produced in tobacco plants as compared to a FH*/FC produced in CHO cells. FIG. 1C shows opsonophagocytic killing of gonococci with freshly isolated human polymorphonuclear leukocytes (PMNs), for four variant FH*/Fc fusion proteins of the present disclosure, wherein the variants have: (i) no linker; (ii) AAAGG linker; (iii) (GGGGS)₂ linker; or (GGGGS)₃ linker. FIG. 1D shows the result of bactericidal testing of FH*/Fc fusion proteins of the present disclosure comprising a (GGGGS)₃ linker against five additional gonococcal strains.

FIG. 2A and FIG. 2B depict plots of results showing the effect of FH*/Fc fusion protein with the (GGGGS)₃ linker (S2370) in attenuating both the duration and the burden of gonococcal vaginal colonization model using FH/C4BP transgenic mice. The efficacy of the FH*/Fc fusion protein, S2370 against N. gonorrhoeae H041 was determine in human FH/C4BP transgenic mice. Premarin®-treated 6-8 week-old human FH/C4BP transgenic mice (n=6/group) were infected with either 10⁶ CFU (FIG. 2A) or 10⁷ CFU (FIG. 2B) of N. gonorrhoeae strain H041. Mice were treated daily (starting 2 h before infection) intravaginally either with PBS (vehicle control) or with 1 μg or 10 μg of the S2370 FH*/Fc fusion. The left panels of FIG. 2A and FIG. 2B depict Kaplan Meier curves showing time to clearance, analyzed the Mantel-Cox (log-rank) test. Significance was set at 0.017 (Bonferroni's correction for comparisons across three groups). The middle panels of FIG. 2A and FIG. 2B show log₁₀ CFU versus time. X-axis, day; Y-axis, log₁₀ CFU. Comparisons of the CFU over time between each treatment group and the respective saline control was made by two-way ANOVA and Dunnett's multiple comparison test. *, P<0.05; **, P<0.01; ***, P<0.001; ****, P<0.0001. The right panels of FIG. 2A and FIG. 2B show the bacterial burdens consolidated over time (Area Under the Curve [log₁₀ CFU] analysis). The three groups were compared by one-way ANOVA using the non-parametric Kruskal-Wallis equality of populations rank test. The X2 with ties were 12.12 (P=0.0002) and 11.94 (P=0.0002) for the graphs FIG. 2A and FIG. 2B, respectively. Pairwise AUC comparisons across groups were made with Dunn's multiple comparison test.

FIG. 3 depict plots showing the efficacy of a FH*/Fc fusion protein having the (GGGGS)₃ linker (S2370) against N. gonorrhoeae FA1090 in human FH/C4BP transgenic mice. Premarin®-treated 6 week-old human FH/C4BP transgenic mice (n=8/group) were infected with 4×10⁷ CFU N. gonorrhoeae strain FA1090. Mice were treated daily (starting 2 h before infection) intravaginally either with PBS (vehicle control) or with 10 μg of FH*/Fc molecule S2370. Left panel FIG. 3 depicts Kaplan Meier curves showing time to clearance, analyzed the Mantel-Cox (log-rank) test. The middle panel of FIG. 3 depicts plots of log₁₀ CFU versus time. X-axis, day; Y-axis, log₁₀ CFU. Comparisons of the CFU over time between each treatment group and the respective saline control was made by two-way ANOVA and Dunnett's multiple comparison test. ***, P<0.001; ****, P<0.0001. The right panel of FIG. 3 shows plotted bacterial burdens consolidated over time (Area Under the Curve [log₁₀ CFU] analysis). Comparisons were made by Mann-Whitney's non-parametric test.

FIG. 4A, FIG. 4B, and FIG. 4C depict results of the characterization of a FH*/FC fusion protein, s2477, which has the N-terminal cysteine of FH* capped with the two additional amino acids, TS, and yields fewer degradation products after purification while maintaining effective microbicidal activity, as compared to the S2370 fusion protein. FIG. 4A: S2477 has fewer degradation products compared to S2370 as shown by the Western blot image of purified S2377 (lane 1) and S2360 (lane 2) using anti-human IgG alkaline phosphatase as the detection reagent. Note that irrelevant lanes between lanes 1 and 2 have been excluded. MW, molecular weight. FIG. 4B: the S2477 (TS-FH*-(G₄S)₂/Fc) and the S2370 (FH*-(G₄S)₃/Fc) fusion proteins (concentrations indicated on the X-axis) were incubated with sialylated Ng strain H041 and complement and survival at 30 min (relative to 0 min) was measured in a bactericidal assay. Fig. C: Complement-dependent bactericidal efficacy of S2477 against N. gonorrhoeae strain NJ-60. Negative controls included bacteria incubated with complement alone (open bar on left) and bacteria incubated with 8 μg/ml S2493 (TS-FH*-(G₄S)₂/Fc-D270A/K322A (complement-inactive Fc mutations); hatched bar on right)

FIG. 5 shows the map of FH*/Fc plasmids in pTRAkc. The open reading frame is between the CHS enhancer and the 35S polyadenylation signal (pA35S)

FIG. 6 shows the microbicidal activity of hFc3(GGGGS)₂-(TS)FH* against a panel of 50 strains of N. gonorrhoeae.

FIG. 7A and FIG. 7B depict plots of results showing the microbicidal activity of different position variants of two variant fusion proteins of the present disclosure against strain H041 of N. gonorrhoeae. FIG. 7A shows plots for the variant FH(D119G)/Fc fusion proteins, S2477, S2509, and S2534. FIG. 7B shows plots for the variant FH(D119G)/Fc fusion proteins, S2477, S2499, and S2509.

FIG. 8 depicts a schematic illustration of in vitro and in vivo complement-mediated pathogen killing by an FH6-7/Fc fusion protein. (i) the FH portion of the fusion protein binds to the pathogen cell surface and prevents binding by endogenous FH and displaces bound FH from the pathogen surface; (ii) binding to the pathogen enables the Fc portion of the fusion protein to engage the C1 complex which deposits C4b and C3b on the pathogen resulting in a membrane attack complex, (C5b-9) insertion in the membrane; (iii) the Fc and iC3b (generated by cleavage of C3b) engage FcγR and CR3, thereby enhancing phagocytosis.

FIG. 9 depicts plots of results showing the complement mediated killing of two strains of B. burgdorferi and B. afzelii facilitated by three different FH/Fc fusion constructs: FH 6-7/Fc, FH*/Fc, and a FH 6-7/Fc mutant containing two Fc mutations (D270A/K322A) that eliminate C1q binding. The FH/Fc fusion constructs, SCR(6-7)/Fc1 (wt), SCR(6-7)/Fc1 (mt), and SCR(18-20)/Fc1 (FH*/Fc1), or BSA were mixed at indicated dilutions with 40% human serum (complement source) and B. burgdorferi strains B31-5A4 and 297 and B. afzelii strain VS461. Surviving spirochetes were quantified using dark-field microscopy after 24 hr. Percent survival was derived from the proportion of FH/Fc-treated to untreated spirochetes. Data shown are the mean±SD of percent survival derived from three microscope fields for each sample.

FIG. 10 depicts results showing that B. burgdorferi is eliminated in nymphs feeding on mice treated with the FH 6-7/Fc3 fusion protein and significantly reduced on mice treated with FH*/Fc3 at the dose of 20 mg/kg. The Dotted line is the limit of detection (10 bacteria per tick). Shown is the geometric mean±geometric SD. (*) indicates the significant difference at p<0.05.

FIG. 11 depicts results of a study showing that pre-treatment of mice with FH 6-7/Fc3 at the dose of 20 mg/kg prevents mice from contracting B burgdorferi infection from ticks infected with and carrying the bacteria. Dotted line is the limit of detection (10 bacteria per 100 ng total DNA). Shown is the geometric mean±geometric SD of bacterial burdens.

FIG. 12 depicts results of a preliminary pharmacokinetic study of FH/Fc fusion protein in BALB/c mice (3 per treatment) when administered FH 6-7/Fc1 or FH*/Fc1 at 5 mg/kg intravenously. Blood was collected for serum at 1, 8, 24 and 48 hr. Proteins were quantified by ELISA.

FIG. 13 depicts plots of results of assays of Complement Activated PMN killing of methicillin resistant Staphylococcus aureus mediated by 5% normal human serum and the fusion proteins: human FH* fused to the N-terminus of human IgG1 Fc (“FH-Fc”); human FH* fused to the N-terminus of human IgG1 Fc with two Fc mutations (D270A/K322A) (“V1”), which lacks the ability to activate complement; and human FH* fused to the N-terminus of human IgG3 Fc (“V2”).

FIG. 14 depicts plots of results of assays of Complement Activated PMN killing of methicillin resistant Staphylococcus by aureus mediated by 10% normal human serum and the fusion proteins: human FH* fused to the N-terminus of human IgG1 Fc (“FH-Fc”); human FH* fused to the C-terminus of human IgG1 Fc (“V2R”); and human FH* fused to the C-terminus of human IgG1 Fc with the addition of an EFT modification in the Fc region (“V5”), that has been shown to improve binding to C1q.

FIG. 15 depicts plots of results of assays of the influence of pH on the ability of FH*/Fc fusion protein, S2534, and the FH 6,7-Fc fusion protein, S2535 to bind to Ng H401.

FIG. 16 shows polynucleotide sequence encoding the fusion protein variant S2366 (SEQ ID NO: 7) as cloned in plasmid construct p1338.

FIG. 17 shows polynucleotide sequence encoding the fusion protein variant S2368 (SEQ ID NO: 9) as cloned in the plasmid construct p1339.

FIG. 18 shows polynucleotide sequence encoding the fusion protein variant S2370 (SEQ ID NO: 11) as cloned in the plasmid construct p1340.

FIG. 19 shows polynucleotide sequence encoding the fusion protein variant S2381 (SEQ ID NO: 13) as cloned in the plasmid construct p1346.

FIG. 20 summarizes the organization of the plasmid p1425 construct.

FIG. 21 shows the complete polynucleotide sequence of plasmid p1425 with associated features and encoded amino acid sequences, including the PTRAk sequence and all inserts in the open reading frame.

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of these limits, ranges excluding (i) either or (ii) both of those included limits are also included in the invention. For example, “1 to 50,” includes “2 to 25,” “5 to 20,” “25 to 50,” “1 to 10,” etc.

Generally, the nomenclature used herein and the techniques and procedures described herein include those that are well understood and commonly employed by those of ordinary skill in the art, such as the common techniques and methodologies described in e.g., Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00-130, (1987-2020), published by Wiley & Sons, Inc. in the Wiley Online Library (hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

The present disclosure provides variant FH*/Fc protein fusions that include structural features that result in increased yields of the intact, functional protein when produced in a recombinant host, and/or enhanced potency of the FH*/Fc when used for the control of microbial pathogens. The increased yield in production of the protein fusions also provides enhanced processibility for larger scale manufacture when the proteins are concentrated and purified from the recombinant host.

Factor H is a complement control protein that includes 20 short consensus repeat (or “SCR”) domains organized in a head-to-tail manner as a single chain. Only the four N-terminal SCR domains (domains 1-4) possess C′ inhibiting activity; the remainder of the FH molecule is important for recognition of host cell surfaces. By binding to the surface of host cells of with SCR domains 1-4 intact, FH protects the surface of the host cells from damage caused by C′ activation. As used herein, “FH domains” or “FH” or “SCR” followed by a number refers to one or more of the short consensus repeat (SCR) domains of Factor H. Thus, “FH domains 1-4” refers to SCR domains 1,2,3, and 4 at the N-terminal end of Factor H. “FH 6,7” refers to SCR domains 6 and 7 of Factor H, “FH 18-20” refers to SCR domains 18,19, and 20 of Factor H. “FH 19-20” refers to the SCR domains 19 and 20 of Factor H. “FH 20” refers to the SCR domain 20 of Factor H. “FH*” refers to FH with a point mutation at position 1119 of FH domain 19. This point mutation abrogates binding to host cells. FH*/Fc fusions have been found to bind to and promote C′-dependent killing of microorganisms, such as Ng, but with lysing human erythrocytes.

FH*/Fc fusion proteins have previously been produced in mammalian cell culture with yields that are low, and, as a result, it has been difficult to produce significant amounts of the recombinant fusions for clinical development. As shown in the present disclosure, improvements in yield of the intact FH*/Fc fusion protein can be obtained by producing the fusion molecule in plants. Additionally, it is a surprising discovery of the present disclosure that interposition of certain flexible linkers between FH* and Fc portions of the fusion molecule can increase yield of intact protein, which is a measure of increased processibility. Furthermore, these variant linker structures result in significantly enhanced microbicidal potency of the protein fusions. In at least one embodiment, the present disclosure provides plant-made variant FH*/Fc fusion proteins incorporating the flexible linkers, (GGGGS)₂ or (GGGGS)₃, which are provide functionally superior molecules when compared to known FH*/Fc fusion proteins that have alanine-containing linkers. The superior function of the FH*/Fc fusions having these flexible linker structures that include on glycine and serine residues is exhibited whether produced in mammalian cells or in plant cells, and when evaluated both in vitro and in a mouse vaginal infection prophylactic model of N. gonorrhea.

Accordingly, in at least one embodiment the present disclosure provides fusions of FH 6-7 or FH* with Fc and a flexible linker of at least two different amino acids, wherein the linker composition lacks alanine residues, between the FH 6-7 or FH* sequence and the Fc sequence. As used herein, the term “wherein the linker composition lacks alanine residues” refers to the contiguous linker sequence, and excludes amino acids that may flank the contiguous linker sequence. Preferably, the linker composition introduced between FH 6-7 or FH* and Fc that lacks alanine residues has a composition consisting of glycine (G) and serine (S). In a preferred embodiment the number of glycine residues exceeds the number of serines in the linker. Also preferred are fusions of FH 6-7 or FH* and Fc wherein the ratio of glycines to serine in the linker is 4 to 1, such as for example GGGGS. More preferred are fusions of FH 6-7 or FH* and Fc wherein the linker is between 5 and 15 amino acids in length and consists entirely of glycine and serine residues. Most preferred are fusions of FH 6-7 or FH* and Fc wherein the linker between FH 6-7 or FH* and Fc has the composition GGGGS, (GGGGS)₂ or (GGGGS)₃. These linkers also may be abbreviated in the following description as G₄S, (G₄S)₂ and (G₄S)₃, respectively. The foregoing FH* may be in extended form such that the N-terminal cysteine of FH* is capped, for example with an additional N-terminal amino acids (TS) as described above, and elsewhere herein.

Also as noted elsewhere herein, the variant structural features of FH*/Fc fusions provided herein act to enhance the manufacturability of recombinant production of these protein. After the FH*/Fc fusion protein is produced recombinantly in a plant, the plant biomass must be harvested, extracted, concentrated, and purified. Typically, the concentration, purification, and sterile filtration of FH*/Fc fusion proteins results in dramatic losses of protein; close to 50% versus the ˜20% loss seen with other plant-produced Fc fusions (Wycoff et al. 2011, Wycoff et al. 2015). A distinctive feature of FH*/Fc fusion proteins of the present disclosure that the presence of an N-terminal cysteine (amino acid 1048 of human FH (GenBank: AAI42700.1) (Shaughnessy et al. 2016). Proteins with N-terminal cysteines can undergo native chemical ligation, whereby the cysteine reacts with free thioester groups (Dawson et al. 1994, Gentle et al. 2004), which may lead to significant protein loss during concentration. It is a surprising advantageous discovery of the present disclosure that when a FH*/Fc fusion protein sequence is extended with as few as two additional amino acids that are N-terminal to the N-terminal cysteine residue, the level protein loss during processing is greatly reduced. Accordingly, in at least one embodiment the present disclosure provides an extended form of a FH*/Fc fusion protein that includes the two additional amino acids (TS) that are normally present N-terminal to the cysteine in the native FH protein sequence. In another embodiment of the disclosure, this extended form of the FH*/Fc can include at least three amino acid residues additional to the N-terminal cysteine, for example, in one preferred embodiment, the three amino acids DTS may be included in the FH*/Fc. In another embodiment of the invention the extended form provides four amino acid residues additional to the N-terminal cysteine. In one preferred four amino acid extended form, amino acids RDTS may be provided. Providing FH* in any of the described extended forms, substantially reduces the previously noted loss of protein during concentration and downstream purification of FH*/Fc.

The present disclosure also provides the unexpected and surprising discovery that reversing the order of the elements of the FH*/Fc fusion proteins can increase the potency of one fusion protein compared to the potency of a fusion protein having the same elements but the reverse order. Accordingly, in an additional aspect of the fusion proteins of FH* and Fc of the present disclosure are those fusion in which the position of FH* and the Fc have been reversed. Whereas the foregoing fusions are generally produced as proteins with an amino terminal FH*-linker-Fc carboxy terminal structure, it was found that reversing the order of the Fc and FH* in the fusion protein sequence (Fc-linker-FH*) led to a marked and measurable increase in potency of the fusion protein in killing the target bacteria, in this case N. gonorrhea. Thus, for example a fusion protein having the structure amino-terminal hFc1(GGGS)₂-(TS)-FH* has greater potency than the fusion protein having the structure amino-terminal (TS)-FH*-(GGGGS)₂-hFc1 and amino-terminal hFc3(GGGS)₂-(TS)-FH*, has greater potency than the fusion protein having the structure amino-terminal (TS)FH*-(GGGGS)₂-hFc3.

Also disclosed is a method to measurably improve the microbiocidal efficacy of FH 6-7/Fc and FH*/Fc fusions by providing FH 6-7/Fc or FH*/Fc having a flexible linker of at least two amino acids between the FH 6-7 or FH* sequence and the Fc sequence, wherein the linker composition lacks alanine residues and contacting microbes with said provided FH 6-7-linker-Fc or FH*-linker-Fc. Preferably the method to measurably improve the microbiocidal efficacy of FH/6-7 or FH*/Fc fusions by contacting microbes therewith, provides FH 6-7/Fc or FH*/Fc wherein the linker introduced between FH 6-7 or FH* and Fc that lacks alanine residues has a composition consisting of glycine (G) and serine (S). Also preferred is the method to measurably improve the microbiocidal efficacy of FH*/Fc fusions by contacting bacteria therewith by providing fusions of FH 6-7 or FH* and Fc wherein the linker has the composition GGGGS. More preferred is the method to measurably improve the microbiocidal efficacy of FH 6-7Fc or FH* Fc fusions by contacting microbes therewith by providing fusions of FH 6-7 or FH*and Fc where in the linker is 5 to 15 amino acids in length and consists entirely of Glycine and Serine. Most preferred is the method to measurably improve the microbiocidal efficacy of FH 6-7 or FH* Fc fusions by contacting bacteria therewith by providing fusions of FH 6-7 or FH* and Fc wherein the linker between FH 6-7 or FH* and Fc has the composition GGGGs, (GGGGS)₂ or (GGGGS)₃.

The term “microbiocidal” as used herein and as commonly understood means the ability to kill microorganisms including bacteria, viruses, protozoans, and fungi. Among the microorganisms that may be subjected to the improved microbiocidal activity of the FH6-7 linker Fc fusions and FH*-linker-/Fc fusions of the invention are Neisseria gonorrhoeae (Ng), N. meningitidis, group A streptococci, methicillin resistant Staphylococcus aureus, non-typeable Haemophilus influenzae, Borrelia burgdorferi sensu lato (collectively referred to as the Lyme borreliae), B. burgdorferi sensu stricto (Bb) and B. afzelii (Ba), B. garinii (Bg), B. bavariensis (Bbav) and Borrelia miyamotoi (Bm), Rickettsia sp., which causes Rocky Mountain spotted Fever, and Francisella tularensis, which causes Tularemia.

As used herein, the term “PMN” refers to granulocytes, which are a category of white blood cells in the innate immune system characterized by the presence of granules in their cytoplasm. They are also called polymorphonuclear leukocytes (PMN, PML, or PMNL) because of the varying shapes of the nucleus, which is usually lobed into three segments.

In addition, disclosed herein is a method to measurably improve the (PMN)-mediated opsonophagocytosis efficacy of FH 6-7/Fc or FH*/Fc fusions against a microorganism by providing FH 6-7/Fc or FH*/Fc having a flexible linker of at least two amino acids between the FH 6-7 or FH* sequence and Fc sequence, wherein the linker composition lacks alanine residues and contacting the microorganism with said provided FH 6-7-linker-Fc or FH*-linker-Fc. Preferably the method to measurably improve the PMN-mediated opsonophagocytosis efficacy of FH 6-7/Fc or FH*/Fc fusions against a microorganism provides FH/6-7 or FH*/Fc wherein the linker introduced between FH 6-7 or FH* and Fc that lacks alanine residues has a composition consisting of glycine (G) and serine (S). Also preferred is the method to measurably improve the PMN-mediated opsonophagocytosis efficacy of FH 6-7/Fc or FH*/Fc fusions against a microorganism by providing fusions of FH 6-7 or FH* and Fc wherein the linker has the composition GGGGS. More preferred is the method to measurably improve the PMN-mediated opsonophagocytosis efficacy of FH6-7Fc or FH*/Fc fusions against a microorganism such as bacteria by providing fusions of FH 6-7 or FH*and Fc where in the linker is 5 to 15 amino acids in length and consists entirely of glycine and serine. Most preferred is the method to measurably improve PMN-mediated opsonophagocytosis efficacy of FH 6-7/Fc or FH*/Fc fusions against a microorganism, by providing fusions of FH 6-7 or FH* and Fc, wherein the linker between FH 6-7 or FH* and Fc has the composition GGGGS, (GGGGS)₂ or (GGGGS)₃. Among the microorganisms that may demonstrate the improved opsonophagocytosis activity of the FH6-7-linker-/Fc fusion proteins and FW-linker-Fc fusion proteins of the invention are Neisseria gonorrhoeae (Ng), N. meningitidis, group A streptococci, methicillin resistant Staphylococcus aureus non-typeable Haemophilus influenzae, Borrelia burgdorferi sensu lato (collectively referred to as the Lyme borreliae), B. burgdorferi sensu stricto (Bb) and B. afzelii (Ba), B. garinii (Bg), B. bavariensis (Bbav) and Borrelia miyamotoi (Bm), Rickettsia sp., which causes Rocky Mountain spotted Fever, and Francisella tularensis, which causes Tularemia.

The present disclosure also provides a method to reduce the duration and/or burden of colonization of particular bacterial microorganisms that are either not drug resistant or are resistant to one or more antibiotic drugs, including but not limited to the above-indicated bacterial strains, including gonococcal strains, by providing to a mammalian host a FH6-7-linker-Fc or FH*-linker-Fc in an amount effective to reduce the duration and/or burden of colonization. An “effective amount” refers to the amount of an active ingredient (e.g., an FH*/Fc fusion protein) to achieve a desired microbiocidal result, e.g., to reduce or eradicate microorganisms in a sample or subject.

The present disclosure also provides a method for reducing a population of pathogenic microbes in an organism, the method comprising treating the organism with an effective amount of a FH6-7-linker-Fc or FH*-linker-Fc fusion protein.

It is contemplated that a composition or formulation comprising a fusion protein of the present disclosure can be used for any methods or uses, such as in therapeutic methods that utilize theft ability to reduce the population of pathogenic microbes in a subject, and thereby treat microbial infections and pathogen-associated symptoms and disease states in the subject. Accordingly, in at least one embodiment, the present disclosure also provides a method for treating an infection of microbes in a subject, the method comprising administering to the subject a therapeutically effective amount of a FH6-7-linker-Fc or FH*-linker-Fc fusion protein.

“Treatment,” “treat” or “treating” refers to clinical intervention in an attempt to alter the natural course of a disorder, infection, or pathology in an individual being treated, and can be performed either for prophylaxis or during the course of clinical pathology. Desired results of treatment can include, but are not limited to, preventing occurrence or recurrence of the disorder, infection, alleviation of symptoms, diminishment of any direct or indirect pathological consequences of the disorder or infection. For example, treatment can include administration of a therapeutically effective amount of pharmaceutical formulation comprising an FH*/Fc fusion protein to a subject to reduce or eradicate a microbial infection in the subject.

A “therapeutically effective amount” refers to the amount of an active ingredient (e.g., a fusion protein) to achieve a desired therapeutic or prophylactic result, e.g., to treat or prevent an Ng infection in a subject. In the case of a microbial infection or associated condition, the therapeutically effective amount of the therapeutic agent is an amount that reduces, prevents, inhibits, and/or relieves to some extent one or more of the symptoms associated with the infection or condition.

A “pharmaceutical formulation” refers to a preparation in a form that allows the biological activity of the active ingredient(s) to be effective, and which contain no additional components which are toxic to the subjects to which the formulation is administered. A pharmaceutical formulation may include one or more active agents. For example, a pharmaceutical formulation many include one or more the FH*/Fc fusion proteins of the present disclosure as well as one or more additional active agents, such as e.g., an antibacterial drug.

Administration of a FH6-7-linker-Fc or FH*-linker-Fc fusion protein composition, or pharmaceutical formulation in accordance with the method of treatment provides a microbiocidal therapeutic effect that protects the subject from and/or treats the progression of pathogenic microbe infection or microbe-mediated disease. In some embodiments, the method of treatment can further comprise administration of one or more additional therapeutic agents or treatments known to those of skill in the art to prevent and/or treat the microbe infection or associated disease or condition. Such methods comprising administration of one or more additional agents can encompass combined administration (where two or more therapeutic agents are included in the same or separate formulations), and separate administration, in which case, administration of the fusion protein composition or formulation can occur prior to, simultaneously, and/or following, administration of the additional therapeutic agent.

In some embodiments of the methods of treatment of the present disclosure, the FH6-7-linker-Fc or FH*-linker-Fc fusion protein composition or pharmaceutical formulation is administered to a subject by any mode of administration that delivers the agent systemically, or to a desired target tissue. Systemic administration generally refers to any mode of administration of the fusion protein into a subject at a site other than directly into the desired target site, tissue, or organ, such that the fusion protein or formulation thereof enters the subject's circulatory system and, thus, is subject to metabolism and other like processes. Accordingly, modes of administration useful in the methods of treatment of the present disclosure can include, but are not limited to, injection, infusion, instillation, and inhalation. Administration by injection can include intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebro spinal, and intrasternal injection and infusion. Additionally, in some embodiments, a pharmaceutical formulation of the FH6-7-linker-Fc or FH*-linker-Fc fusion protein is formulated such that the fusion protein is protected from inactivation in the gut. Accordingly, the method of treatments can comprise oral administration of the formulation.

For the prevention or treatment of microbial infection or associated disease or condition, the appropriate dosage of the FH6-7-linker-Fc or FH*-linker-Fc fusion protein contained in the compositions and formulations of the present disclosure (when used alone or in combination with one or more other additional therapeutic agents) will depend on the specific microbial infection, disease, or condition being treated, the severity, and the course of the disease, whether the fusion protein is administered for preventive or therapeutic purposes, the previous therapy administered to the patient, the patient's clinical history and response to the fusion protein, and the discretion of the attending physician. The FH6-7-linker-Fc or FH*-linker-Fc fusion protein included in the compositions and formulations described herein, can be suitably administered to the patient at one time, or over a series of treatments. Various dosing schedules including but not limited to single or multiple administrations over various time-points, bolus administration, and pulse infusion are contemplated herein.

Depending on the type and severity of the infection or associated disease or condition, about 1 μg/kg to 20 mg/kg of FH6-7-linker-Fc or FH*-linker-Fc fusion protein in a formulation of the present disclosure is an initial candidate dosage for administration to a human subject, whether, for example, by one or more separate administrations, or by continuous infusion. Generally, the administered dosage of the FH6-7-linker-Fc or FH*-linker-Fc fusion protein can be in the range from about 0.05 mg/kg to about 20 mg/kg. In some embodiments, one or more doses of about 0.5 mg/kg, 2.0 mg/kg, 4.0 mg/kg 10 mg/kg, 20 mg/kg, or a range between any two foregoing values (or any combination thereof) may be administered to a human subject. In some embodiments, a dose administered to a human subject can be greater than about 20 mg/kg.

Dosage administration can be maintained over several days or longer, depending on the condition of the subject, for example, administration can continue until the microbial infection or associated disease is sufficiently treated, as determined by methods known in the art. In some embodiments, an initial higher loading dose may be administered, followed by one or more lower doses (e.g., one or more maintenance doses). However, other dosage regimens may be useful. The progress of the therapeutic effect of dosage administration can be monitored by conventional techniques and assays.

Bacterial Strains. In the following examples, N. gonorrhoeae bacterial strains F62 (Shafer et al. 1984), Ctx-r(Spain) (similar to strain F89) (Camara et al. 2012), H041 (also known as World Health Organization reference strain X) (Ohnishi et al. 2011, Unemo et al. 2016), MS11 (Schneider et al. 1991), UMNJ60_06 UM (NJ-60) (Chakraborti et al. 2016), and FA1090 (Hitchcock et al. 1985). Strains Ctx-r(Spain), H041, and NJ-60 are resistant to ceftriaxone. Opacity protein (Opa)-negative mutants of FA1090 (Lewis et al. 2008) (all opa genes deleted) were also used as described below. Experiments described below show in vitro complement-mediated killing of B. burgdorferi strains B31-5A4 and 297 and B. afzelii strain VS461, B. garinii (Bg) (strain ZQ1) and B. bavariensis (Bbav)(strain PBi) and Borrelia miyamotoi (Bm) (strains Fr64b and LB-2001) with the FHFc of FH*/Fc fusions disclosed herein. The serum-sensitive Bb strain B313 (defective in FH binding and killed by serum concentrations >20%) is used as a positive control in some examples herein.

As described more fully herein below in the examples, nucleotide sequence encoding human FH 18-20 (GenBank accession no. NP_000177) (aa 1048-1231, incorporating the D1119G mutation (Jokiranta et al. 2006)), designed to employ optimal codon usage for expression in Nicotiana benthamiana, was synthesized by GENEWIZ (South Plainfield, NJ). This sequence (and the encoded protein fragment) was designated FH*.

The synthetic FH* sequence was cloned into the plant binary expression vector pTRAkc (Maclean et al. 2007) upstream and in-frame with codon-optimized hinge and Fc sequences from human IgG1 (hFc1) and downstream of the signal peptide of the murine mAb24 heavy-chain (Iph) (Voss et al. 1995). Additional clones encoding N-terminal amino acid extensions to the FH* sequence or linkers between FH* and Fc were made using overlap extension PCR.

The present disclosure also provides additional variants including those having the following amino terminal amino acids TS (threonine, and serine), DTS (aspartic acid, threonine and serine) and RDTS (arginine, aspartic acid, threonine, and serine) at the N terminus of FH* in the constructs having the following linear structure when read from the amino terminus to the carboxy terminus of the amino acid sequence: amino-terminal amino acids-FH*-linker(s)-Fc-carboxy terminus. Examples of variants having such additional amino-terminal amino acids are listed in Table 1 as strains S2477, S2493 S2479, S2481 and S2499.

In an additional aspect of the invention, there is provided an alternative ordering of the elements of these clones in which the constructs reverse the order of elements in the linear structure when read from the amino terminus to the carboxy terminus of the amino acid sequence: Amino-terminal hFc-linker—additional amino acids amino terminal to FH(D18-20). Examples of clones having this reversed order are listed in Table 1 as S2507, S2509, S2534 and S2635. The FH/Fc fusion protein molecular constructs that were assembled are listed in Table 1. Throughout the specification, these are referred to by Agrobacterium tumefaciens strain number. The polynucleotide sequences of these constructs and the encoded amino acid sequences of the FH/Fc fusion proteins are provided in the Examples below, and the accompanying figures and Sequence Listing.

TABLE 1
Description of plant-produced FH*/Fc fusion protein molecules

| Strain | | | |
|---|---|---|---|
| Fusion | | | |
| Protein | Plas- | Binary Expression Vector | |
| Name | mid | Name | Modifications |
| S2366 | p1338 | pTRAk-c-lph-FH*- | AAAGGSS linker |
| | | (AAAGGSS)-hFc1 | |
| S2368 | p1339 | pTRAk-c-lph-FH*-(G₄S)₂-hFc1 | (GGGGS)₂ linker |
| S2370 | p1340 | pTRAk-c-lph-FH*-(G₄S)₃-hFc1 | (GGGGS)₃ linker |
| S2381 | p1346 | pTRAk-c-lph-FH*-hFc1 | no linker |
| S2417 | P1364 | pTRAk-c-lph-FH(6-7)-(G₄S)₃- | N-terminal FH(6-7)- |
| | | hFc1 | (GGGGS)₃-hFc1 |
| S2479 | p1395 | pTRAk-c-lph-(DTS)FH*- | DTS and (GGGGS)₂ |
| | | (G₄S)₂-hFc | linker |
| S2481 | p1396 | pTRAk-c-lph-(RDTS)FH*- | RDTS and |
| | | (G₄S)₂-hFc | (GGGGS)₂ linker |
| S2477 | p1394 | pTRAk-c-lph-(TS)FH*- | N-terminal TS |
| | | (G₄S)₂-hFc1 | |
| S2493 | p1404 | pTRAk-c-lph-(TS)FH*-(G₄S)₂- | N-terminal TS |
| | | hFc1(D270A/K322A) | ‘complement- |
| | | | inactive’ |
| S2499 | p1407 | pTRAk-c-lph-(TS)FH*(G₄S)₂- | N-terminal TS, |
| | | hFc3(IgG1 hinge) | (GGGGS)₂ linker |
| | | | hFc3 with IgG1 |
| | | | hinge |
| S2507 | p1411 | pTRAk-c-lph-hFc1(hr4.1)- | N-terminal hFc1- |
| | | (GGGGS)-(TS)FH* | (GGGGS)-(TS)-FH* |
| S2509 | p1412 | pTRAk-c-lph-hFc1-(G₄S)₂- | N-terminal hFc1 |
| | | (TS)FH* | (GGGGS)₂-(TS)- |
| | | | FH* |
| S2534 | p1425 | pTRAk-c-lph-hFc3-(G₄S)₂- | N-terminal hFc3- |
| | | (TS)FH* | (GGGGS)₂-(TS)- |
| | | | FH* |
| S2538 | P1427 | pTRAk-c-lph-hFc3(IgG1 | S2534 + hFc3 |
| | | hinge)(435H)-(G₄S)₂-(TS)FH* | mutations R1203L/ |
| | | | R1206N/R1210S |
| S2635 | P1475 | pTRAk-c-lph-FH(6-7)-(G₄S)₂- | N-terminal FH(6-7)- |
| | | hFc3(IgG1 hinge)(435H) | (GGGGS)2- |
| | | | hFc3(IgG1 |
| | | | hinge)(435H) |

In addition, with reference to particular amino acids indicated as modifications enumerated below in these examples and Tables, the amino acids are referred to by the standard amino acid single letter abbreviations well-known in the art.

As used herein, the term “Fc” means the CH2-CH3 domains of an IgG1, IgG2, IgG3 or IgG4. Preferably, the foregoing immunoglobulins will be human. In some descriptions herein below the various human Fc are designated “hFcX” where X is the immunoglobulin isotype. The amino acid sequences of these molecules are known to those skilled in the art and are retrievable from a number of well-known protein sequence databases.

In particular, the term “Fc1” as used herein refers to the Fc (CH2-CH3) domains of IgG1 (UniProtKB/Swiss-Prot: P01857.1) which has the amino acid sequence:

(SEQ ID NO: 1)
APELLGGPSVFLFPPKPKDTLMISRTPEVTCVVVD
VSHEDPEVKFNWYVDGVEVHNAKTKPREEQYNSTY
RVVSVLTVLHQDWLNGKEYKCKVSNKALPAPIEKT
ISKAKGQPREPQVYTLPPSRDELTKNQVSLTCLVK
GFYPSDIAVEWESNGQPENNYKTTPPVLDSDGSFF
LYSKLTVDKSRWQQGNVFSCSVMHEALHNHYTQKS
LSLSPGK

The term “Fc3” means the Fc (CH2-CH3) domains of IgG3 (GenBank accession no. CAA67886.1) which has the amino acid sequence:

(SEQ ID NO: 2)
APELLGGPSVFLFPPKPKDTLMISRTPEVTCVVVD
VSHEDPEVQFKWYVDGVEVHNAKTKPREEQYNSTF
RVVSVLTVLHQDWLNGKEYKCKVSNKALPAPIEKT
ISKTKGQPREPQVYTLPPSREEMTKNQVSLTCLVK
GFYPSDIAVEWESSGQPENNYNTTPPMLDSDGSFF
LYSKLTVDKSRWQQGNIFSCSVMHEALHNHFTQKS
LSLSPGK

The term “hinge of IgG1” means the sequence between CH1 and CH2 domains of IgG1 (UniProtKB/Swiss-Prot: P01857.1) which have the amino acid sequences: EPKSCDKTHTCPPCP (SEQ ID NO: 3) (full or long IgG1 hinge) or DKTHTCPPCP (SEQ ID NO: 4) (short IgG1 hinge).

The term “hinge of IgG3” means (the sequence between CH1 and CH2 domains of IgG3 (GenBank accession no. CAA67886.1) which has the amino acid sequence ELKTPLGDTTHTCPRCPEPKSCDTPPPCPRCPEPKSCDTPPPCPRCPEPKSCDTPPPCPRCP (SEQ ID NO: 5) or some portion thereof.

The DNA nucleotide sequence of the foregoing Fc and hinge regions may be determined according to a DNA codon table in which the codons are defined by the DNA of a mammalian cell nucleus. The DNA codons in the table occur on the sense DNA strand and are arranged in a 5′-to-3′ direction. The DNA codon table below can be used to compose a DNA sequence when starting from a known amino acid sequence as provided herein:

Inverse table for the standard genetic code

| Amino | | Amino | |
|---|---|---|---|
| acid | DNA codons | acid | DNA codons |
| Ala, A | GCT, GCC, GCA, GCG | Ile, I | ATT, ATC, ATA |
| Arg, R | CGT, CGC, CGA, CGG; | Leu, L | CTT, CTC, CTA, CTG; |
| | AGA AGG | | TTA, TTG |
| Asn, N | AAT, AAC | Lys, K | AAA, AAG |
| Asp, D | GAT, GAC | Met, M | ATG |
| Asn or | AAT, AAC; GAT, GAC | Phe, F | TTT, TTC |
| Asp, B | | | |
| Cys, C | TGT, TGC | Pro, P | CCT, CCC, CCA, CCG |
| Gln, Q | CAA, CAG | Ser, S | TCT, TCC, TCA, TCG; |
| | | | AGT, AGC |
| Glu, E | GAA, GAG | Thr, T | ACT, ACC, ACA, ACG |
| Gln or | CAA, CAG; GAA, GAG | Trp, W | TGG |
| Glu, Z | | | |
| Gly, G | GGT, GGC, GGA, GGG | Tyr, Y | TAT, TAC |
| His, H | CAT, CAC | Val, V | GTT, GTC, GTA, GTG |
| START | ATG | STOP | TAA, TGA, TAG |

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Preparation of FH*/Fc Fusion Protein Constructs with Variant Structures and Linkers

This example illustrates the preparation of various recombinant plasmid constructs encoding FH*/Fc fusion proteins for expression in Planta, particularly Nicotiana benthiama The constructs include variation in the fusion protein linker structures for analysis of improved biocidal potency.

Material and Methods

A. FH*/Fc Plasmid Construct

Plasmids were constructed to express chimeric proteins comprising human FH domains 18-20 (GenBank accession #P08603) fused to the N-terminus of human IgG1 Fc (GenBank accession #AAD38158). A DNA sequence encoding the FH domain 18-20 was designed by optimizing both the codon usage and mRNA accumulation for expression in N. benthamiana. A D>G amino acid point mutation in domain 19 (FH position 1119) was incorporated to prevent complement activation on human or animal (host) cells (Shaughnessy et al. 2016). This FH D1119G (18-20) polynucleotide sequence, designated as “FH”, was synthesized by GENEWIZ. The synthetic FH* polynucleotide sequence was cloned into the PstI and SacI sites of a pTRAkc plant expression vector (Maclean et al., 2007) upstream of and in-frame with a codon-optimized hinge and human IgG1 Fc sequence, and downstream of the signal peptide of the murine mAb24 heavy-chain (GenBank accession #CAA47649.1) (Vaquero et al. 1999). The polynucleotide sequence encoding FH* and a portion of the mAb24 signal peptide is shown below (SEQ ID NO: 6).

(SEQ ID NO: 6)
ctgcaggtgttcactcctgcgtcaacccccccacc
gtgcagaacgcctacatcgtgtcccggcagatgag
caagtacccctccggtgagagggtgagataccagt
gccgttccccctacgagatgttcggggacgaggag
gtgatgtgcctcaacggtaactggaccgagccccc
ccagtgcaaggactccaccggtaagtgtggacccc
ccccccccatcgacaacggcgggatcacctccttc
ccgctgtccgtctacgccccggcctcctccgtcga
gtatcagtgccagaacctgtaccagctcgagggca
acaagaggatcacctgccgtaacggtcagtggtcc
gagccccccaagtgcctccatccctgcgtgatcag
ccgtgagatcatggagaactacaacatcgccctgc
ggtggaccgccaagcagaagctctactccaggacc
ggcgagagcgtcgagtttgtgtgcaagcgtggtta
ccggctctcctccaggtcccataccctcaggacca
cctgctgggacggcaagctcgagtaccccacctgt
gccaagagggagctc

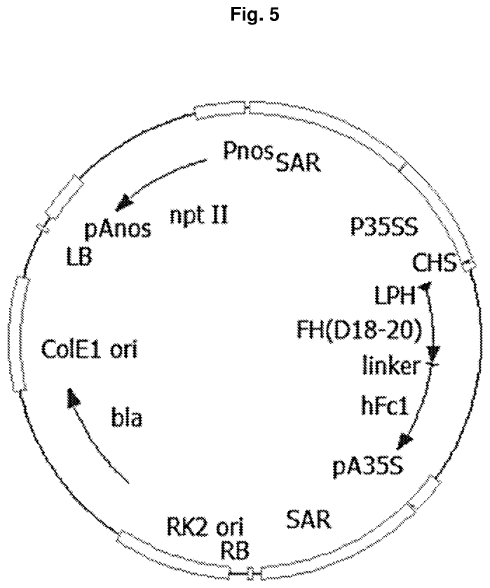

A map of the FH*/Fc plasmid construct in the pTRAkc plant expression vector is depicted FIG. 5. The open reading frame (ORF) is between the CHS enhancer and the 35S polyadenylation signal (pA35S).

B. FH*/Fc Variant Linker Constructs

Additional clones encoding variant N-terminal amino acid extensions to the FH* sequence, or variants of the linker between FH* and Fc were prepared using overlap extension PCR, as described in further detail below.

Four variants of the FH*/Fc fusion protein with different linkers between FH* and Fc: (i) no linker (plasmid 1346 and strain S2381) (aa sequence of SEQ ID NO: 14); (ii) linker AAAGG (plasmid p1338 and strain S2366) (aa sequence of SEQ ID NO: 8); (iii) linker (GGGGS)₂ (plasmid p1339 and strain S2368) (aa sequence of SEQ ID NO: 10); and (iv) linker (GGGGS)₃ (plasmid p1340 and strain S2370) (aa sequence of SEQ ID NO: 12).

Polynucleotide sequences encoding the four different linkers were added to the C-terminus of FH* using PCR (ACCUZYME™ Mix, Bioline), and the resulting FH*-linker sequences were cloned into pTRAkc upstream of Fc as described above.

The resulting plasmids were verified by DNA sequencing and then transformed into electrocompetent Agrobacterium tumefaciens GV3101 (pMP90RK) (Koncz et al. 1986) as described by Shen & Forde (Shen et al. 1989). The resulting A. tumefaciens strains were given strain names, S2366, S2368, S2370, and S2381, which also were used to designate the resulting encoded fusion proteins. The amino acid sequences of the four variant encoded FH*/Fc fusion proteins are also shown below with their different variant linkers depicted in bold type.

S2366: pTRAk-c-Iph-FH*-(AAAGGSS)-hFc1
(SEQ ID NO: 8)
MEWSWIFLFLLSGTAGVHSCVNPPTVQNAYIVSRQ
MSKYPSGERVRYQCRSPYEMFGDEEVMCLNGNWTE
ISREIMENYNIALRWTAKQKLYSRTGESVEFVCKR
GYRLSSRSHTLRTTCWDGKLEYPTCAKRAAAGGSS
EPKSCDKTHTCPPCPAPELLGGPSVFLFPPKPKDT
LMISRTPEVTCVVVDVSHEDPEVKFNWYVDGVEVH
NAKTKPREEQYNSTYRVVSVLTVLHQDWLNGKEYK
CKVSNKALPAPIEKTISKAKGQPREPQVYTLPPSR
DELTKNQVSLTCLVKGFYPSDIAVEWESNGQPENN
YKTTPPVLDSDGSFFLYSKLTVDKSRWQQGNVFSC
SVMHEALHNHYTQKSLSLSPGK
S2368: pTRAK-c-Iph-FH*-(GGGGS)₂-hFc1
(SEQ ID NO: 10)
MEWSWIFLFLLSGTAGVHSCVNPPTVQNAYIVSRQ
MSKYPSGERVRYQCRSPYEMFGDEEVMCLNGNWTE
PPQCKDSTGKCGPPPPIDNGGITSFPLSVYAPASS
VEYQCQNLYQLEGNKRITCRNGQWSEPPKCLHPCV
ISREIMENYNIALRWTAKQKLYSRTGESVEFVCKR
GYRLSSRSHTLRTTCWDGKLEYPTCAKRGGGGSGG
GGSSEPKSCDKTHTCPPCPAPELLGGPSVFLFPPK
PKDTLMISRTPEVTCVVVDVSHEDPEVKFNWYVDG
VEVHNAKTKPREEQYNSTYRVVSVLTVLHQDWLNG
KEYKCKVSNKALPAPIEKTISKAKGQPREPQVYTL
PPSRDELTKNQVSLTCLVKGFYPSDIAVEWESNGQ
PENNYKTTPPVLDSDGSFFLYSKLTVDKSRWQQGN
VFSCSVMHEALHNHYTQKSLSLSPGK
S2370: pTRAk-c-Iph-FH*-(GGGGS)₃-hFc1
(SEQ ID NO: 12)
MEWSWIFLFLLSGTAGVHSCVNPPTVQNAYIVSRQ
MSKYPSGERVRYQCRSPYEMFGDEEVMCLNGNWTE
PPQCKDSTGKCGPPPPIDNGGITSFPLSVYAPASS
VEYQCQNLYQLEGNKRITCRNGQWSEPPKCLHPCV
ISREIMENYNIALRWTAKQKLYSRTGESVEFVCKR
GYRLSSRSHTLRTTCWDGKLEYPTCAKRGGGGSGG
GGSGGGGSSEPKSCDKTHTCPPCPAPELLGGPSVF
LFPPKPKDTLMISRTPEVTCVVVDVSHEDPEVKFN
WYVDGVEVHNAKTKPREEQYNSTYRVVSVLTVLHQ
DWLNGKEYKCKVSNKALPAPIEKTISKAKGQPREP
QVYTLPPSRDELTKNQVSLTCLVKGFYPSDIAVEW
ESNGQPENNYKTTPPVLDSDGSFFLYSKLTVDKSR
WQQGNVFSCSVMHEALHNHYTQKSLSLSPGK
S2381: pTRAk-c-Iph-FH*-hFc1
(SEQ ID NO: 14)
MEWSWIFLFLLSGTAGVHSCVNPPTVQNAYIVSRQ
MSKYPSGERVRYQCRSPYEMFGDEEVMCLNGNWTE
PPQCKDSTGKCGPPPPIDNGGITSFPLSVYAPASS
VEYQCQNLYQLEGNKRITCRNGQWSEPPKCLHPCV
ISREIMENYNIALRWTAKQKLYSRTGESVEFVCKR
GYRLSSRSHTLRTTCWDGKLEYPTCAKRSSEPKSC
DKTHTCPPCPAPELLGGPSVFLFPPKPKDTLMISR
TPEVTCVVVDVSHEDPEVKFNWYVDGVEVHNAKTK
PREEQYNSTYRVVSVLTVLHQDWLNGKEYKCKVSN
KALPAPIEKTISKAKGQPREPQVYTLPPSRDELTK
NQVSLTCLVKGFYPSDIAVEWESNGQPENNYKTTP
PVLDSDGSFFLYSKLTVDKSRWQQGNVFSCSVMHE
ALHNHYTQKSLSLSPGK

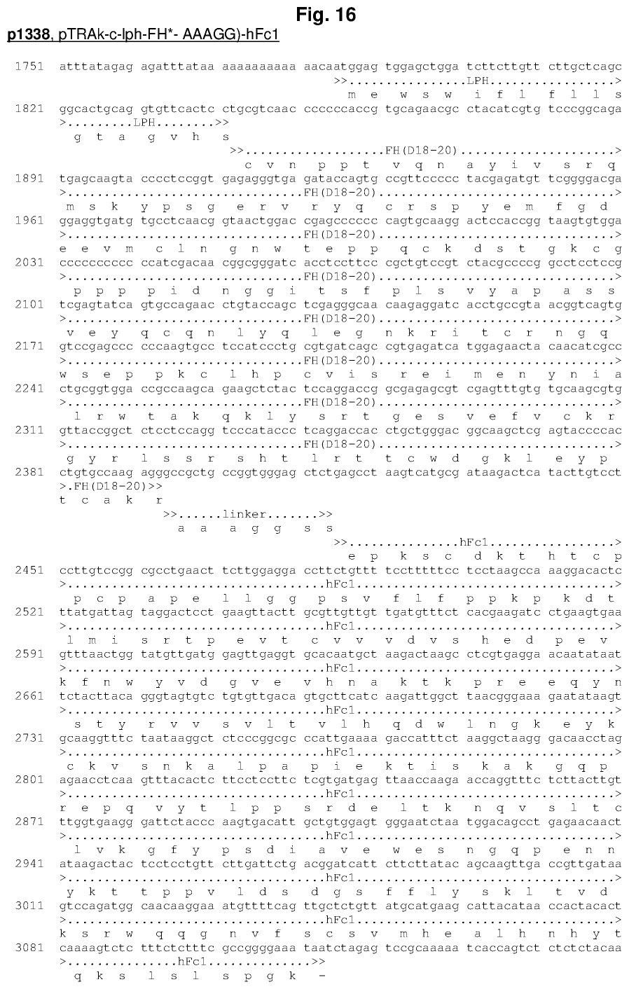

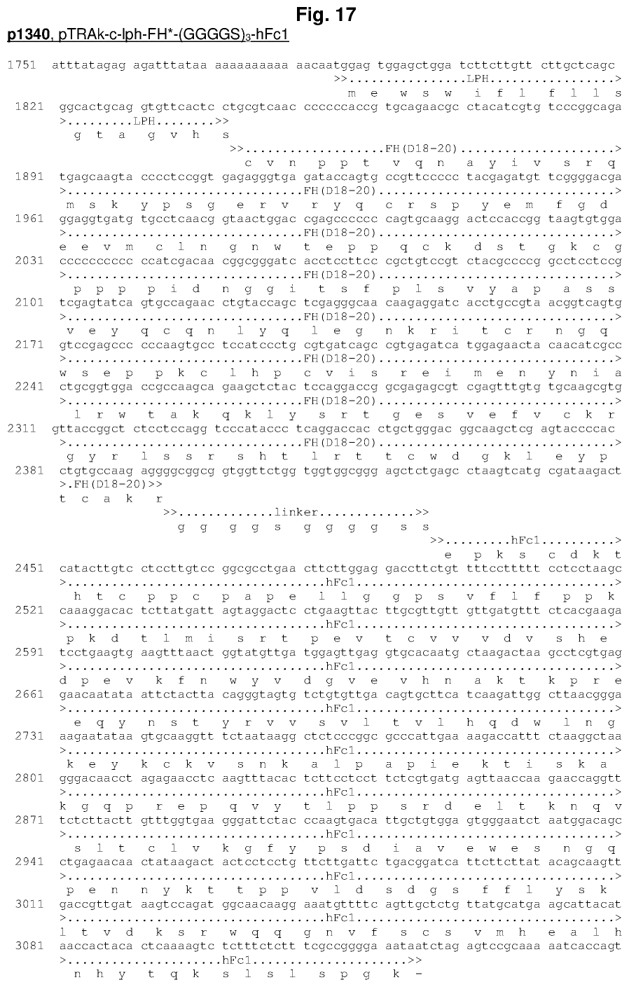

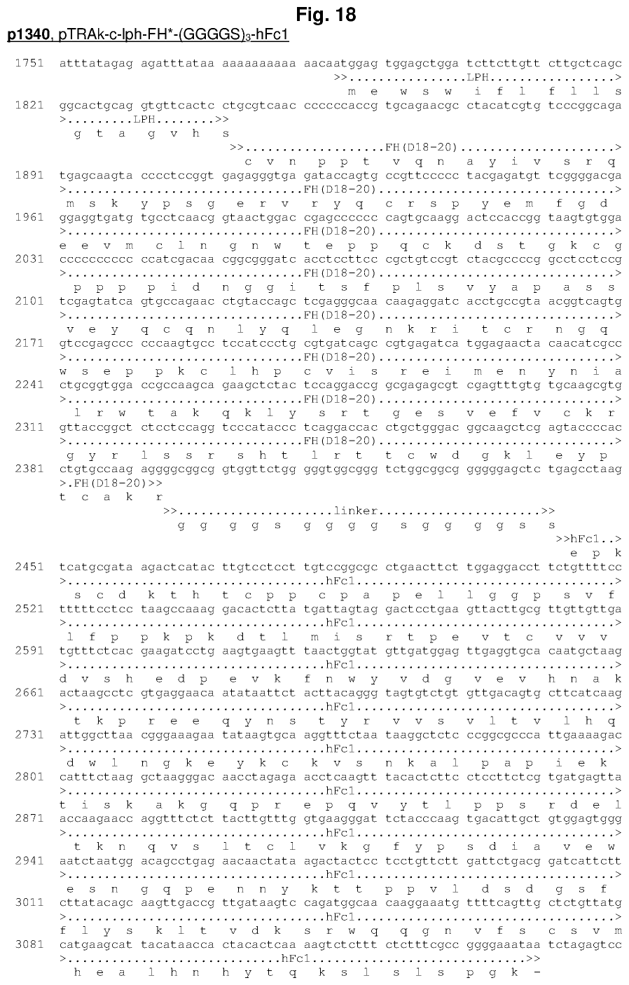

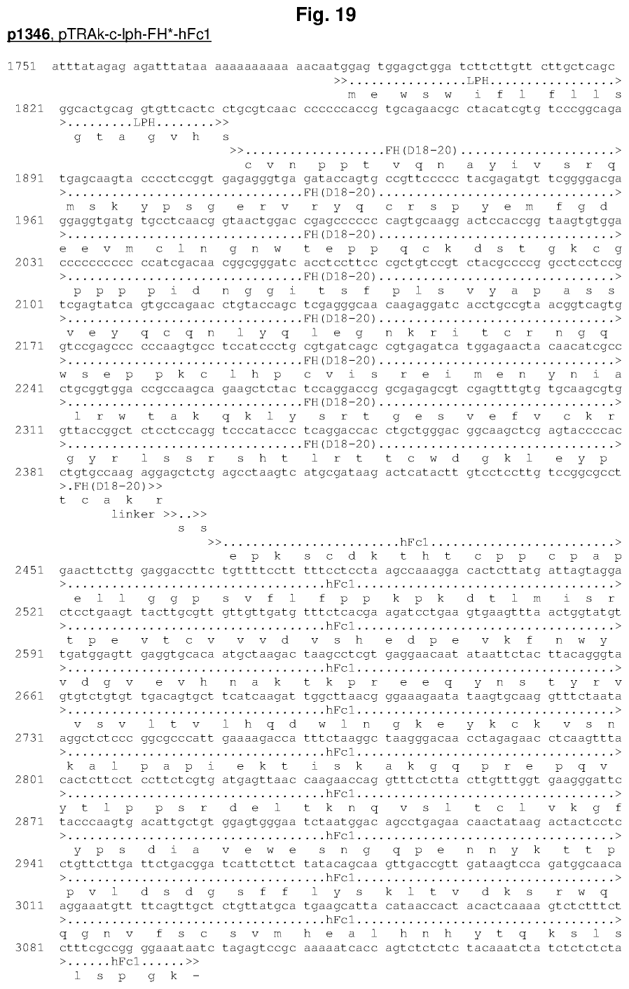

The general description of the structure of these plant-produced FH*/Fc fusion protein molecules are also provided in Table 1. The polynucleotide sequences encoding these four variants S2366 (SEQ ID NO: 7), S2368 (SEQ ID NO: 9), S2370 (SEQ ID NO: 11), and S2381 (SEQ ID NO: 13), as cloned in the plasmids, p1338, p1339, p1340, and p1346, respectively, are shown aligned with the encoded amino acid sequences in FIGS. 16, 17, 18, and 19.

C. FH*/Fc Variant Position Constructs

Additional clones encoding variant N-terminal amino acid extensions to the FH* sequence, or variants of the linker between FH* and Fc were prepared using overlap extension PCR, as described in further detail below.

As summarized in Table 1, in variant S2509, the fusion protein has Fc1 at the N-terminal end and FH* at the C-terminal position (pTRAk-c-Iph-hFc1-(G₄S)₂-(TS)-FH*) which is the reverse of the linear structure of variant S2477 (pTRAk-c-Iph-(TS)FH*-(G₄S)₂-hFc1). Unexpectedly, the S2509 variant fusion protein has greater potency than the S2477 variant fusion protein in the serum microbicidal assay described below in Example 7. The S2507 variant fusion protein has a similar linear structure to S2509 but with a shorter linker: pTRAk-c-Iph-hFc1(GGGGS)-(TS) FH*.

Another additional variant replaced the IgG1 CH2 and CH3 domains with a codon-optimized sequence encoding the corresponding domains of human IgG3 (GenBank accession no. CAA67886.1), with the R at position 435 (Eu numbering) replaced with H, conferring both longer in vivo half-life and Protein A binding (Fc3(435H)). The S2499 (or FH*-Fc3) variant fusion protein (encoded by plasmid construct p1407) has the linear structure: pTRAk-c-Iph-(TS)FH*-(GGGGS)₂-hFc3(IgG1 hinge). The sequence of the IgG1 hinge in this construct is truncated to eliminate the first 5 amino acids of the IgG1 hinge (EPKSC) so that the amino sequence of the preferred IgG1 hinge begins at its N-terminal end with the amino acid residues DKTHTC.

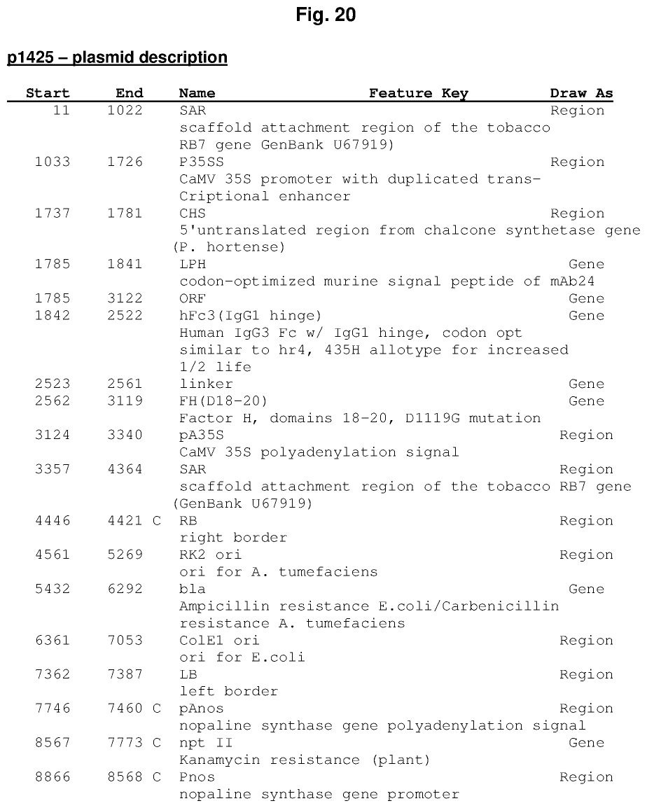

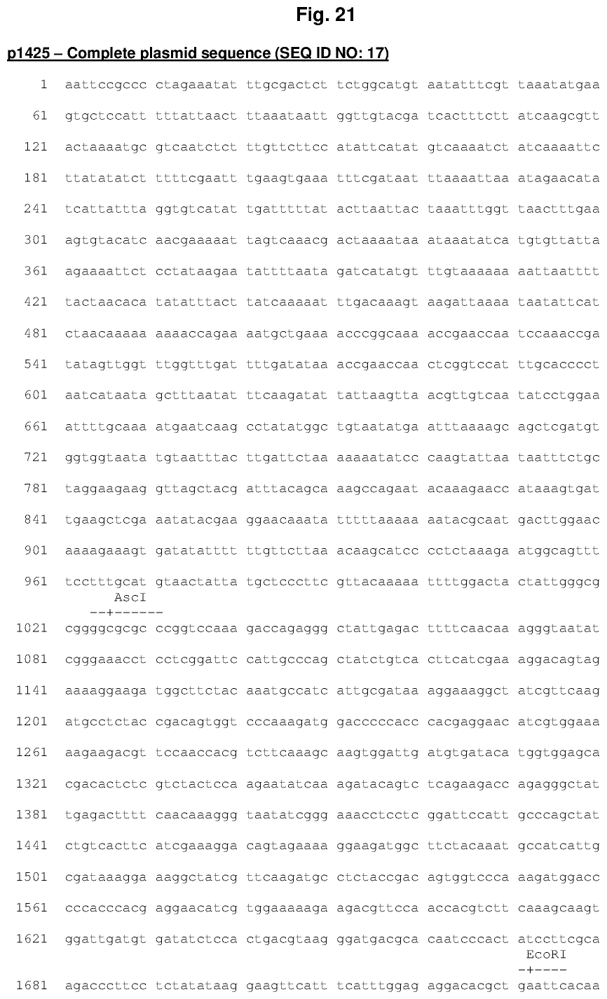

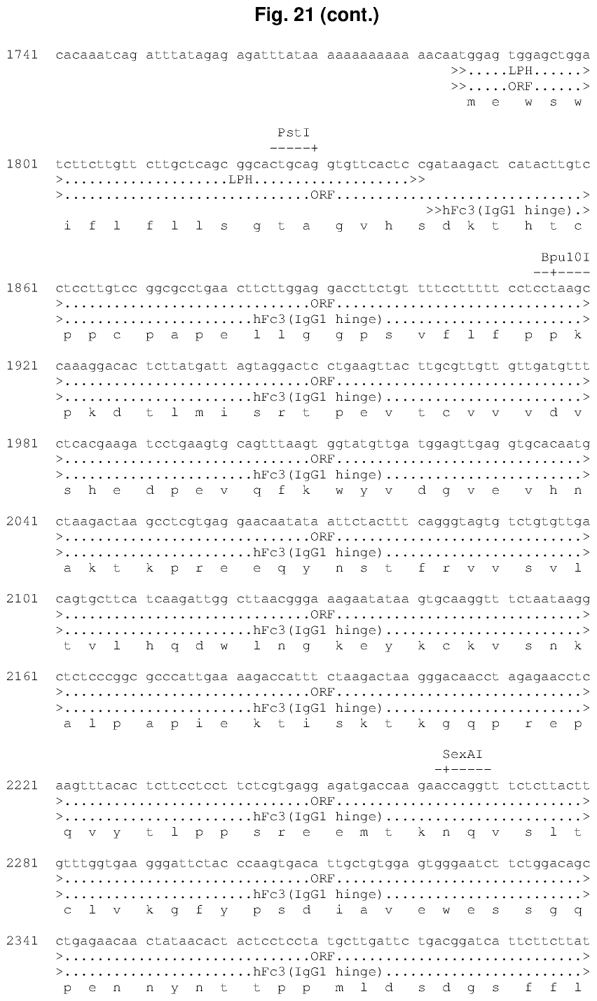

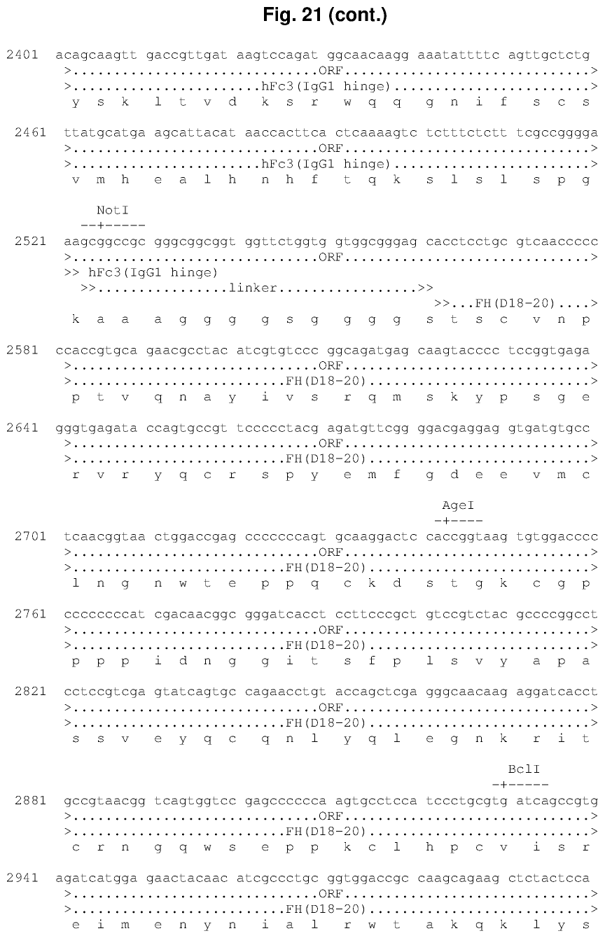

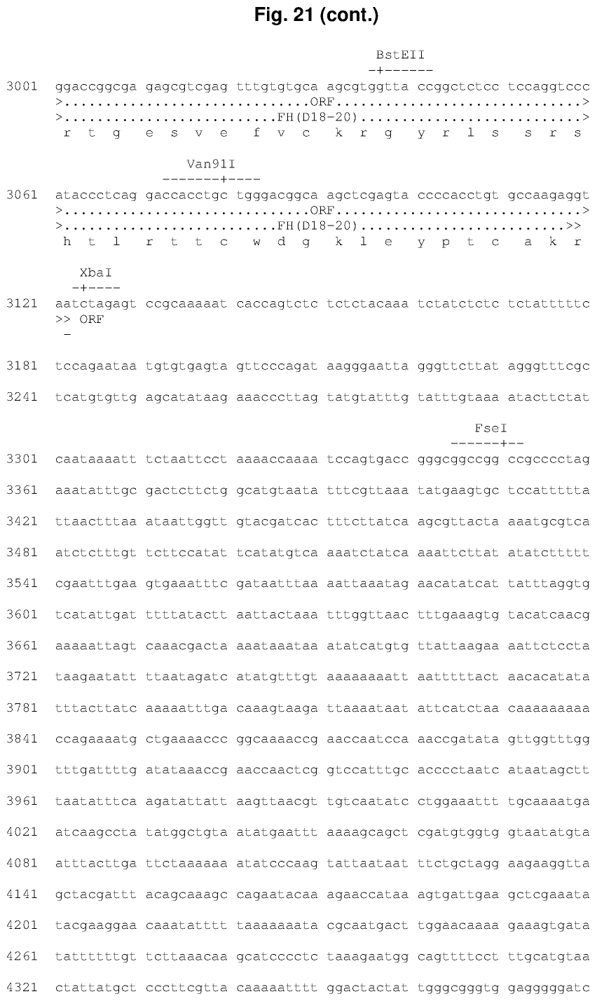

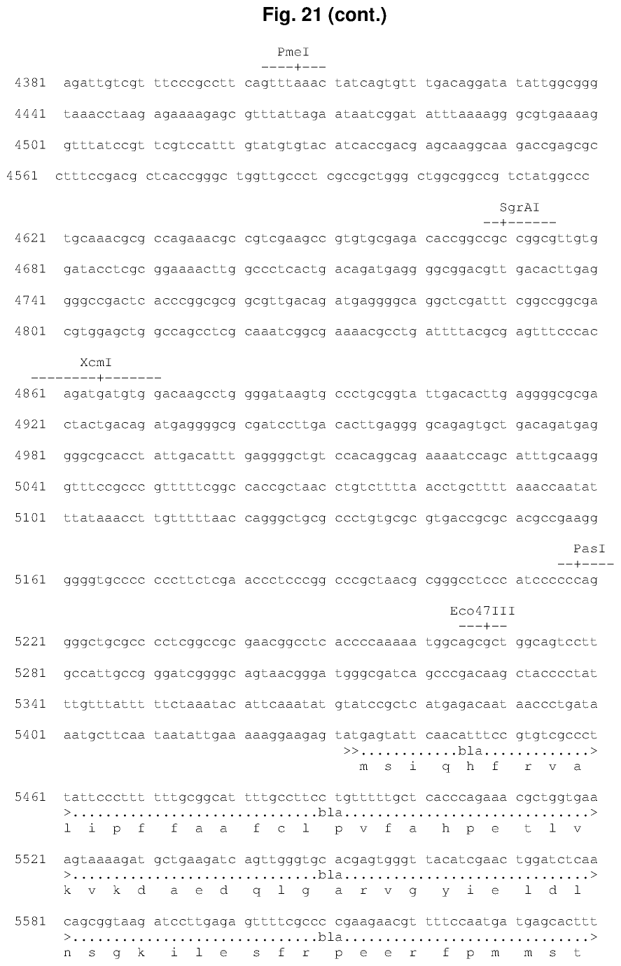

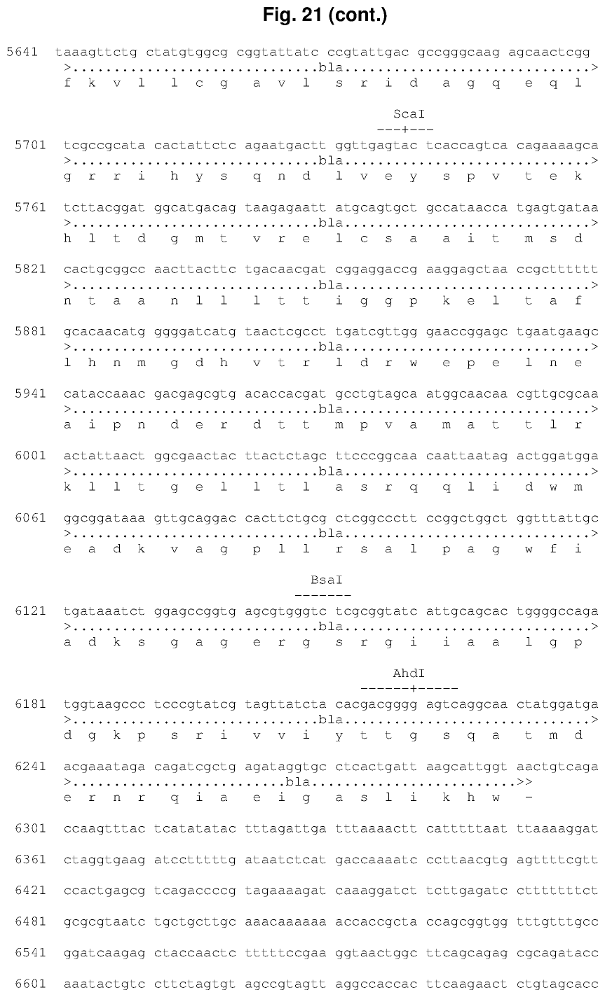

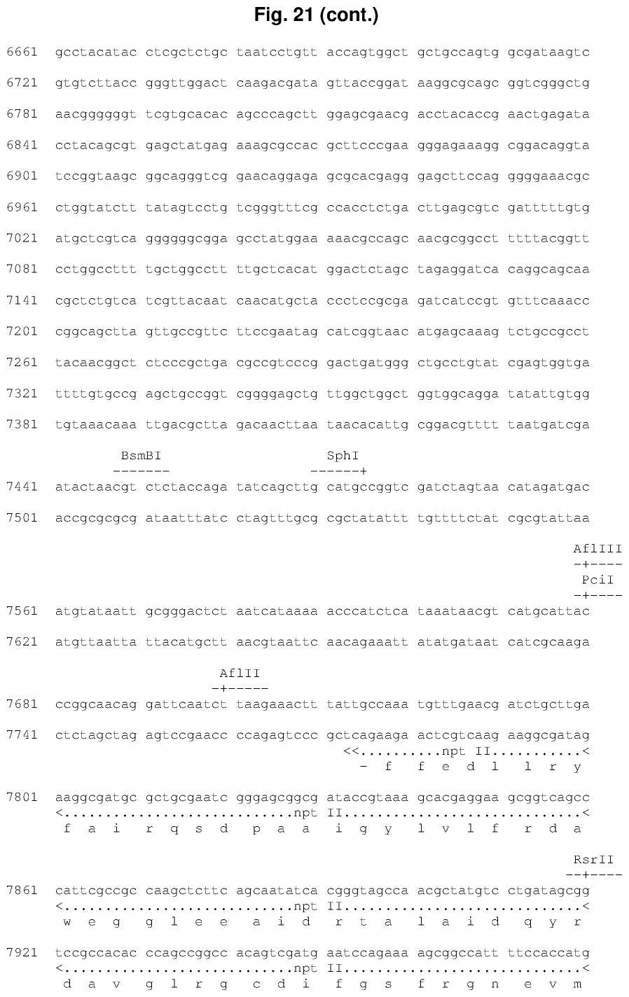

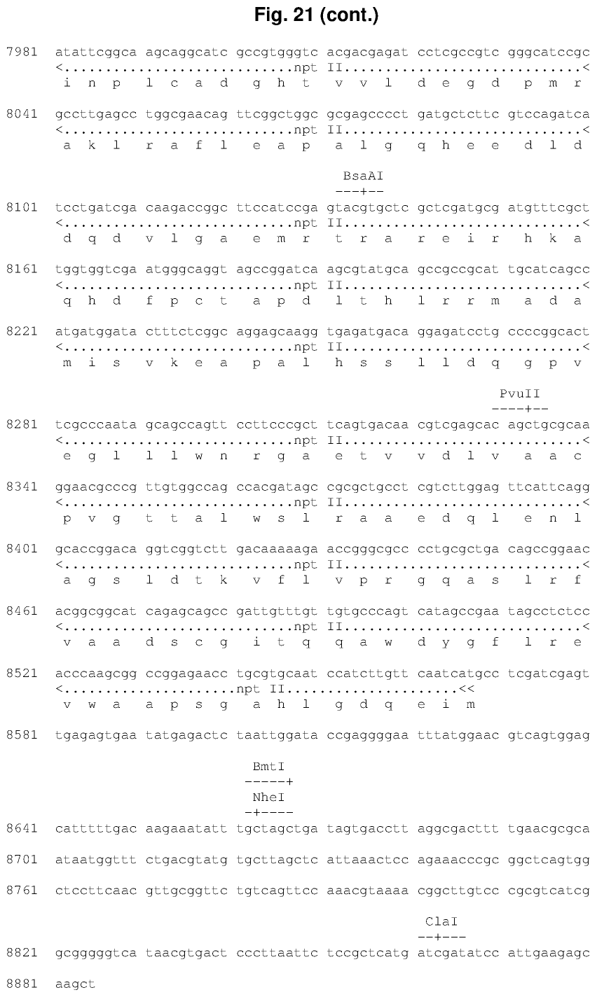

Another type of variant fusion protein constructed has reversed positions of Fc3(435H) and FH*, encoding a protein with Fc3 at the N-terminal end and FH* at the C-terminal. The plasmid encoding this construct was named p1425 and has the linear structure: pTRAk-c-Iph-(IgG1 hinge)hFc3(GGGGS)₂-(TS)FH*. The organization of p1425 construct is summarized in FIG. 20, and the complete encoding polynucleotide sequence thereof, including the PTRAk sequence and all inserts in the open reading frame are shown in FIG. 21 and provided in the accompanying Sequence Listing as SEQ ID NO: 17.

The variant fusion protein S2534 (or “Fc3-FH*”) produced by this plasmid p1425 was shown to dramatically enhance complement-mediated killing of Neisseria gonorrhoeae. The amino acid sequence of the encoded FH/Fc fusion protein are also shown below as SEQ ID NO: 16 with the linker depicted in bold type. The encoding polynucleotide sequence cloned in p1425 is provided herein as SEQ ID NO: 15.

S2534, pTRAk-c-Iph-hFc3(IgG1 hinge)
(435H)-(GGGGS)₂-(TS)FH*
(SEQ ID NO: 16)
MEWSWIFLFLLSGTAGVHSDKTHTCPPCPAPELLG
GPSVFLFPPKPPCPAPELLGGPSVFLFPPKPKDTL
MISRTPEVTCVVVDVSHEDPEVQFKWYVDGVEVHN
AKTKPREEQYNSTFRVVSVLTVLHQDWLNGKEYKC
KVSNKALPAPIEKTISKTKGQPREPQVYTLPPSRE
EMTKNQVSLTCLVKGFYPSDIAVEWESSGQPENNY
NTTPPMLDSDGSFFLYSKLTVDKSRWQQGNIFSCS
VMHEALHNHFTQKSLSLSPGKAAAGGGGSGGGGST
SCVNPPTVQNAYIVSRQMSKYPSGERVRYQCRSPY
EMFGDEEVMCLNGNWTEPPQCKDSTGKCGPPPPID
NGGITSFPLSVYAPASSVEYQCQNLYQLEGNKRIT
CRNGQWSEPPKCLHPCVISREIMENYNIALRWTAK
QKLYSRTGESVEFVCKRGYRLSSRSHTLRTTCWDG
KLEYPTCAKR

A further plasmid construct, p1407, was prepared having the same structure as p1425 except that the positions of the sequence encoding hFc3(IgG1 hinge) (435H) and -(TS)FH* are in reverse order relative to the linker. Using the full sequence of pTRAk as disclosed in FIG. 21, strategies for producing p1407 from p1425 or p1425 from p1407 will be readily apparent to one skilled in the art of cloning and molecular biology.

### Example 2: Transient Expression of FH6,7-(Linker)-Fc and FH*-(Linker)-Fc in Planta

Transient expression of recombinant proteins was accomplished by whole-plant vacuum infiltration (Fischer et al. 1999) of N. benthamianaΔXT/FT (Strasser et al. 2008) using A. tumefaciens GV3101 (pMP90RK) (Koncz et al. 1986) containing one of the binary expression vectors, co-infiltrated with A. tumefaciens GV3101 (pMP90RK) containing the binary vector pTRAkc-P19 which encodes the post-transcriptional silencing suppressor P19 (Voinnet et al. 2003). Glycoproteins produced in N. benthamiana ΔXT/FT contain almost homogeneous N-glycan species without plant-specific β1,2-xylose and α1,3-fucose residues (Strasser et al., 2008). After infiltration, the plants were maintained in a grow room under continuous light at 25° C. for 5-7 days prior to harvest and protein purification.

### Example 3: Extraction Concentration and Purification of FH*/Fc Protein

Leaves transiently producing the protein of interest were collected 5-7 days after vacuum infiltration and frozen at −80° C. until use.

Purification of FH6,7/Fc and FH*/Fc fusion proteins was accomplished using a protocol previously used with another plant-produced Fc fusion (Wycoff et al. 2011), which incorporates affinity chromatography with Protein A-MabSelect SuRe or PrismA (GE HealthCare). Purified proteins were concentrated to mg/ml using 10 kDa cut-off centrifugal concentrators, buffer exchanged into PBS and rendered sterile by filtration through 0.22 μm PES membrane filters. Protein concentrations were quantified using absorption at 280 nm and extinction coefficients predicted from the amino acid sequences.

In greater detail, extraction and upstream processing consists of grinding and pressing biomass, with an appropriate buffer (such as Tris, digested protein polyamines, ethylenediamine, PBS, pH 5.8-9.5) that maintain the stability and recovery of the FH6-7/Fc and FH*/Fc in order to segregate solids from the product-containing Raw Juice. The Raw Juice may be treated with acid to pH 4.0-5.0 followed by base treatment to pH 7.2-8.5 or flocculated with divalent cations and phosphates and/or polyethyleneimine (PEI) at 0.005-0.1% (w/v) to agglomerate additional solids followed by centrifugation at 4.5-10K RPM for at least 15 min to remove solids and produce a clarified, product-containing liquid (centrate).

For purification, the centrate obtained above is loaded onto Protein A, or other appropriate, affinity chromatography matrix. The column is washed with 10-30 column volumes (CV) wash buffer containing PBS. Elution is carried out with 0.1 M glycine (acetic acid or citrate may also be used), 0.075-0.3 M NaCl, pH 2.0-3.0 and neutralized with 1 M HEPES, pH 8.0 or 1 M Tris, pH 7.5-8.5 (eluate). The eluate may be further purified via heparin affinity chromatography and eluted via a salt gradient and/or via ion exchange chromatography and eluted via a salt or pH gradient. The polished eluate is buffer exchanged into the final formulation buffer and/or treated to remove endotoxin through a ToxinEraser (GenScript) column. Other excipients may be added to the final formulation to enhance stability and/or potency. The buffer exchanged eluate may be concentrated to the desired protein concentration and filtered through a 0.1-0.2 micron PES membrane prior to storage at or below −65° C.

Alternatively, the Protein A column is washed with 5-10 CV wash buffer containing 1% detergent (4 parts Triton X 114 to 1 part Triton X 100) in PBS. A second wash consist of 5-10 CV of 0.25 M arginine, pH 9.0. Lastly, 20 CV of PBS is used to wash away residual Polymixin B and/or detergent from the column prior to elution. Elution is carried out with 0.05-0.1 M glycine, 0.075-0.15 M NaCl, 10% glycerol, pH 2.0-3.5 and neutralized with 1 M HEPES, pH 8.0 or 1 M Tris, pH 7.5-8.5. The column may also be eluted using 0.1 M acetic acid, 0.007 M lactic acid, 0.036 M sodium lactate, 0.004 M sodium acetate, 0.292 M sucrose, 0.077 M NaCl, pH 3.5 or 0.05-0.1 M citrate, 0.075-0.15 M NaCl, 10% glycerol, pH 2.0-3.5 or 0.09-0.1 M sodium phosphate, 0.45-0.5 M NaCl, 9-10% glycerol, pH 2.5-3.5 and neutralized as above or combination thereof. The eluate is buffer exchanged into 3×PBS or other appropriate stabilizing buffer (such as 0.013 M acetic acid, 0.007 M lactic acid, 0.036 M sodium lactate, 0.004 M sodium acetate, 0.025 M glucose, 0.077 M NaCl, pH 6.0) via dialysis or diafiltration using 3.5-50 kDa cut-off regenerated cellulose, cellulose ester, or polyethersulfone (PES) membranes. Other excipients may be added to the final formulation to enhance stability and/or potency. The buffer exchanged eluate may be concentrated to the desired protein concentration and filtered through a 0.1-0.2 micron PES membrane prior to storage at or below −65° C.

### Example 4: Protein Analysis

Purified protein samples were analyzed using standard methods as follows. Samples were subjected to SDS-polyacrylamide gel electrophoresis (under reducing and non-reducing conditions) on 4-20% Mini-PROTEAN® TGX Stain-Free™ Protein Gels (Bio-Rad, Hercules, CA). Alkaline phosphatase conjugated anti-human IgG (Southern Biotechnology) was used in Western blots a dilution of 1:1000 in PBS with 5% non-fat dry milk. Gel images were obtained using a Bio-Rad Gel Doc EZ imaging system.

### Example 5: Improved Production and Recovery of Intact FH*/Fc Fusion Protein Molecules in Nicotiana benthamiana

One variant (S2366) included an AAAGG linker between FH* and Fc, resulting in the same protein that had previously been expressed in CHO cells (Shaughnessy et al. 2016). Three new FH*/hFc variant fusion proteins as described in Table 1 herein above were produced containing either no linker (S2381) or two or three copies of a GGGGS linker, specifically (GGGGS)₂ and (GGGGS)₃ linker (S2368 and S2370, respectively). Yield of these proteins following Protein A affinity chromatography ranged from 300-600 mg per kg plant fresh weight (Table 2). The yield of the protein produced as a percentage of protein having intact bands on Coomassie stained gels using the GS linkers or no linker at all was measurably greater than the yield of the same protein using linkers containing alanine and glycine.

TABLE 2

| PROTEIN | S2366 | S2368 | S2370 | S2381 |
|---|---|---|---|---|
| LINKER | AAAGG | (GGGGS)₂ | (GGGGS)₃ | none |
| YIELD (MG/KG) | 615-815 | 400 | 300 | 450 |
| % INTACT BAND | 72% | 83% | 81% | 92% |

### Example 6: Expression and Purification of FH*/Fc Fusion Protein in CHO Cells

Cloning, expression in CHO cells and purification from cell culture supernatants of a chimeric protein comprising human FH* (fused to human IgG1 was carried out as follows:

Briefly, the DNA encoding FH domains 18-20 was cloned into AscI-NotI sites of eukaryotic expression vector pCDNA3 containing the sequence encoding mouse IgG2a Fc (34). The human FH18-20/Fc mutant D1119G, was produced using the QuikChange site—directed mutagenesis kit (Agilent Technologies), according to the manufacturer's instructions using the primer 5′-CACCTATTGACAATGGGGGCATTACTTCATTCCCGTT-3′ (SEQ ID NO: 18).

Where indicated, mouse IgG2a Fc was replaced by human IgG1 Fc as follows.

FH domains 18-20 were amplified using primers:

FH18EcoRI
(SEQ ID NO: 19)
5′-GAATTCGTGTGTGAATCCGCCCACAGTAC-3′
and
FH20hIgG1overlapR
(SEQ ID NO: 20)
5′-GCCGCGGGGGGCGAGCCCAAATCTTGTGACAA -3′

Human IgG1 Fc (InvivoGen) was amplified with primers:

FH20hIgG1overlapF
(SEQ ID NO: 21)
5′-AGCCCAAATCTTGTGACAAAACTCACACATGCCCA-3′
and
HIgG1NheI
(SEQ ID NO: 22)
5′-CGGGTAAATGAGTGCTAGCTGG-3′

The PCR products were then fused together by overlap extension PCR using primers FH18EcoRI and HIgG1 NheI. The final PCR product encoding FH* fused to hIgG1 was digested with EcoRI and NheI and cloned into pFUSE-hIgG1-Fc2 (InvivoGen). The resulting plasmids were verified by DNA sequencing and used to transiently transfect Chinese hamster ovary cells using lipofectin (Life Technologies), according to the manufacturer's instructions. Medium from transfected cells was collected after 2d, and FH*/Fc was purified by passage over protein A—agarose. Protein concentrations were determined using the BCA protein Assay kit (Pierce); mass was determined by Coomassie Blue staining of proteins separated by SDS-PAGE.

### Example 7: Comparison of Binding, Microbiocidal Potency and Opsinophagocytotic Potency of FH*/Fc Having Alanine-Containing Linkers and FH*/Fc Having GS Linkers Containing No Alanine

A. Flow Cytometry Assay

Binding of FH*/Fc fusion proteins to bacteria was measured by flow cytometry as described in (Shaughnessy et al. 2016). Briefly, to detect binding of FH*/Fc fusion protein, organisms incubated with each of the human FH*/Fc fusion proteins described above (100 μg/mL) for 30 min and were fixed by the addition of paraformaldehyde (final concentration, 1%). The organisms were pelleted after incubation for 10 min at room temperature, and each of the bound FH*/Fc fusion proteins was detected by flow cytometry.

Data were acquired on a BD LSR II flow cytometer, and data were analyzed using FlowJo software. Anti-human IgG-FITC was from Sigma-Aldrich and was used at a dilution of 1:100 in HBSS containing 0.15 mM CaCl₂) and 1 mM MgCl₂ (HBSS⁺⁺) and 1% BSA (HBSS⁺⁺/BSA) in these flow cytometry assays.

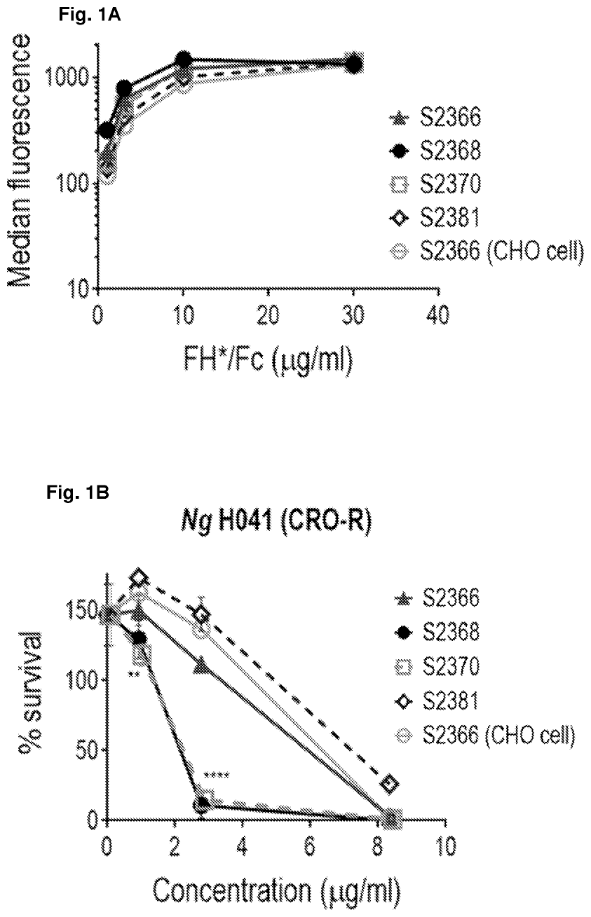

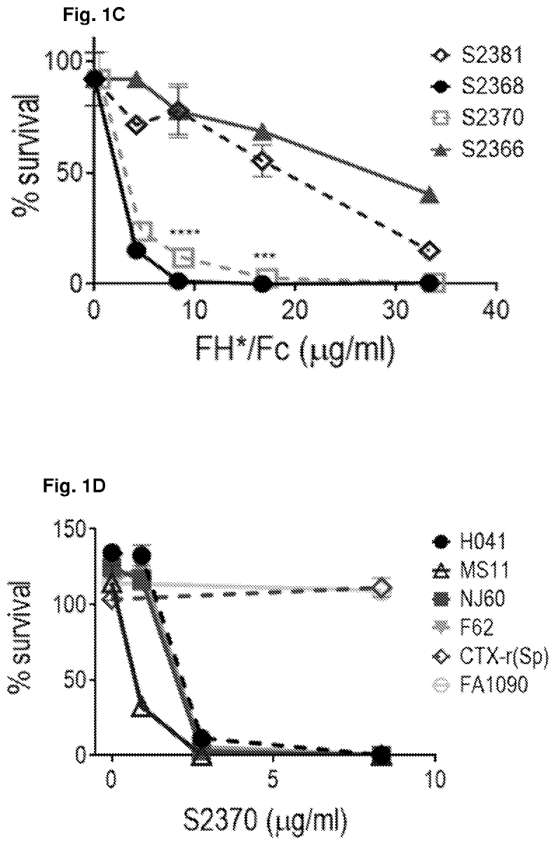

Binding as measured by median fluorescence was determined for four FH*/Fc molecules made in tobacco plants: S2381 FH*/Fc without a linker, or S2366 with AAAGG, two G₄S or three G₄S linkers S2368 and S2370, respectively). FH*/Fc with AAAGG linker made in CHO cells S2366 (CHO cell) was used as a control. A plot of the binding curves is shown in FIG. 1A. As expected, since the fusion proteins all possessed the same FH* sequence they showed similar binding to N. gonorrhoeae strain H041 when tested at dilutions ranging from 1.1 to 30 μg/mL.

B. Serum Microbicidal Assay

Serum microbicidal activity was assessed in bactericidal assays using N. gonorrhoeae H041 bacteria grown in gonococcal liquid media supplemented with CMP-Neu5Ac were performed as described previously (Shaughnessy et al. 2016, Gulati et al. 2019) which are herein incorporated by reference. Approximately 2000 colony forming units (CFUs) of N. gonorrhoeae were incubated with 20% human complement (IgG and IgM depleted normal human serum (Pel-Freez)) in the presence or the absence of the FH*/Fc fusion protein (concentration indicated for each experiment). The final volume of the bactericidal reaction mixture was 150 μL. Aliquots of 25 μL reaction mixtures were plated onto chocolate agar in duplicate at the beginning of the assay (t₀) and again after incubation at 37° C. for 30 min (t₃₀). Survival was calculated as the number of viable colonies at t₃₀ relative to t₀.

As shown by the plots of results depicted in FIG. 1B, the fusion protein variants S2368 (FH*/Fc with (G₄S)₂) and S2370 (FH*/Fc with (G₄S)₃) showed improved complement-dependent microbicidal activity against Ng H041 as compared to S2366 (FH*/Fc with AAAGG) or S2381 (FH*/Fc without a linker). The concentrations required for 50% microbicidal activity (BC₅₀) of S2368 and S2370 were significantly lower than for than S2366 and S2381 (BC₅₀ of 2.1 μg/ml with S2368 and S2370 vs 5.9 and 7.2 μg/mL with S2366 and S2381, respectively). S2366 (CHO cell) or (S2366) produced in planta showed similar microbicidal activity (BC₅₀ of 6.3 and 5.9 μg/mL, respectively). S2381 (no linker) showed the least microbicidal activity.

Further microbicidal testing using S2370 against Ng H041 and five additional gonococcal strains NJ60, F62, MS11, FA1090 and CTX-r(Sp) was carried out using the above-described method. As shown by the results depicted in FIG. 1D, S2370 was lethal against four of the six strains tested H041, NJ60, F62 and MS11, at concentrations under 5 μg/mL, but did not kill FA1090 or CTX-r(Sp)). These six strains showed the same pattern of susceptibility to FH*/Fc with the AAAGG linker produced in CHO cells data not shown.

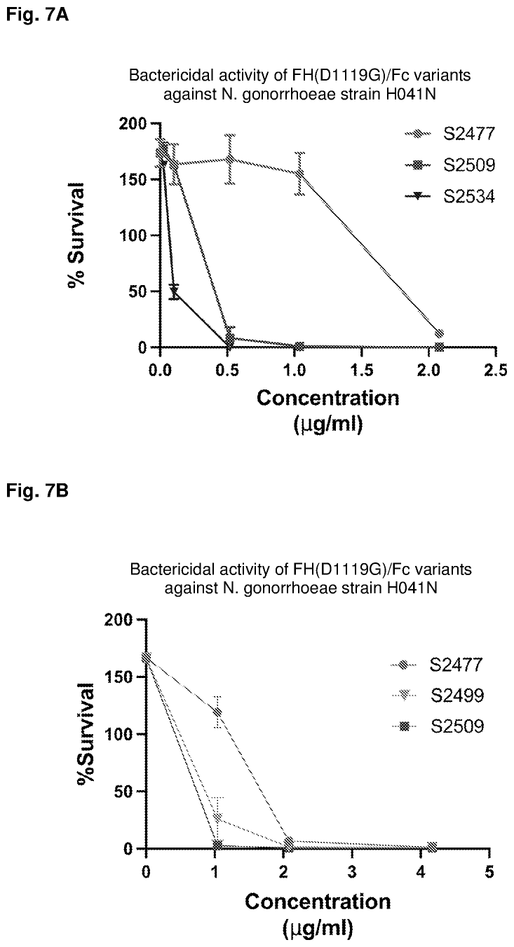

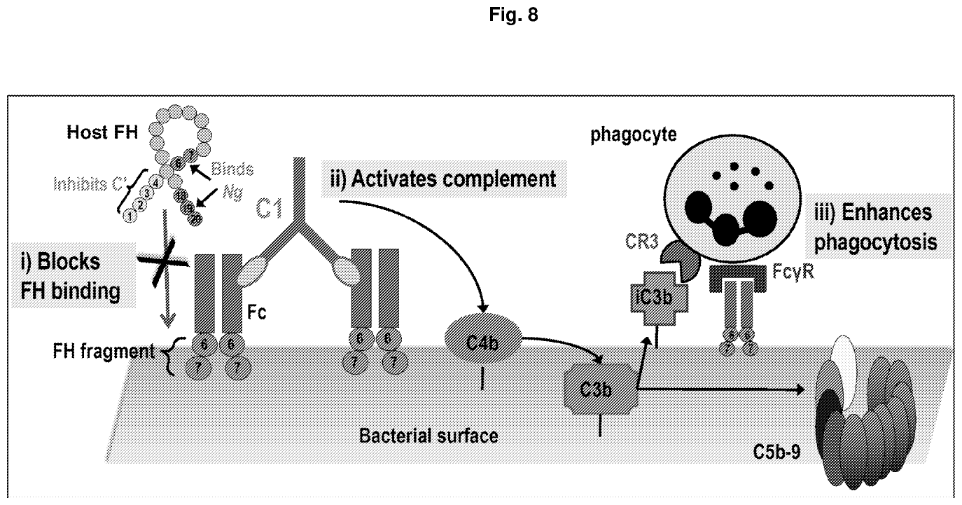

Microbicidal potency of the variant fusion proteins S2509 (N-terminal hFc1-(GGGGS)₂-(TS)-FH*), S2534 (N-terminal hFc3-(GGGGS)₂-(TS)-FH*) and S2477 (N-terminal (TS)-FH*-(GGGGS)₂-hFc1) against Ng H041 was determined using the microbicidal assay described above. As shown by the results plotted in FIG. 7A, the S2509 and S2534 fusion proteins produced in which the Fc is at the N-terminal end of the protein were significantly more potent in the assay than the S2477 fusion protein in which the Fc is at the C-terminal end of the protein.

Microbicidal potency of the fusion proteins S2509 (N-terminal hFc1-(GGGGS)₂-(TS)-FH*), S2499 (N-terminal (TS)-FH*-(GGGGS)₂-hFc3(IgG1 hinge) and S2477 (N-terminal (TS)FH*-(GGGGS)₂-hFc1) was determined using the microbicidal assay described above. As shown by the results plotted in FIG. 7B, the S2509 fusion protein, in which the hFc1 is at the N-terminal end of the protein, was significantly more potent in the assay than the S2477 fusion protein, in which the hFc1 is at the C-terminal end of the protein. The potency of S2499, in which the hFc3(IgG1 hinge) is C-terminal is comparable to that of S2509.

An additional microbiocidal potency assay was carried out using the fusion protein S2534 (hFc3-(GGGGS)₂-(TS)FH*), designated “Fc3-FH”, which was produced in strain number S2534 using plasmid p1425, as described in Example 1. The microbiocidal assay was carried out using a panel of N. gonorrhoeae strains listed in Table 3.

TABLE 3

| WHO 901 | WHO 911 | NJ15 | SD3 | 03701 Cx |
|---|---|---|---|---|
| WHO 902 | WHO 912 | NJ19 | SD5 | PID LS |
| WHO 903 | WHO 913 | NJ26 | SD8 | PID 1 |
| WHO 904 | WHO 914 | NJ27 | SD15 | PID 8 |
| WHO 905 | FA1090 | NJ36 | SF2 | PID 02601 |
| WHO 906 | MS11 | NJ44 | SF6 | PID 333 |
| WHO 907 | F62 | NJ48 | SF7 | PID 6860 |
| WHO 908 | ′252 | NJ60 | WR220 | PID 02201 |
| WHO 909 | NJ1 | OC7 | 1291 | PID 011 |
| WHO 910 | NJ11 | OC14 | 334 | 24-1 |

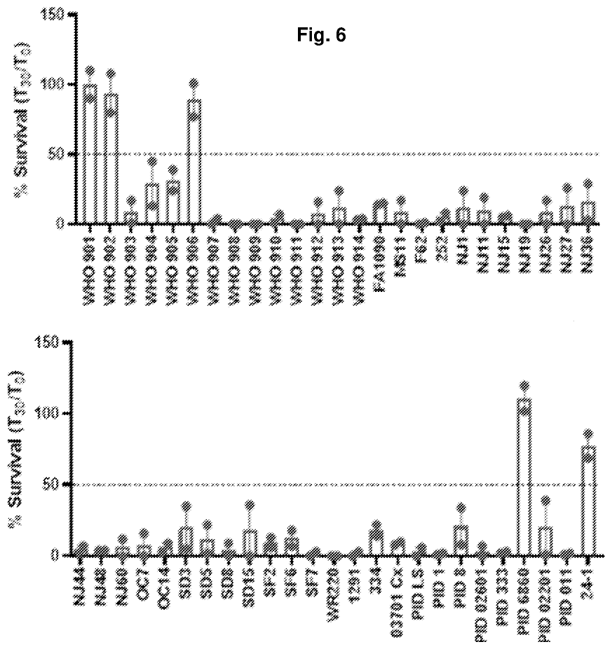

The microbiocidal assay of S2534 was carried out as described above with the following modifications: Fc3-FH* concentration was at (33 μg/mL) and 10% human complement (IgG/IgM depleted serum (Pel-Freez)) was used in a final reaction volume of 150 μL. Results showing the microbiocidal potency of S2534 against the various Ng strains are shown in FIG. 6.

C. Opsonophagocytosis Assay

Opsonophagocytic killing of gonococci with freshly isolated human polymorphonuclear leukocytes (PMNs) was performed as described previously in (Shaughnessy et al. 2016, Shaughnessy et al. 2018) which are herein incorporated by reference. Briefly, heparinized venous blood was obtained from a healthy adult volunteer in accordance with a protocol approved by the Institutional Review Board. PMNs were isolated using Mono-Poly Resolving Medium (MP Biomedicals) according to the manufacturer's instructions. Isolated PMNs were washed and suspended in HBSS without added divalent cations, counted, and diluted to 1×10⁷/ml in HEPES-buffered RPMI 1640 medium supplemented with L-glutamine and 1% heat-inactivated FBS. To measure survival of gonococci in the presence of PMNs, Opacity protein negative (Opa⁻) mutant of N. gonorrhoeae strain FA1090 was added to 1×10⁶ PMNs at a multiplicity of infection of 1 (two bacteria to one PMN). (Opa⁻) N. gonorrhoeae strain FA109 was used, wherein all 11 opa genes have been inactivated to eliminate Opa-CAECAM3 proteins that can serve as ligand for human carcinoembryonic antigen-related cell adhesion molecule 3 (CEACAM3) that is expressed by PMNs and results in phagocytosis (Sarantis et al. 2007). The FH*/Fc fusion protein was added at different concentrations, followed by 10% human complement (Pel-Freez). The reaction mixtures were incubated for 60 min at 37° C. in a shaking water bath. Bacteria were serially diluted and plated at 0 and 60 min on chocolate agar plates. Percentage survival of gonococci in each reaction was calculated as a ratio of CFU at 60 min to CFU at the start of the assay (0 min).

As shown in FIG. 1C, FH*/Fc fusion proteins S2368 and S2370 enhanced PMN-mediated killing significantly more than S2366, containing AAGGG linkers, or S2381 which had no linker. (BC₅₀ of 2.3 and 2.6 μg/mL with S2368 and S2370, as compared to BC₅₀ of 27.4 and 19.1 μg/ml with S2366 and S2381, respectively).

Collectively, the assay data above in this Example 7 showed that S2368 and S2370 ((G₄S)₂ and (G₄S)₃ linkers respectively) improved microbicidal and PMN-mediated opsonophagocytic killing about 2.7- and 11-fold, respectively, compared to S2366 using the AAAGG linker.

### Example 8: In Vivo Activity of HF*/Fc Fusion Protein in Mouse Vaginal Colonization Model of Gonorrhea

This example illustrates a study of the efficacy of the S2370 fusion protein against N. gonorrhoeae in the mouse vaginal colonization model of gonorrhea using FH/C4BP transgenic mice was determined using the methods described above using two N. gonorrhoeae strains that differed in their susceptibility to killing in the human complement-dependent bactericidal assay; sensitive strain H041 and resistant strain FA1090.

A. Mouse Strains

Human Factor H (FH) and C4b-binding protein (C4BP) (FH/C4BP) transgenic mice) in a BALB/c background have been described previously (Ermert et al. 2015). FH/C4BP Tg mice express levels of FH and C4BP that are comparable to those found in human serum and show similar responses to a variety of stimuli as wild-type (wt) BALB/c mice (Ermert et al. 2015). Wild-type C57BL/6 mice were purchased from Jackson laboratories. Construction and characterization of C6 mice (C57BL/6 background) have been described previously (Ueda et al. 2019).

B. Mouse Vaginal Colonization Model of Gonorrhea

Female mice 6-8 weeks of age in the diestrus phase of the estrous cycle were started on treatment with 0.1 mg Premarin (Pfizer; conjugated estrogens) in 200 μL of water given s.c. on each of three days: −2, 0, and +2 (2 d before, the day of, and 2 d after inoculation) to prolong the estrus phase of the reproductive cycle and promote susceptibility to N. gonorrhoeae infection. Antibiotics (vancomycin and streptomycin) ineffective against N. gonorrhoeae were also used to reduce competitive microflora (Jerse et al. 2011). Mice were infected on day 0 with either strain H041 or FA1090 (inoculum specified for each experiment). Mice were intravaginally treated daily with 1 or 10 μg of the FH*/Fc fusion protein S2370 from day 0 until the conclusion of the experiment or were given a corresponding volume of PBS (vehicle controls).

C. Statistical Analysis

Concentration-dependent complement-mediated killing by FH*/Fc across strains was compared using 2-way ANOVA. Experiments that compared clearance of N. gonorrhoeae in independent groups of mice estimated and tested three characteristics of the data (Shaughnessy et al. 2016, Shaughnessy et al. 2018, Gulati et al. 2019): time to clearance, longitudinal trends in mean log₁₀ CFU, and the cumulative CFU as area under the curve (AUC). Statistical analyses were performed using mice that initially yielded bacterial colonies on days 1 and/or 2. Median time to clearance was estimated using Kaplan-Meier survival curves; times to clearance were compared between groups using the Mantel-Cox log-rank test. Mean log₁₀ CFU trends over time were compared between groups using 2-way ANOVA and Dunnett's multiple comparison test. The mean AUC (login CFU versus time) was computed for each mouse to estimate the bacterial burden over time (cumulative infection). The means under the curves of two groups were compared using the nonparametric Mann-Whitney test because distributions were skewed or kurtotic. The Kruskal-Wallis equality-of-populations rank test was also applied to compare more than two groups in an experiment.

D. Results

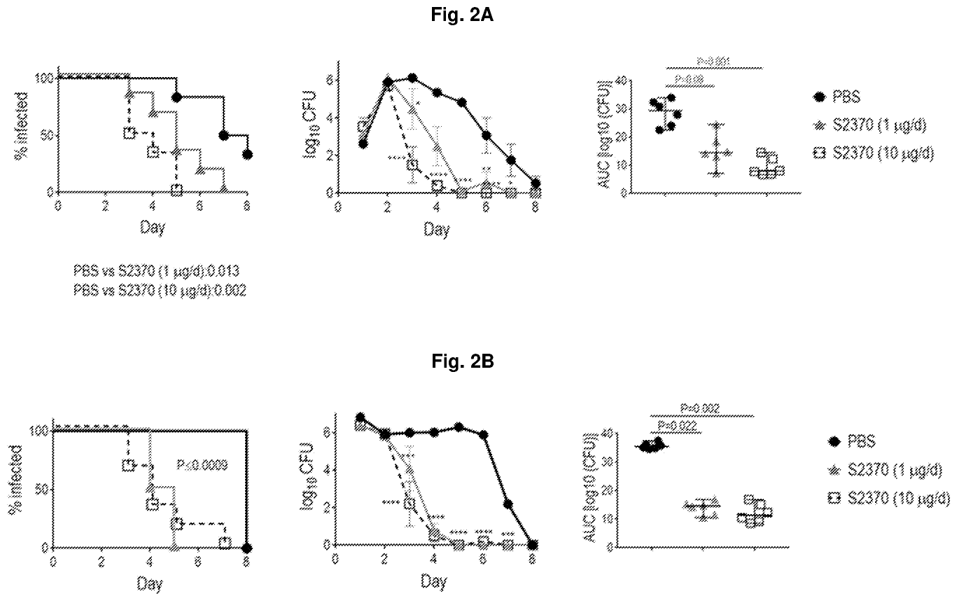

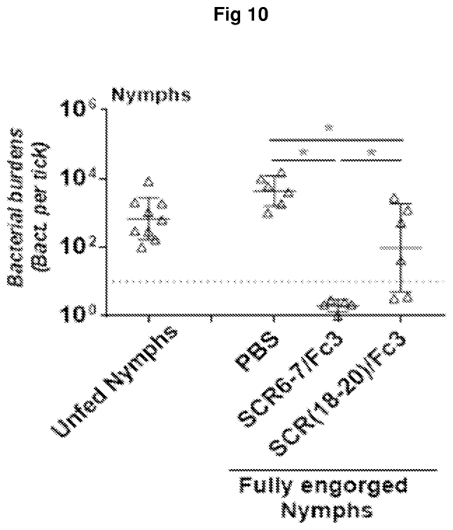

As shown by the results depicted in the plots of FIG. 2A and FIG. 2B, S2370 given daily intravaginally at doses of either 1 or 10 μg/d significantly attenuated both the duration and the burden of gonococcal vaginal colonization compared to vehicle control treated groups, when challenged with either 10⁶ CFU (FIG. 2A) or 10⁷ CFU (FIG. 2B) of bacteria. Overall, there were no significant differences in clearance between the 1 or 10 μg doses.

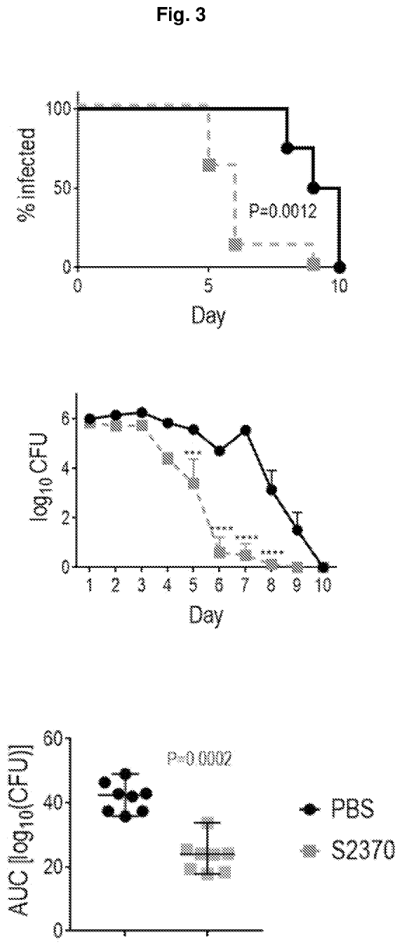

As shown by the results depicted in the plots of FIG. 3, S2370 was also efficacious against strain FA1090 in FH/C4BP transgenic mice when administered intravaginally at a dose of 10 μg/d.

### Example 9: Improvement in Process Yield without Impairment of Activity: Capping the N-Terminal Cys in FH*/Fc Improves Protein Yields and Retains Function

A distinctive feature of the FH*/Fc fusion proteins is the presence of an N-terminal cysteine. Proteins having such N-terminal cysteines are able to undergo a reaction called native chemical ligation, whereby the cysteine reacts with free thioester groups (Dawson et al. 1994, Gentle et al. 2004). Concentration and sterile filtration of all variants of FH*/Fc with N-terminal cysteine resulted in dramatic losses of protein; close to 50% versus the ˜20% loss seen with other plant-produced Fc fusions (Wycoff et al. 2011, Wycoff et al. 2015).

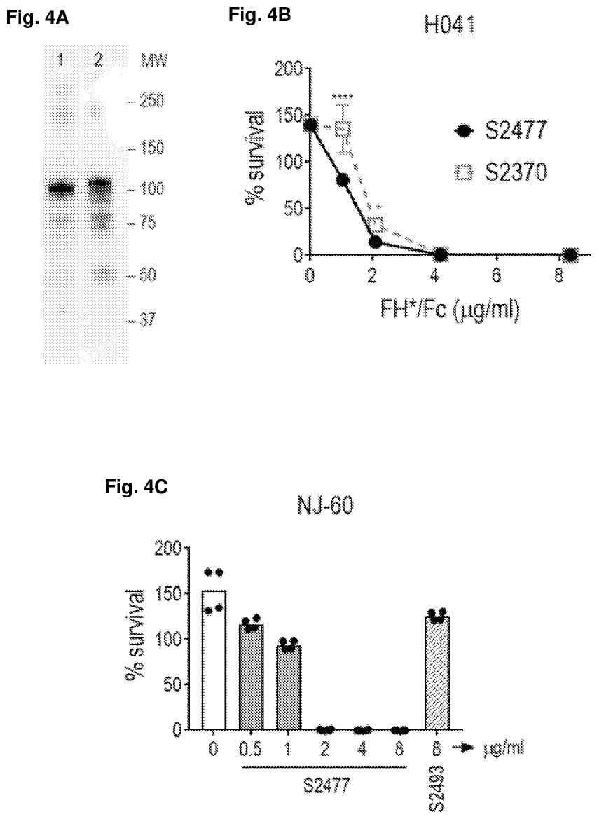

The native FH sequence includes two additional amino acids, TS, that are N-terminal to the cysteine. A FH*/Fc fusion protein (S2477) was designed, expressed, and purified with these two additional N-terminal amino acids (TS) capping the cysteine. As shown by the Western blot results depicted in FIG. 4A, S2477 showed fewer degradation products after purification compared to S2370. Thus, the capping of the N-terminal cysteine with the additional N-terminal TS amino acids overcame the previously noted loss during purification.

A comparison of the bactericidal activity of S2370 and S2477 against N. gonorrhoeae strain H041 was also carried out. As shown by the results plotted in FIG. 4B, S2477 exhibited slightly better activity than S2370 (BC₅₀ of 1.5 and 1.9 μg/ml respectively).

As shown by the results in FIG. 4C, the efficacy of S2477 against another ceftriaxone-resistant isolate, NJ60, was also confirmed (BC₅₀ of 1.5 μg/ml). By comparison, the S2493 fusion protein, which is a derivative of S2477 that includes D270A and K322A amino acid substitutions in Fc that abrogate C1q binding (Hezareh et al. 2001), was included as a negative control. S2493 showed no killing.

S2477 Requires an Intact Terminal Complement Pathway for Efficacy

C1q engagement by Fc is critical for the activity of CHO cell-produced FH*/Fc (Shaughnessy et al. 2018), suggesting that the classical complement pathway is required for efficacy of FH*/Fc. To determine whether complement alone acting through killing by membrane attack complex (MAC) insertion was necessary and sufficient for efficacy of FH*/Fc, we used C6^{-/-} mice (Ueda et al. 2019). C6 is the second step in the formation of the C5b-9 MAC pore. While C6^{-/-} mice lack the capacity to form MAC pores, they can generate C5a, which is important for chemotaxis of PMNs and opsonophagocytic killing of Neisseria (Densen et al. 1982, Konar et al. 2017).

Wild-type C57BL/6 control mice or C6^{-/-} mice (n=6/group) were infected with H041 and treated with either the S2477 or S2493 fusion protein (each given at 5 μg intravaginally daily, starting on day 0, through day 7) or PBS vehicle control.

Although S2477 was efficacious in WT C57BL/6 mice, all efficacy was lost in C6^{-/-} mice. FH*/Fc that lacked the ability to activate complement (S2493) was inactive in both C6^{-/-} and wild-type mice. Taken together, these data show that complement alone is necessary and sufficient for efficacy of FH*/Fc in the mouse vaginal colonization model of gonorrhea.

### Example 10: FH*/Fc Fusion Proteins Kill Multiple Strains/Species of Lyme Borreliae In Vitro

This example illustrates a study of the ability of FH*/Fc fusion proteins of the present disclosure to kill Lyme borreliae in vitro.

The plant-made fusion proteins, FH*/Fc1 (S2477, p1394, pTRAk-c-Iph-(TS)FH*-(GGGGS)₂-hFc1) (also referred to herein as “SCR18-20-Fc1”), and FH(6-7)/Fc1 (S2417, p1365, pTRAk-c-Iph-FH(6-7)-(GGGGS)₃-hFc1) (also referred to herein as “SCR6-7/Fc1”) that were previously shown to kill N. gonorrhoeae were tested for their ability to facilitate in vitro complement-mediated killing of Borrelia burgdorferi (Bb) strains B31-5A4 and 297, as well as Borrelia afzelii (Ba) strain VS461. These two species (members of the Lyme borreliae) are the main causal agents of Lyme disease in the US and European Union, respectively.

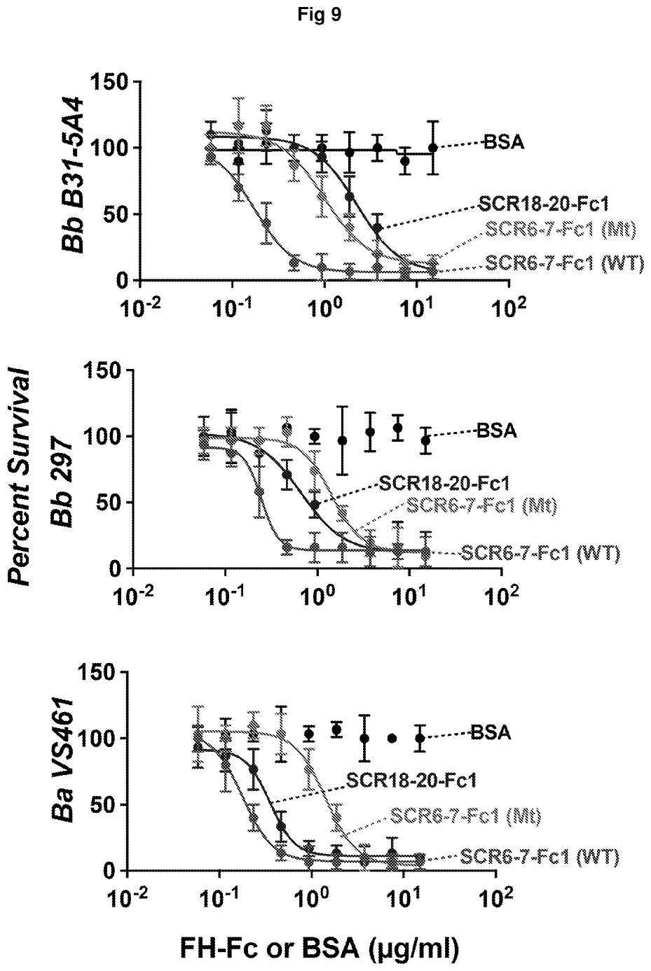

As shown by the results shown in FIG. 9 and summarized in Table 4, the fusion proteins exhibited efficacy against the Bb and Ba strains with EC₅₀ ranging from 0.16 μg/mL to 0.36 μg/mL.

TABLE 4
EC50 (μg/mL) of FH/Fc variants against Lyme borreliae
in complement-dependent bactericidal assay.

| Construct | Ba VS461 | Bbav Pbi | Bg ZQ1 |
|---|---|---|---|
| SCR(6-7)/Fc1 | 0.17 | ND | ND |
| SCR(6-7)/Fc3 | 0.16 | 11.24 | NK |
| SCR(6-7)/Fc1 (MT) | 1.47 | ND | ND |
| FH*/Fc1 | 0.36 | ND | ND |
| FH*/Fc3 | 0.23 | 6.00 | 6.85 |
| FH*/Fc1 (MT) | NK | NK | NK |

The bacterial killing assays were performed as in FIG. 2.
NK, not killed at 30 μg/ml of FH/Fc;
ND, assay not done

Further, the potency of SCR(6-7)/Fc1 (mt), a variant of SCR(6-7)/Fc1 containing two Fc mutations that eliminate C1q binding (D270A/K322A) (Idusogie et al. 2000, Idusogie et al. 2001), was reduced ˜7-fold (FIG. 9). Both Fc-dependent and Fc-independent activities of FH(6-7)/Fc1 are involved in killing Lyme borreliae. In the in vitro potency assay results in Table 4 and FIG. 9, the FH-Fc fusions or BSA (negative control) were mixed at indicated dilutions with 40% human serum (as complement source) and B. burgdorferi strains B31-5A4 and 297 or B. afzelii strain VS461. Surviving spirochetes were quantified using dark-field microscopy after 24 hr. Percent survival was derived from the proportion of FH-Fc-treated to untreated spirochetes. Data shown in FIG. 9 are the mean±SD of percent survival derived from three microscope fields for each sample. Table 4 shows the EC50 (concentration of FH/Fc that results in 50% killing) of each variant.

To improve the Fc-dependent borreliacidal activity of both the FH*/Fc and the FH(6-7)/Fc fusion proteins, the IgG1 Fc was replaced with Fc from IgG3 while retaining the shortened hinge of IgG1 described above to enhance the binding of the Fc to C1q and increase complement activation (Natsume et al. 2008, Stapleton et al. 2011, Giuntini et al. 2012, Giuntini et al. 2016). The IgG3 allotype used (Martensson et al. 1966, Steinberg 1969) has a half-life comparable to IgG1 (3 weeks) in humans while retaining IgG3's superior CDC activity (Stapleton et al. 2011). While the efficiency of FH*/Fc IgG3-mediated killing of N. gonorrhoeae increased 10-fold or more (data not shown), FH(6-7)/Fc3 (S2635, p1475 N-terminal FH(6-7)-(GGGGS)₂-hFc3(IgG1 hinge)(435H)) is only 10% more potent than FH(6-7)-(GGGGS)₂-Fc1, whereas FH*/Fc3 (S2499, p1407, N-terminal TS, FH*-(GGGGS)₂ linker hFc3 with IgG1 hinge) displayed 36% more robust killing than FH*-Fc1(S2368, p1399 FH*-(GGGGS)₂ linker hFc1) (Table 4).

The FH-Fc3(IgG1 hinge) constructs were tested and shown to be effective on Bbav and Bg. While both FH*/Fc3 (IgG1 hinge) and FH(6-7)/Fc3(IgG1 hinge) killed Bbav, with EC₅₀ of greater than 6 μg/mL (Table 4), FH*/Fc3(IgG1 hinge) but not FH(6-7)Fc3(IgG1 hinge) eradicated Bg (Table 4, EC₅₀=6.85 μg/ml).

The FH/Fc fusion protein constructs of the present disclosure thus kill multiple strains/species of Lyme borreliae in vitro, but the efficacious constructs vary for the eradication of different Lyme borreliae species.

### Example 11: FH (6-7)/Fc3 Prevents LD Infection by Blocking Bb Survival in Fed Ticks and Tick-to-Host Transmission

This example illustrates a study to determine whether the fusion protein FH(6-7)/Fc3 (S2635, P1475 pTRAk-c-Iph-FH(6-7)-(GGGGS)₂-hFc3(IgG1 hinge)(435H), or the fusion protein FH*/Fc3 (S2634 p1425 pTRAk-c-Iph-hFc3(IgG1 hinge)(435H)-(GGGGS)₂-(TS)FH(D18-20) can prevent Lyme borreliae infection in vivo.

C3H/HeN mice were subcutaneously administered 2 mg/kg or 20 mg/kg of SCR(6-7)/Fc3 or FH*/Fc3 fusion protein (or PBS; negative control) one day prior to being fed on by I. scapularis nymphal ticks (nymphs) carrying Bb strain B31-5A4, which were generated as described (Hart et al. 2018). The nymphs fed until fully engorged (4 days post feeding, 4 dpf). Bb burdens (determined by qPCR) in all nymphs feeding on FH(6-7)/Fc3-treated mice were lower than the detection limit (10 bacteria per tick) (FIG. 10). The geometric mean values of bacterial burdens in all nymphs feeding on FH*/Fc3-treated mice remained greater than the detection limit, but still significantly lower than the nymphs feeding on negative control PBS-treated mice (FIG. 10). Thus, FH(6-7)/Fc3 and FH*/Fc3 treatment completely and partially eradicated respectively Bb in fed nymphs.

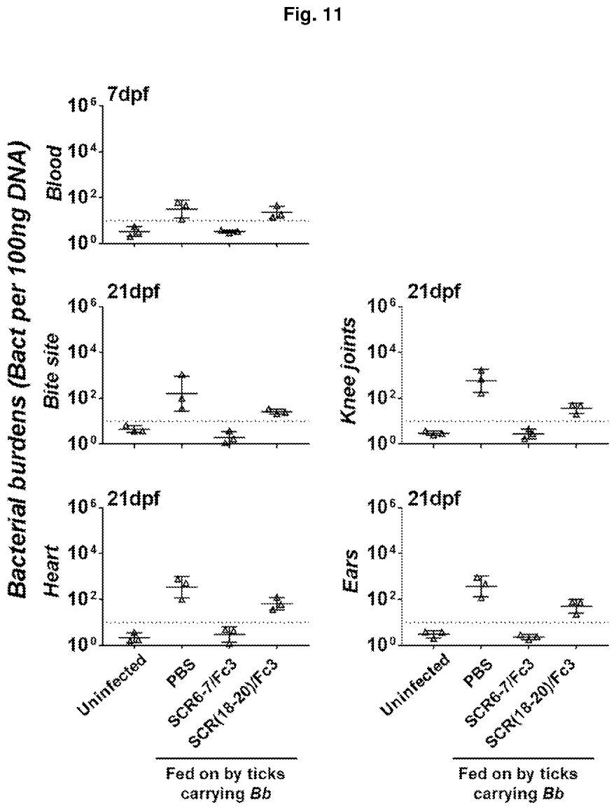

Blood and tissues were collected from the treated and control C3H/HeN mice at 7 and 21 dpf, respectively. Bacterial burdens in samples from FH(6-7)/Fc3-treated mice fed on by Bb-carrying ticks were below detection limits (10 bacteria per 100 μg DNA), similar to samples from mice not fed on by ticks (FIG. 11). However, the bacterial burdens from FH*/Fc3-treated mice were all above detection limits and indistinguishable from PBS-treated mice (FIG. 11). Antibodies against a Bb antigen, designated C6, which is commonly used in evaluating seropositivity in human LD (Liang et al. 1999) were assayed. All FH (6-7)/Fc3-treated mice were seronegative whereas all FH*/Fc3-treated or PBS-treated mice were seropositive (data not shown).

These results indicate that FH(6-7)/Fc3 is capable of preventing LD infection by Bb and Ba, in mice by blocking tick-to-host transmission and correlates with its ability to eradicate Bb in feeding nymphs.

### Example 12: Improvements to FH*-Linker-Fc

Example 11 demonstrated that FH(6-7)/Fc3 alone is sufficient as a PrEP for Bb-associated LD infection. These in vitro results show that the efficacious FH/Fc constructs vary in their ability to eradicate different Lyme borreliae species. To achieve the broadest possible cross protection against LD caused by multiple Lyme borreliae species (or even other TBPs), both FH(6-7)/Fc3 and FH*/Fc3 may be used simultaneously.

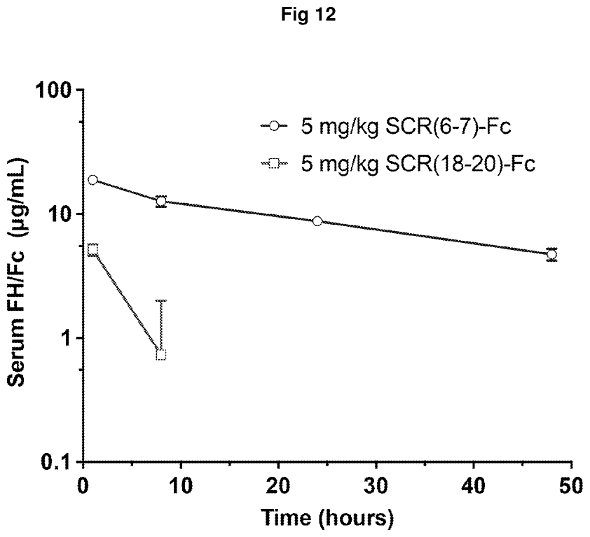

One reason for the lower in vivo efficacy of FH*/Fc3 may be its rapid clearance from circulation (see e.g., pharmacokinetic data plotted in FIG. 12). The following examples produce FH*/Fc3 variants incorporating protein sequence modifications that:
i) enhance opsonophagocytosis of Lyme borreliae,
ii) reduce off-target interactions with host cell surface molecules and
iii) extend the half-life of both FH(6-7)/Fc3 and FH*/Fc3 proteins. Each or all of these modifications may result in a superior product for PrEP.

12A: Variants of FH/Fc3 Fusion Protein Using Long Hinge of IgG3

In the foregoing exemplary FH/Fc3 fusions, the Fc sequence used includes the truncated IgG1 hinge. A new variant of FH(6-7)/Fc3 was prepared that replaces the short IgG1 hinge in the current construct with a longer IgG3 hinge. While engineered antibodies with the shorter IgG1 hinge are more potent in complement activation than wild-type IgG3, longer IgG3 hinges confer significantly greater opsonophagocytic activity (but less complement-dependent killing). A short IgG1 hinge FH(6-7)/Fc3 will be prepared and compared to a longer IgG3 hinge (LH) variant for increased in vivo potency.

Variant 1: FH(6-7)/Fc3-LH (pTRAk-c-Iph-FH(6-7)-(GGGGS)₂-hFc3(IgG3 hinge)(435H)

In this variant the human Fc3 replaces the truncated IgG1 hinge sequence with an IgG3 hinge. The IgG3 hinge has the amino acid sequence of SEQ ID NO: 23:

(SEQ ID NO: 23)
N2-terminal-
ELKTPLGDTTHTCPRCPEPKSCDTPPPCPRCPEPKSC
DTPPPCPRCPEPKSCDTPPPCPRCP-C terminal

Variant 2: SCR(19-20)/Fc3-LH

In the in vivo assay FH*/Fc3(IgG1 hinge) is clearly less effective (FIG. 10 and FIG. 11) and its shorter in vivo half-life is likely the major contributing factor to this discrepancy (FIG. 12). This short half-life of FH*/Fc3 caused by its clearance, when administered to a subject, by the asialoglycoprotein receptor or the mannose receptor due to the presence of an N-glycan in SCR18, exposed on the exterior of the protein. Eliminating SCR18 (and the attached N-glycan) is expected to improve the half-life of the molecule.

SCR(19-20)/Fc3-LH may be produced by modification of plasmid p1425 (SEQ ID NO: 17; FIG. 21) using primer mutagenesis or de novo synthesis.

Primer Mutagenesis:

The DNA encoding FH domains 19-20 was amplified from plasmid p1425 (p1425 pTRAk-c-Iph-hFc3(IgG1 hinge)(435H)-(GGGGS)₂-(TS)FH(D18-20)) using the forward primer 5′-GAA AGC GGC CGC GGG CGG CGG TGG TTC TGG TGG TGG CGG GAG CGA CTC CAC CGG TAA GTG TGG-3′ (SEQ ID NO: 24), and the reverse primer 5′-TTT CTC TAG ATT ACC TCT TGG CAC AGG TGG-3′ (SEQ ID NO: 25) for FH domain 20 of the FH19-20 domains. After PCR amplification, the amplified fragment encoding SCR 19-20 was digested with NotI and XbaI and cloned into the corresponding restriction sites in p1425 (pTRAk-c-Iph-hFc3(IgG1 hinge)(435H)-(GGGGS)₂-(TS)FH(D18-20)) plasmid. The resulting plasmid was verified by DNA sequencing and used to transiently transform N. benthamiana leaves. Leaves were collected after 7 days, and FH/Fc was purified by passage over protein A—agarose. Protein concentrations were determined using the BCA protein Assay kit (Pierce); mass was determined by Coomassie Blue staining of proteins separated by SDS-PAGE.

De Novo Synthesis:

The DNA encoding FH domains 19-20 having the same optimized sequence as SEQ ID NO: 15 cloned in p1425 and the GS2 linker were synthesized by GeneWiz to incorporate terminal NotI and XbaI restriction sites. The synthesized fragments were digested with NotI and XbaI and cloned into the corresponding restriction sites of p1425 (pTRAk-c-Iph-hFc3(IgG1 hinge)(435H)-(GGGGS)₂-(TS)FH(D18-20)) plasmid. The resulting plasmids was verified by DNA sequencing and used to transiently transform N. benthamiana leaves. Leaves were collected after 7 days, and FH/Fc was purified by passage over protein A—agarose. Protein concentrations were determined using the BCA protein Assay kit (Pierce); mass was determined by Coomassie Blue staining of proteins separated by SDS-PAGE.

Variant 3: SCR 20/Fc3-LH

FH binds to Bb OspE through three amino acids on SCR20, R1182, E1195 and R1215 (known as the “common microbial binding site” (Meri et al. 2013, Kolodziejczyk et al. 2017)). In an alternative form SCR18 and SCR19 may be removed from the construct retaining SCR20. SCR(20)/Fc3-LH may be produced by modification of plasmid p1425 (SEQ ID NO: 17) using primer mutagenesis or de novo synthesis.

Primer Mutagenesis:

The DNA encoding FH domains 20 was amplified from a plasmid p1425 (p1425 pTRAk-c-Iph-hFc3(IgG1 hinge)(435H)-(GGGGS)₂-(TS)FH(D18-20)) using the forward primer 5′-GAA AGC GGC CGC GGG CGG CGG TGG TTC TGG TGG TGG CGG GAG CCA TCC CTG CGT GAT CAG CCG-3′ (SEQ ID NO: 26), and the reverse primer 5′-TTT CTC TAG ATT ACC TCT TGG CAC AGG TGG-3′ (SEQ ID NO: 27) for the FH 20 domain. After PCR amplification the amplified fragment encoding SCR 20 was digested with NotI and XbaI and cloned into the corresponding restriction sites in p1425 (pTRAk-c-Iph-hFc3(IgG1 hinge)(435H)-(GGGGS)₂-(TS)FH(D18-20)) plasmid. The resulting plasmid was verified by DNA sequencing and used to transiently transform N. benthamiana leaves. Leaves were collected after 7 days, and SCR20/Fc was purified by passage over protein A—agarose. Protein concentrations were determined using the BCA protein Assay kit (Pierce); mass was determined by Coomassie Blue staining of proteins separated by SDS-PAGE.

De Novo Synthesis:

The DNA encoding FH domain 20 (SCR20), having the same optimized sequence as the corresponding sequence in p1425 (sequence ID 6) and the GS2 linker were synthesized by GeneWiz to incorporate terminal NotI and XbaI restriction sites. The synthesized fragments were digested with NotI and XbaI and cloned into the corresponding restriction sites of p1425 (pTRAk-c-Iph-hFc3(IgG1 hinge)(435H)-(GGGGS)₂-(TS)FH(D18-20)) plasmid. The resulting plasmids plasmid was verified by DNA sequencing and used to transiently transform N. benthamiana leaves. Leaves were collected after 7 days, and SCR20/Fc was purified by passage over protein A—agarose. Protein concentrations were determined using the BCA protein Assay kit (Pierce); mass was determined by Coomassie Blue staining of proteins separated by SDS-PAGE.

12B: Modification of SCR 20 Variants to Reduce Off Target Effects on Host Cells and Tissues

The SCR20-containing variant produced in section 12A that retains the same (or close to the same) potency as FH*/Fc3 is used to generate two additional variants to address the possibility that the short half-life of FH*/Fc's is due to its binding to endothelial cell surfaces. A normal function of FH is to bind simultaneously to C3 fragments deposited on host cells and to cell-surface glycosaminoglycans through domains 19 and 20, respectively, thereby limiting complement activation targeting host cells. (Kajander et al. 2011, Blaum et al. 2015, Wong et al. 2016). Since FH*/Fc can compete with FH to bind simultaneously to C3 fragments deposited on host cells and to cell-surface glycosaminoglycans through domains 19 and 20, such competitive displacement of FH may cause activation of complement on and damage to host cells.

The FH*/Fc3 fusion protein variants described elsewhere herein include a mutation in SCR19 (D to G at position 1119) (Ferreira et al. 2009, de Cordoba et al. 2012), which abrogates its ability to bind to C3b-coated host surfaces, but does not affect its ability to bind to and kill bacterial targets such as Neisseria gonorrhoeae (Shaughnessy et al. 2016). However, constructs comprising SCR 20, e.g., FH*/Fc3, SCR(19-20)/Fc3, and SCR(20)/Fc3, retain the ability to bind to heparin/heparan sulfate-containing surfaces (Schmidt et al. 2008) and endothelial cells (Manuelian et al. 2003). Three amino acids (R1203, R1206 and R1210) in SCR20 are critical for this binding activity and specific mutations (R1203E, R1206E and R1210S) eliminated that binding (Jokiranta et al. 2005). Additionally, Factor H related proteins FHR-3 and FHR-4, which are sequentially similar to human FH do not bind to heparin, because the amino acids equivalent to R1203, R1206, and R1210 are replaced by leucine, asparagine and serine, respectively (Hellwage et al. 2002).

Two new variants are provided with the above-mentioned mutated amino acids in R1203, R1206, and R1210 of SCR20 to eliminate the off-targeting heparin/endothelial cell-binding activity. The modification of R1203, R1206 and R1210 is carried out by site directed mutagenesis of the nucleic acid sequence encoding the residues at these positions, using overlapping PCR primers in the following non limiting example. The same results can be obtained by de novo synthesis of the nucleotide sequences encoding the desired residues and ligating them into the proper position. Alternatively, these alterations may be accomplished by gene editing using Crispr Cas9.

Variant 4: SCR(18-20)/Fc3, SCR(19-20)/Fc3, or SCR20/Fc3 with R1203 E/R1206E/R1210S Mutations

Starting with p1425 or the variant 2 or variant 3 produced in 12A above, the DNA encoding SCR 20 was modified by site-directed mutagenesis using the forward primer 5′-GAG GGT TAC GAG CTC TCC TCC TCC TCC CAT ACC CTC AGG ACC ACC-3′ (SEQ ID NO: 28), and the reverse primer 5′-GGA GGA GGA GAG CTC GTA ACC CTC CTT GCA CAC AAA CTC GAC GC-3′ (SEQ ID NO: 29) to introduce three mutations (R1203E, R1206E, and R1210S) in SCR 20. After PCR amplification the amplified fragment encoding the modified SCR 20 was digested with NotI and XbaI and cloned into the corresponding restriction sites in p1425 (pTRAk-c-Iph-hFc3(IgG1 hinge)(435H)-(GGGGS)₂-(TS)FH(D18-20)) plasmid. The resulting plasmid was verified by DNA sequencing and used to transiently transform N. benthamiana leaves. Leaves were collected after 7 days, and modified SCR20/Fc was purified by passage over protein A—agarose. Protein concentrations were determined using the BCA protein Assay kit (Pierce); mass was determined by Coomassie Blue staining of proteins separated by SDS-PAGE.

Variant 5 (S2538); SCR(18-20)/Fc3, SCR(19-20)/Fc3 or SCR20/Fc3 with R1203UR1206N/R1210S Mutations.

The DNA encoding FH domain 20 was amplified from a plasmid p1425 (p1425 pTRAk-c-IphhFc3(IgG1 hinge)(435H)-(GGGGS)2-(TS)FH(D18-20)) FH SCR 20 was modified by site-directed mutagenesis using the forward primer 5′-CTC GGT TAC AAC CTC TCC TCC TCC TCC CAT ACC CTC AGG ACC ACC-3′ (SEQ ID NO: 30), and the reverse primer 5′-GGAGGAGGA GAG GTT GTA ACC GAG CTT GCA CAC AAA CTC GAC GC-3′ (SEQ ID NO: 31) to introduce three mutations (R1203L/R1206N/R1210S) in SCR 20. After PCR amplification the amplified fragment encoding SCR 20 was digested with NotI and XbaI and cloned into the corresponding restriction sites in p1425 (pTRAk-c-Iph-hFc3(IgG1 hinge)(435H)-(GGGGS)₂(TS)FH(D18-20)) plasmid. The resulting plasmid was verified by DNA sequencing and used to transiently transform N. benthamiana leaves. Leaves were collected after 7 days, and modified SCR20/Fc was purified by passage over protein A-agarose. Protein concentrations were determined using the BCA protein Assay kit (Pierce); mass was determined by Coomassie Blue staining of proteins separated by SDS-PAGE.

Because binding of FH to B. burgdorferi OspE occurs through contact points at R1182, E1195 and R1215 (Kolodziejczyk et al. 2017), which lie on the opposite side of SCR20 from the heparin/glycosaminoglycan binding site (see structure, PBDe 5nbq: https://www.ebi.ac.uk/pdbe/entry/pdb/5nbq), the above-described Variant 4 and Variant 5 fusion proteins are unlikely to affect OspE binding and thus, will not negatively impact killing of Lyme borreliae.

### Example 13: Fc Mutations that Enhance Half-Life

13A: Introduction of triple mutation M252Y/S254-1/1-256E into Fc of IgG3

The half-life of the above-described constructs can be increased by introducing a triple mutation, M252Y/S254-1/1-256E (YTE), into the Fc of IgG3 (Dall'Acqua et al. 2006, Robbie et al. 2013, Yu et al. 2017). Starting with p1425 or a variant produced in Example 12, the polynucleotide sequence encoding Fc3 is modified by site-directed mutagenesis using the forward primer, 5′-ACT CTT TAC ATT ACC AGG GAG CCT GAA GTT ACT TGC GTT GTT-3′ (SEQ ID NO: 32), and the reverse primer, 5′-AGG CTC CCT GGT AAT GTA AAG AGT GTC CTT TGG CTT AGG-3′ (SEQ ID NO: 33) to introduce 3 mutations (M252Y, 5254T, and T256E) in the Fc sequence. After PCR amplification the amplified fragment encoding the modified Fc sequence was digested with NotI and XbaI and cloned into the corresponding restriction sites in p1425 (pTRAk-c-Iph-hFc3(IgG1 hinge)(435H)-(GGGGS)2-(TS)FH(D18-20)) plasmid. The resulting plasmid was verified by DNA sequencing and used to transiently transform N. benthamiana leaves. Leaves were collected after 7 days, and SCR20/Fc was purified by passage over protein A-agarose. Protein concentrations were determined using the BCA protein Assay kit (Pierce); mass was determined by Coomassie Blue staining of proteins separated by SDS-PAGE.

The same results can be obtained by de novo synthesis of the nucleotide sequences encoding the desired residues and ligating them into the proper position.

13B: Fc LS Mutations to Enhance Half-Life

The half-life of the above-described constructs can be increased by introducing a double mutation, M428L/N434S (LS), in the Fc of the FH-Fc fusion protein(s). Starting with p1425 or a variant as produced in Example 12, the DNA encoding Fc is modified by site-directed mutagenesis using the forward primer, 5′-TCT GTT CTT CAT GAA GCA TTA CAT TCT CAC TTC ACT CAA AAG TCT CTT-3′ (SEQ ID NO: 34), and the reverse primer, 5′-GTG AGA ATG TAA TGC TTC ATG AAG AAC AGA GCA ACT GAA AAT ATT-3′ (SEQ ID NO: 35) to introduce 2 mutations (M428L and N4345) into the Fc sequence. After PCR amplification the amplified fragment encoding the modified Fc sequence was digested with NotI and XbaI and cloned into the corresponding restriction sites in p1425. The resulting plasmid is verified by DNA sequencing and used to transiently transform N. benthamiana leaves. Leaves are collected after 7 days, and SCR20/Fc is purified by passage over protein A—agarose. Protein concentrations are determined using the BCA protein Assay kit (Pierce); mass is determined by Coomassie Blue staining of proteins separated by SDS-PAGE.

The same results can be obtained by de novo synthesis of the nucleotide sequences encoding the desired residues and ligating them into the proper position. Additionally, these alterations may be made by gene editing using CRISPR Cas9.

13C: Substitution of Fc of IgG3 Allele IGHG3*17 for Fc of IgG3

The Fc of IgG3 allele IGHG3*17, also called G₃m(s*) (IMGT accession number: AJ390272), which naturally has a H (histidine) at position 435 (Eu numbering), confers a longer half-life and allows purification by Protein A and may be used instead of the Fc of IgG3 described in the foregoing examples. The Fc amino acid sequence of this IGHG3*17 (excluding the hinge) is:

(SEQ ID NO: 36)
APELLGGPSVFLFPPKPKDTLMISRTPEVTCVVVD
VSHEDPEVQFKWYVDGVEVHNAKTKPREEQYNSTF
RVVSVLTVLHQDWLNGKEYKCKVSNKALPAPIEKT
ISKTKGQPREPQVYTLPPSREEMTKNQVSLTCLVK
GFYPSDIAMEWESSGQPENNYKTTPPVLDSDGSFF
LYSKLTVDKSRWQQGNIFSCSVMHEALHNHYTQKS
LSLSPGK.

13C.1: Cloning IGHG3*17 into p1425 in Lieu of Fc of IgG3

A DNA sequence that includes the sequence ctgcaggtgttcactcc (SEQ ID NO: 37) (incorporating a Pst I restriction site and the last few amino acids of the signal peptide) followed by a sequence encoding either the IgG1 hinge or the IgG3 hinge (see definitions), followed by a sequence encoding the IGHG3*17 allele amino acid sequence, followed by a Not I restriction site was synthesized by GeneWiz. The synthesized fragments are digested with PstI and NotI and cloned into the p1425 (pTRAk-c-Iph-hFc3(IgG1 hinge)(435H)-(GGGGS)2-(TS)FH(D18-20)) plasmid also digested with PstI and NotI. The encoded protein has Fc of IgG3 allele IGHG3*17 at the N-terminal end and SCR(D18-20) at the C-terminal end. The resulting plasmid is verified by DNA sequencing and used to transiently transform N. benthamiana leaves. Leaves are collected after 7 days, and the protein produced was purified by passage over protein A-agarose. Protein concentrations are determined using the BCA protein Assay kit (Pierce); mass is determined by Coomassie Blue staining of proteins separated by SDS-PAGE. This new variant is designated Fc3(*17)/SCR(D18-20).

13C.2: Cloning IGHG3*17 into p1475 in Lieu of Fc of IgG3 or Fc of IgG1 Downstream of SCR(6-7)

A DNA sequence that incorporates a SacI restriction site (GAGCTCT) followed by a sequence encoding either the IgG1 hinge or the IgG3 hinge (see definitions), followed by a sequence encoding the IGHG3*17 allele amino acid sequence, followed by a stop codon and the Xba I restriction site (AGATCT) was synthesized by GeneWiz. The synthesized fragments were digested with SacI and XbaI and cloned into plasmid p1475 (pTRAk-c-Iph-FH(6-7)-(GGGGS)2-hFc3(IgG1 hinge)(435H)) also digested with SacI and XbaI. The encoded protein has SCR(6-7) at the N-terminal end and Fc of IgG3 allele IGHG3*17Fc3 at the C-terminal end. The resulting plasmid is verified by DNA sequencing and used to transiently transform N. benthamiana leaves. Leaves are collected after 7 days, and FH/Fc is purified by passage over protein A—agarose. Protein concentrations are determined using the BCA protein Assay kit (Pierce); mass is determined by Coomassie Blue staining of proteins separated by SDS-PAGE. This new variant is designated SCR(6-7)/Fc3(*17).

### Example 14: Complement Activated Killing of Methicillin-Resistant Staphylococcus aureus (MRSA) Using Various Concentrations and Configurations of FH*/Fc, Fusion Proteins PMNs and Normal Human Serum

To determine the effect of FH*/Fc fusion proteins on the survival of methicillin resistant Staphylococcus aureus bacteria strain R7 when challenged with PMNs, mid-log R7 (2×10⁷ cfu)+/−fusion protein at increasing concentrations ranging from 4.5 to 9 μg/mL were incubated with 2.5, 5 or 10% normal human serum (NHS) in 500 μL total volume at 37 C. After 15 minutes, to allow for complement mediated opsonization of bacteria, PMNs were added at a ratio of 1:10 (PMN to bacteria). Samples were then rotated for 75 minutes at 37 C to permit phagocytosis. Mid-log R7 challenged with serum and PMNs (without fusion protein) were used as controls and represented 100% survival of bacteria for all assays. Following the incubation with PMNs, samples were serially diluted then plated onto Columbia 2% NaCl plates (at least two plates per sample) and incubated overnight. The following day, colonies were counted as a measure of bacterial survival. Percent survival was calculated by comparing colony counts from fusion-protein treated samples to control plates.

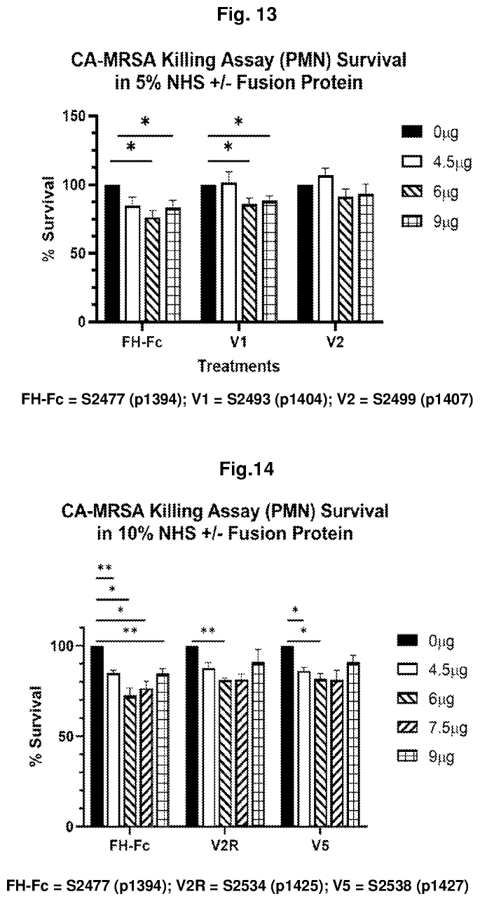

For 2.5% NHS, FH*/Fc showed the greatest reduction in MRSA survival (17.5% reduction), however, this result was not significant (data not shown). As shown by the results plotted in FIG. 13, in 5% NHS, both FH*/Fc fusion S2477 (p1394) and the Variant 1 fusion, S2493 (p1404) reduced survival of MRSA R7 compared to control. Although the reduction was slight (14-21% reduction in survival), these results were statistically significant (p 0.01).

As shown by the results plotted in FIG. 14, in 10% NHS, the FH*/Fc fusion protein, S2477 (p1394) showed the greatest reduction in survival (6 μg FH*/Fc, 27.4% reduction in survival) compared to control followed by Variant 2R fusion, S2534 (p1425) and Variant 5 fusion, S2538 (p1427) at the same concentration of fusion protein (one-way ANOVA, mixed-effects analysis, *, p<0.03,**, p<0.01).

### Example 15: pH Effects on Binding of FH*-Fc and FH 6,7-Fc to N. gonorrhoeae in Relationship to the pH of the Female Genital-Reproductive Tract

There is a pronounced pH gradient within the female genital-reproductive tract. This gradient is not disrupted in women with an abnormal vaginal microbiota. The pH gradient in the lower reproductive canal is most acidic in the lower vagina and most alkaline in the upper uterine cavity. Women with an abnormal vaginal microbiota have an increased pH in the lower vagina compared to the other groups. Among nonpregnant women with normal vaginal microbiome, there is a striking pH gradient with a median value of 3.9 (range: pH 3.6-4.3) in the lower vagina, 5.7 (5.2-6.3) in the upper vagina, a small but significant gradient within the cervical canal, and not less than 7.7 (7.5-7.8) in the upper uterine cavity. In early pregnancy and at-term pregnancy, the values in the vagina were rather close to those from nonpregnant women; however, for at-term pregnancy, the values within the cervical canal were decreased by about 1.0 pH (Lykke et al. 2021).

This example illustrates a study of the ability of the FH*/Fc fusion protein, S2534, and the FH 6,7/Fc fusion protein, S2635, to bind to Ng H401 was compared at 6 different pHs between 3.1 and 8.1

Materials and methods: The pH dependent assays for binding to Ng H401 were carried out as follows. 1) Coat 96 well plastic plate w/FH18-20-hIgA2 (PBI, #S2585) at 5 mcg/ml in 1×PBS, 50 mcl/well, 60 min, 37° C. 2) Wash w/1×PBS after each step prior to OPD development. 3) Block w/5% non-fat dry milk in 1×PBS (MOOP), 100 mcl/well, 15 min, 37° C. 4) Attach Ng H041 (paraformaldehyde fixed) (UMass) at OD600=0.05 in 1×PBS, 50 mcl/well, 60 min, 37° C. 5) Bind samples of hFc3-(G₄S2)-(TS)FH* (S2534) or FH(6-7)-(G₄S2)-hFc3 (S2635) (3× series starting at 10 mcg/ml) in 10 mM Glycine, 10 mM Acetate, 10 mM Citrate, 10 mM Histidine, 10 mM Phosphate, 100 mM NaCl, 10 mM Tris, pH'ed as indicated, 50 mcl/well, 60 min, 37° C. 6) Detect w/goat anti-huIgG, Fc (mouse absorbed)-HRP (Jackson) at 0.5 mcg/ml in MOOP, 50 mcl/well, 60 min, 37° C. 7) Develop with OPD/Citrate, 50 mcl/well, at room temp, 10 min. 8) Stop w/1 N H2SO4, 50 mcl/well, at room temp. 9) Read at 490 nm via a Synergy™ HT Multi-Detection Microplate Reader (BioTek Instruments).

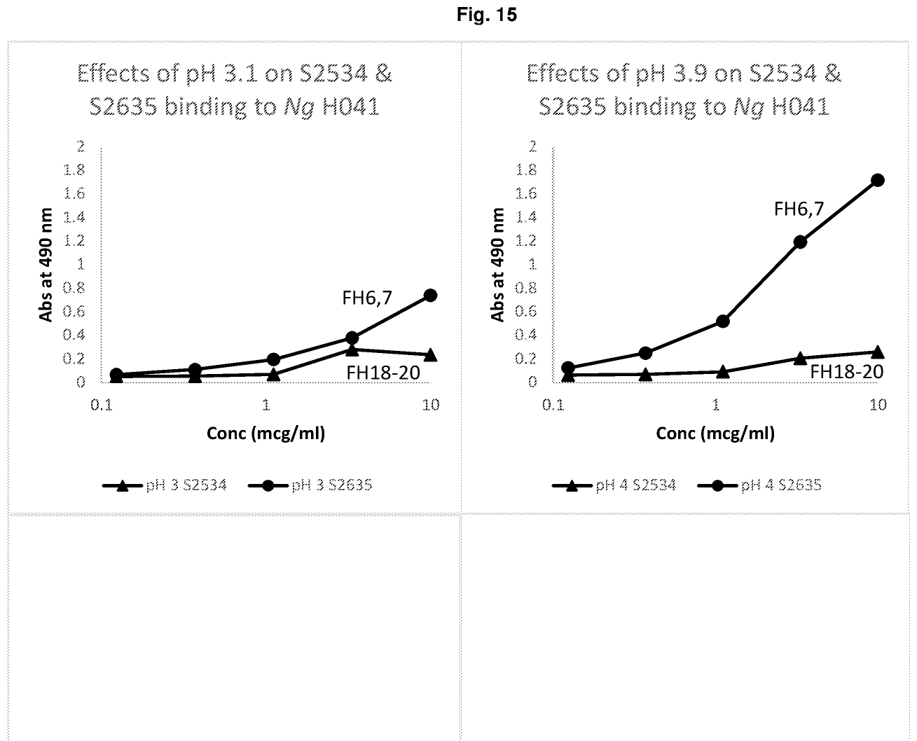

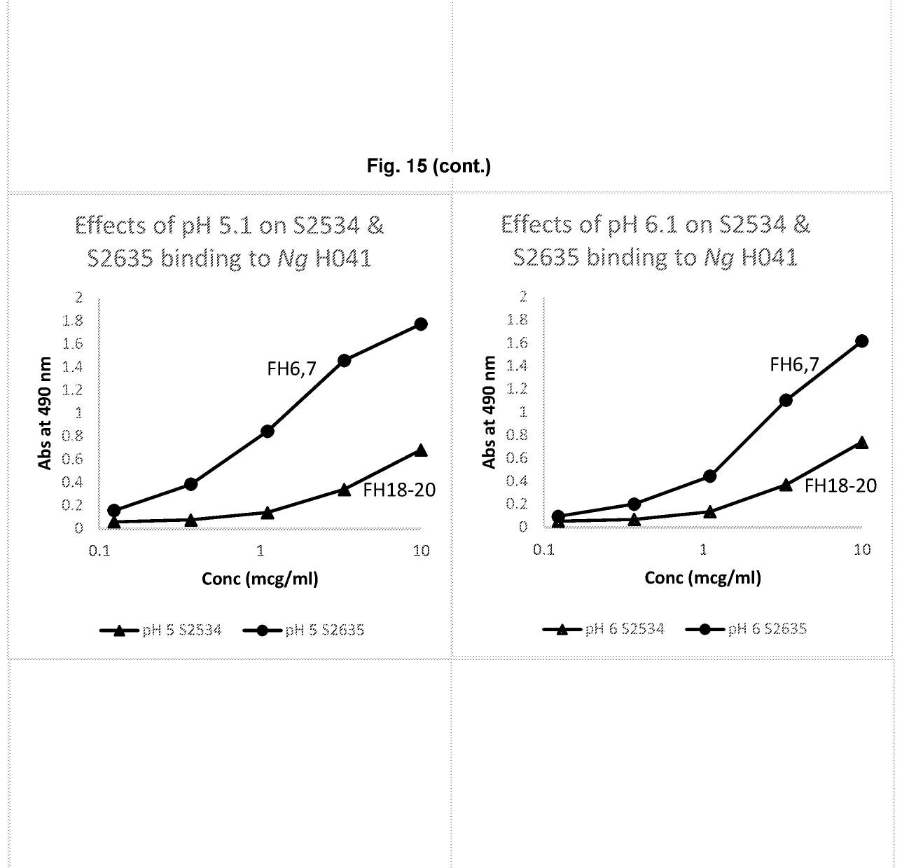

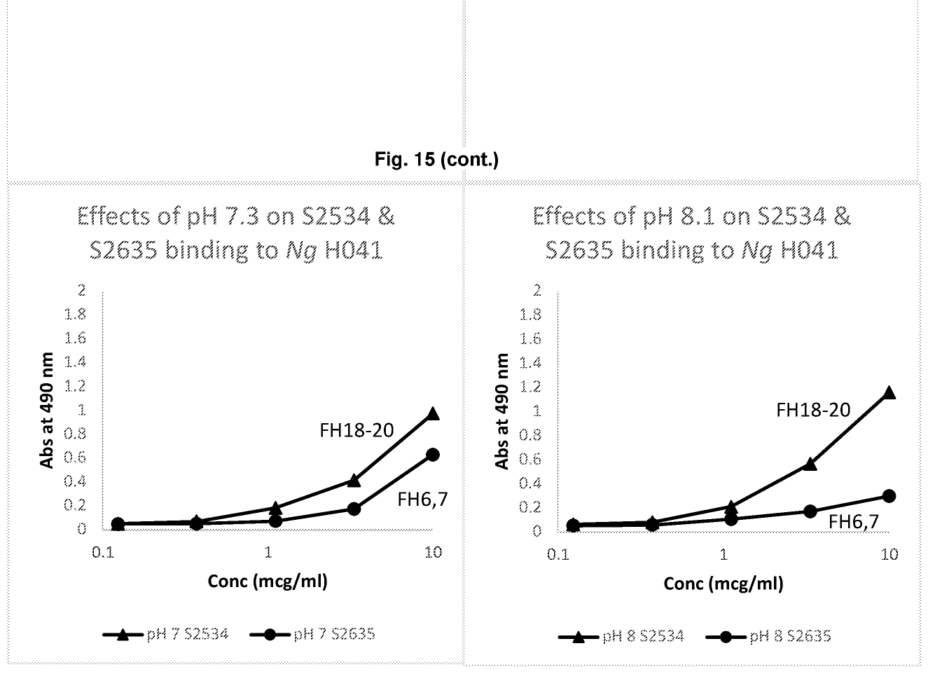

Results: As shown by the results depicted in the plots of FIG. 15, at pH conditions below neutral pH, FH 6,7/Fc bound to Ng H401 more strongly than FH*/Fc. At neutral pH and above, however, FH*/Fc bound to Ng H401 more strongly than FH 6,7/Fc. Thus, in non-pregnant and early pregnant women administration of FH 6,7/Fc for Ng in the lower and upper vagina and lower cervical canal is preferred and administration of FH*/Fc for Ng in the upper cervical, canal lower uterine cavity and upper uterine cavity is preferred. For treatment of the female genital-reproductive tract (vagina, cervical canal and uterine cavity), administration of a mixture comprising FH*/Fc and FH 6,7/Fc is preferred.

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the invention are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

### REFERENCES

1. Ben Nasr, A., and G. R. Klimpel. 2008. Subversion of complement activation at the bacterial surface promotes serum resistance and opsonophagocytosis of Francisella tularensis. J Leukoc Biol 84:77-85.
2. Blaum, B. S., J. P. Hannan, A. P. Herbert, D. Kavanagh, D. Uhrin, and T. Stehle. 2015. Structural basis for sialic acid-mediated self-recognition by complement factor H. Nat Chem Biol 11:77-82.
3. Camara, J., J. Serra, J. Ayats, T. Bastida, D. Carnicer-Pont, A. Andreu and C. Ardanuy (2012). “Molecular characterization of two high-level ceftriaxone-resistant Neisseria gonorrhoeae isolates detected in Catalonia, Spain.” J Antimicrob Chemother 67(8): 1858-1860.
4. Chakraborti, S., L. A. Lewis, A. D. Cox, F. St Michael, J. Li, P. A. Rice and S. Ram (2016). “Phase-Variable Heptose I Glycan Extensions Modulate Efficacy of 2C7 Vaccine Antibody Directed against Neisseria gonorrhoeae Lipooligosaccharide.” J Immunol 196(11): 4576-4586.
5. Dawson, P. E., T. W. Muir, I. Clark-Lewis and S. B. Kent (1994). “Synthesis of proteins by native chemical ligation.” Science 266(5186): 776-779.
6. de Cordoba, S. R., A. Tortajada, C. L. Harris, and B. P. Morgan. 2012. Complement dysregulation and disease: from genes and proteins to diagnostics and drugs. Immunobiology 217:1034-1046.
7. Densen, P., L. A. MacKeen and R. A. Clark (1982). “Dissemination of gonococcal infection is associated with delayed stimulation of complement-dependent neutrophil chemotaxis in vitro.” Infect Immun 38: 563-572
8. Ermert, D., J. Shaughnessy, T. Joeris, J. Kaplan, C. J. Pang, E. A. Kurt-Jones, P. A. Rice, S. Ram and A. M. Blom (2015). “Virulence of Group A Streptococci Is Enhanced by Human Complement Inhibitors.” PLoS pathogens 11(7): e1005043.
9. Ferreira, V. P., A. P. Herbert, C. Cortes, K. A. McKee, B. S. Blaum, S. T. Esswein, D. Uhrin, P. N. Barlow, M. K. Pangburn, and D. Kavanagh. 2009. The binding of factor H to a complex of physiological polyanions and C3b on cells is impaired in atypical hemolytic uremic syndrome. J Immunol 182:7009-7018.
10. Fischer, R., Y. C. Liao and J. Drossard (1999). “Affinity-purification of a TMV-specific recombinant full-size antibody from a transgenic tobacco suspension culture.” J Immunol Methods 226(1-2): 1-10.
11. Gentle, I. E., D. P. De Souza and M. Baca (2004). “Direct production of proteins with N-terminal cysteine for site-specific conjugation.” Bioconjug Chem 15(3): 658-663.
12. Giuntini, S., D. C. Reason, and D. M. Granoff. 2012. Combined roles of human IgG subclass, alternative complement pathway activation, and epitope density in the bactericidal activity of antibodies to meningococcal factor h binding protein. Infect Immun 80:187-194.
13. Giuntini, S., D. M. Granoff, P. T. Beernink, O. Ihle, D. Bratlie, and T. E. Michaelsen. 2016. Human IgG1, IgG3, and IgG3 Hinge-Truncated Mutants Show Different Protection Capabilities against Meningococci Depending on the Target Antigen and Epitope Specificity. Clin Vaccine Immunol 23:698-706.
14. Gulati, S., P. A. Rice and S. Ram (2019). “Complement-Dependent Serum Bactericidal Assays for Neisseria gonorrhoeae.” Methods Mol Biol 1997: 267-280.
15. Hart, T., N. T. T. Nguyen, N. A. Nowak, F. Zhang, R. J. Linhardt, M. Diuk-Wasser, S. Ram, P. Kraiczy, and Y. P. Lin. 2018. Polymorphic factor H-binding activity of CspA protects Lyme borreliae from the host complement in feeding ticks to facilitate tick-to-host transmission. PLoS Pathog 14:e1007106.
16. Hellwage, J., T. S. Jokiranta, M. A. Friese, T. U. Wolk, E. Kampen, P. F. Zipfel, and S. Meri. 2002. Complement C3b/C3d and cell surface polyanions are recognized by overlapping binding sites on the most carboxyl-terminal domain of complement factor H. J Immunol 169:6935-6944.
17. Hezareh, M., A. J. Hessell, R. C. Jensen, J. G. van de Winkel and P. W. Parren (2001). “Effector function activities of a panel of mutants of a broadly neutralizing antibody against human immunodeficiency virus type 1.” J Virol 75(24): 12161-12168.
18. Hitchcock, P. J., S. F. Hayes, L. W. Mayer, W. M. Shafer and S. L. Tessier (1985). “Analyses of gonococcal H.8 antigen: surface location, inter- and intrastrain electrophoretic heterogeneity, and unusual two-dimensional electrophoretic characteristics.” J. Exp. Med. 162(6): 2017-2034.
19. Idusogie, E. E., L. G. Presta, H. Gazzano-Santoro, K. Totpal, P. Y. Wong, M. Ultsch, Y. G. Meng, and M. G. Mulkerrin. 2000. Mapping of the C1q binding site on rituxan, a chimeric antibody with a human IgG1 Fc. J Immunol 164:4178-4184.
20. Idusogie, E. E., P. Y. Wong, L. G. Presta, H. Gazzano-Santoro, K. Totpal, M. Ultsch, and M. G. Mulkerrin. 2001. Engineered antibodies with increased activity to recruit complement. J Immunol 166:2571-2575.
21. Jokiranta, T. S., Z. Z. Cheng, H. Seeberger, M. Jozsi, S. Heinen, M. Noris, G. Remuzzi, R. Ormsby, D. L. Gordon, S. Meri, J. Hellwage, and P. F. Zipfel. 2005. Binding of complement factor H to endothelial cells is mediated by the carboxy-terminal glycosaminoglycan binding site. Am J Pathol 167:1173-1181.
22. Jokiranta, T. S., V. P. Jaakola, M. J. Lehtinen, M. Parepalo, S. Meri and A. Goldman (2006). “Structure of complement factor H carboxyl-terminus reveals molecular basis of atypical haemolytic uremic syndrome.” EMBO J 25(8): 1784-1794
23. Kajander, T., M. J. Lehtinen, S. Hyvarinen, A. Bhattacharjee, E. Leung, D. E. Isenman, S. Meri, A. Goldman, and T. S. Jokiranta. 2011. Dual interaction of factor H with C3d and glycosaminoglycans in host-nonhost discrimination by complement. Proc Natl Acad Sci USA 108:2897-2902.
24. Kolodziejczyk, R., K. M. Mikula, T. Kotila, V. L. G. Postis, T. S. Jokiranta, A. Goldman, and T. Meri. 2017. Crystal structure of a tripartite complex between C3dg, C-terminal domains of factor H and OspE of Borrelia burgdorferi. PLoS One 12:e0188127.
25. Konar, M. and D. M. Granoff (2017). “Eculizumab treatment and impaired opsonophagocytic killing of meningococci by whole blood from immunized adults.” Blood 130(7): 891-899.
26. Koncz, C. and J. Schell (1986). “The promoter of TL-DNA gene 5 controls the tissue-specific expression of chimaeric genes carried by a novel type of Agrobacterium binary vector.” Molecular and General Genetics 204( ) 383-396.
27. Kraiczy, P., and B. Stevenson. 2013. Complement regulator-acquiring surface proteins of Borrelia burgdorferi: Structure, function and regulation of gene expression. Ticks Tick Borne Dis 4:26-34.
28. Lewis, L. A., S. Ram, A. Prasad, S. Gulati, S. Getzlaff, A. M. Blom, U. Vogel and P. A.

Rice (2008). “Defining targets for complement components C4b and C3b on the pathogenic neisseriae.” Infect Immun 76(1): 339-350.
29. Liang, F. T., A. C. Steere, A. R. Marques, B. J. Johnson, J. N. Miller, and M. T. Philipp. 1999. Sensitive and specific serodiagnosis of Lyme disease by enzyme-linked immunosorbent assay with a peptide based on an immunodominant conserved region of Borrelia burgdorferi vlsE. J Clin Microbiol 37:3990-3996.
30. Lin, Y. P., A. M. Frye, T. A. Nowak, and P. Kraiczy. 2020. New Insights Into CRASP-Mediated Complement Evasion in the Lyme Disease Enzootic Cycle. Front Cell Infect Microbiol 10:1.
31. Lykke M R, Becher N, Haahr T, Boedtkjer E, Jensen J S, Uldbjerg N. Vaginal, Cervical and Uterine pH in Women with Normal and Abnormal Vaginal Microbiota. Pathogens. 2021 Jan. 20; 10(2):90. doi: 10.3390/pathogens10020090. PMID: 33498288; PMCID: PMC7909242.
32. Maclean, J., M. Koekemoer, A. J. Olivier, D. Stewart, Hitzeroth, II, T. Rademacher, R. Fischer, A. L. Williamson and E. P. Rybicki (2007). “Optimization of human papillomavirus type 16 (HPV-16) L1 expression in plants: comparison of the suitability of different HPV-16 L1 gene variants and different cell-compartment localization.” J Gen Virol 88(Pt 5): 1460-1469.
33. Manuelian, T., J. Hellwage, S. Meri, J. Caprioli, M. Noris, S. Heinen, M. Jozsi, H. P. Neumann, G. Remuzzi, and P. F. Zipfel. 2003. Mutations in factor H reduce binding affinity to C3b and heparin and surface attachment to endothelial cells in hemolytic uremic syndrome. J Clin Invest 111:1181-1190.
34. Martensson, L., E. van Loghem, H. Matsumoto, and J. Nielsen. 1966. Gm(s) and Gm(t): genetic determinants of human gamma-globulin. Vox Sang 11:393-418.
35. Natsume, A., M. In, H. Takamura, T. Nakagawa, Y. Shimizu, K. Kitajima, M. Wakitani, S. Ohta, M. Satoh, K. shitara, and R. Niwa. 2008. Engineered antibodies of IgG1/IgG3 mixed isotype with enhanced cytotoxic activities. Cancer Res 68:3863-3872.
36. Ohnishi, M., D. Golparian, K. Shimuta, T. Saika, S. Hoshina, K. Iwasaku, S. Nakayama, J. Kitawaki and M. Unemo (2011). “Is Neisseria gonorrhoeae initiating a future era of untreatable gonorrhea?: detailed characterization of the first strain with high-level resistance to ceftriaxone.” Antimicrob Agents Chemother 55(7): 3538-3545.
37. Riley, S. P., J. L. Patterson, and J. J. Martinez. 2012. The rickettsial OmpB beta-peptide of Rickettsia conorii is sufficient to facilitate factor H-mediated serum resistance. Infect Immun 80:2735-2743.
38. Rottgerding, F., A. Wagemakers, J. Koetsveld, V. Fingerle, M. Kirschfink, J. W. Hovius, P. F. Zipfel, R. Wallich, and P. Kraiczy. 2017. Immune evasion of Borrelia miyamotoi: CbiA, a novel outer surface protein exhibiting complement binding and inactivating properties. Scientific reports 7:303.
39. Sarantis, H. and S. D. Gray-Owen (2007). “The specific innate immune receptor CEACAM3 triggers neutrophil bactericidal activities via a Syk kinase-dependent pathway.” Cell Microbiol 9(9): 2167-2180.
40. Shaughnessy, J., S. Gulati, S. Agarwal, M. Unemo, M. Ohnishi, X. H. Su, B. G. Monks, A. Visintin, G. Madico, L. A. Lewis, D. T. Golenbock, G. W. Reed, P. A. Rice and S. Ram (2016). “A Novel Factor H-Fc Chimeric Immunotherapeutic Molecule against Neisseria gonorrhoeae.” Journal of immunology 196(4): 1732-1740.
41. Shaughnessy, J., L. A. Lewis, B. Zheng, C. Carr, I. Bass, S. Gulati, R. B. DeOliveira, S. Gose, G. W. Reed, M. Botto, P. A. Rice and S. Ram (2018). “Human Factor H Domains 6 and 7 Fused to IgG1 Fc Are Immunotherapeutic against Neisseria gonorrhoeae.” J Immunol 201(9): 2700-2709.
42. Shen, W. J. and B. G. Forde (1989). “Efficient transformation of Agrobacterium spp. by high voltage electroporation.” Nucleic Acids Res 17(20): 8385.
43. Schmidt, C. Q., A. P. Herbert, D. Kavanagh, C. Gandy, C. J. Fenton, B. S. Blaum, M. Lyon, D. Uhrin, and P. N. Barlow. 2008. A new map of glycosaminoglycan and C3b binding sites on factor H. J Immunol 181:2610-2619.
44. Schneider, H., J. M. Griffiss, J. W. Boslego, P. J. Hitchcock, K. M. Zahos and M. A. Apicella (1991). “Expression of paragloboside-like lipooligosaccharides may be a necessary component of gonococcal pathogenesis in men.” J Exp Med 174: 1601-1605.
45. Shafer, W. M., K. Joiner, L. F. Guymon, M. S. Cohen and P. F. Sparling (1984). “Serum sensitivity of Neisseria gonorrhoeae: the role of lipopolysaccharide.” J Infect Dis 149(2): 175-183.
46. Skare, J. T., and B. L. Garcia. 2020. Complement Evasion by Lyme Disease Spirochetes. Trends Microbiol 28:889-899.
47. Stapleton, N. M., J. T. Andersen, A. M. Stemerding, S. P. Bjarnarson, R. C. Verheul, J. Gerritsen, Y. Zhao, M. Kleijer, I. Sandlie, M. de Haas, I. Jonsdottir, C. E. van der Schoot, and G. Vidarsson. 2011. Competition for FcRn-mediated transport gives rise to short half-life of human IgG3 and offers therapeutic potential. Nat Commun 2:599.
48. Steinberg, A. G. 1969. Globulin polymorphisms in man. Annu. Rev. Genet. 3: 25-52.
49. Strasser, R., J. Stadlmann, M. Schahs, G. Stiegler, H. Quendler, L. Mach, J. Glossl, K. Weterings, M. Pabst and H. Steinkellner (2008). “Generation of glyco-engineered Nicotiana benthamiana for the production of monoclonal antibodies with a homogeneous human-like N-glycan structure.” Plant biotechnology journal 6(4): 392-402.
50. Ueda, Y., T. Miwa, D. Ito, H. Kim, S. Sato, D. Gullipalli, L. Zhou, M. Golla, D. Song, J. L. Dunaief, M. B. Palmer and W. C. Song (2019). “Differential contribution of C5aR and C5b-9 pathways to renal thrombic microangiopathy and macrovascular thrombosis in mice carrying an atypical hemolytic syndrome-related factor H mutation.” Kidney Int. 07; 96(1):67-79.
51. Unemo, M., D. Golparian, L. Sanchez-Buso, Y. Grad, S. Jacobsson, M. Ohnishi, M. M. Lahra, A. Limnios, A. E. Sikora, T. Wi and S. R. Harris (2016). “The novel 2016 WHO Neisseria gonorrhoeae reference strains for global quality assurance of laboratory investigations: phenotypic, genetic and reference genome characterization.” J Antimicrob Chemother 71(11): 3096-3108.
52. Voinnet, O., S. Rivas, P. Mestre and D. Baulcombe (2003). “An enhanced transient expression system in plants based on suppression of gene silencing by the p19 protein of tomato bushy stunt virus.” Plant J 33(5): 949-956.
53. Voss, A., M. Niersbach, R. Hain, H. J. Hirsch, Y. C. Liao, F. Kreuzaler and R. Fischer (1995). Reduced virus infectivity in N. tabacum secreting a TMV-specific full-size antibody. Molecular breeding: new strategies in plant improvement. Dordrecht; Boston, Kluwer Academic Publishers. 1: 39-50.
54. Vaquero, C., M. Sack, J. Chandler, J. Drossard, F. Schuster, M. Monecke, S. Schillberg and R. Fischer (1999). “Transient expression of a tumor-specific single-chain fragment and a chimeric antibody in tobacco leaves.” Proceedings of the National Acadamy of Sciences USA 96: 11128-11133.
55. Wong, S. M., J. Shaughnessy, S. Ram, and B. J. Akerley. 2016. Defining the Binding Region in Factor H to Develop a Therapeutic Factor H-Fc Fusion Protein against Non-Typeable Haemophilus influenzae. Front Cell Infect Microbiol 6:40.
56. Wycoff, K. L., A. Belle, D. Deppe, L. Schaefer, J. M. Maclean, S. Haase, A. K. Trilling, S. Liu, S. H. Leppla, I. N. Geren, J. Pawlik and J. W. Peterson (2011). “Recombinant anthrax toxin receptor-Fc fusion proteins produced in plants protect rabbits against inhalational anthrax.” Antimicrob Agents Chemother 55(1): 132-139.
57. Wycoff, K., J. Maclean, A. Belle, L. Yu, Y. Tran, C. Roy and F. Hayden (2015). “Anti-infective immunoadhesins from plants.” Plant Biotechnol J 13(8): 1078-1093.

1. A fusion protein comprising an Fc and at least one Factor H (FH) short consensus repeat (SCR) domain capable of binding to a pathogen, wherein the Fc and FH SCR domains are fused by a linker consisting of glycine and serine residues; optionally, wherein the at least one FH SCR domain is selected from the group consisting of SCR 20, SCR 19-20, SCR 18-20, and SCR 6-7.

2. The fusion protein of claim 1, wherein the at least one FH SCR domain is domain 19 and has a point mutation at position 1119 which abrogates binding to host cells.

3. The fusion protein of claim 1, wherein:
(a) the number of glycine residues exceeds the number of serine residues in the linker;
(b) wherein the ratio of glycine residues to serine residues in the linker is 4 to 1;
(c) the linker is selected from the group consisting of GGGGS, (GGGGS)₂ and (GGGGS)₃; and/or
(d) the linker comprises an amino acid sequence selected from SEQ ID NO: 38-43.

4. (canceled)

5. (canceled)

6. (canceled)

7. The fusion protein of claim 1, wherein:
(a) the at least one FH SCR is at the N-terminus and the Fc is at the C-terminus of the fusion protein; or
(b) the at least one FH SCR is at the C-terminus and said the Fc is at the N-terminus of the fusion protein.

8. (canceled)

9. (canceled)

10. The fusion protein of claim 1, wherein:
(a) said Fc comprises Fc of human IgG1 and further comprises the hinge region of IgG1; optionally, wherein the hinge region comprises an amino acid sequence selected from SEQ ID NO: 3, and 4; or
(b) said Fc comprises Fc of human IgG1 and further comprises the hinge region of IgG3; optionally, wherein the hinge region comprises an amino acid sequence selected from SEQ ID NO: 5, and 23.

11. (canceled)

12. The fusion protein of claim 2 further comprising additional N-terminal amino acids attached to FH*, wherein the additional N-terminal amino acids are selected from the group consisting of: TS (threonine, and serine); DTS (aspartic acid, threonine, and serine); and RDTS (arginine, aspartic acid, threonine, and serine).

13. The fusion protein of claim 12, wherein:
(a) the amino acid sequence of the fusion protein has the linear structure: N-terminus-[additional N-terminal amino acids]-FH*-linker-Fc-C-terminus: or
(b) the amino acid sequence of the fusion protein has the linear structure: N-terminus-Fc-linker-[additional N-terminal amino acids]-FH*-C-terminus.

14. (canceled)

15. The fusion protein of claim 13, wherein the fusion protein has the linear structure selected from:
(i) N-terminus-Fc-linker-TS-FH*-C-terminus;
(ii) N-terminus-Fc-linker-DTS-FH*-C-terminus; and
(iii) N-terminus-Fc-linker-RDTS-FH*-C-terminus.

16. (canceled)

17. (canceled)

18. (canceled)

19. The fusion protein of claim 1, wherein the at least one FH SCR domain is SCR 20, and the SCR 20 domain comprises amino acid modifications selected from the group consisting of: R1203E, R1206E, and R1210S or R1203L/R1206N/R1210S.

20. The fusion protein of claim 1, wherein: (a) the Fc is IgG3 Fc and comprises amino acid modifications thereof selected from the group consisting of: M252Y/S254T/T256E or M428L/N434S; wherein the Fc comprises the amino acid sequence of IgHg3*17.

21. (canceled)

22. A polynucleotide encoding a fusion protein of claim 1.

23. An expression vector comprising a polynucleotide of claim 22, wherein:
(a) the vector is suitable for expressing the polynucleotide in a mammalian host cell or a mammalian tissue; optionally, wherein the mammalian cell or tissue comprises a CHO cell; or
(b) the vector is suitable for expressing the polynucleotide in a plant cell or plant tissue;
optionally, wherein plant cell or tissue is from N. benthamiana.

24. (canceled)

25. A method for improving the microbiocidal efficacy of FH 6-7/Fc and FH*/Fc fusions by providing the fusion protein of claim 1 in the presence of complement.

26. A method for improving the opsinophagocytotic efficacy of FH 6-7/Fc and FH*/Fc fusions by providing the fusion protein of claim 1 in the presence of PMN and complement.

27. A method for reducing the duration and/or burden of colonization of a microbe in a mammalian host, the method comprising providing to the mammalian host a fusion protein of claim 1 in an amount effective to reduce the duration and/or burden of colonization.

28. A method for reducing a population of pathogenic microbes in an organism, the method comprising treating the organism with an effective amount of a fusion protein of claim 1.

29. A method for preventing and/or treating a microbe infection in a subject, the method comprising administering to the subject an effective amount of a fusion protein of claim 1.

30. The method of claim 29, wherein said microbes are selected from the group consisting of Neisseria gonorrhoeae (Ng), N. meningitidis, group A streptococci, methicillin resistant Staphylococcus aureus non-typeable Haemophilus influenzae, Borrelia burgdorferi sensu lato (collectively referred to as the Lyme borreliae), B. burgdorferi sensu stricto (Bb) and B. afzelii (Ba), B. garinii (Bg), B. bavariensis (Bbav), Borrelia miyamotoi (Bm), Rickettsia sp., and Francisella tularensis.
